SEQUENCE LISTING (23 sequences)

SEQ ID NO: 1 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 2 - DNA, 3659 bp, Homo sapiens
acaagcgagctggtggttgaagctggttaaagaacagcctaggtattccagaagtgtttgaggatcccttccatgaaggaagagaggaaagtttttaagtaaacctcccactcccatgtgtcttcagctttcttttgcaaaggagaaaatccttgaagtttggtaaagaccgagttagtctatctctctttgcctatctcgagttgggctggggagaggaggagataggttcttttgtctttttctgtcttctcccttccccacttccttccctccagtccccactcactcacatgcacacactaaccttggagccgatgggattgagtgactggcacttgggaccacagagaaatgtcagagtgtttggttacagactcaaggaaacctctcattttagagtgctcatttggttttgagcaaaattttggactgtgaagcaaggcattggtgaagacaaaatggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgctgaagcatggagtgttgatgctttcgtctccagccccttctcagaatctacccaaaggatactgtaacactttacaatgttcatcccatgatgtagtgcctctttcatccactagtgcaaatcatagctgggggttgggggtggtgggggtcggggcctgggggactgggagccgcagcagctccccctcccccactgccataaaacattaagaaaatcatattgcttcttctcctatgtgtaaggtgaaccatgtcagcaaaaagcaaaatcattttatatgtcaaagcaggggagtatgcaaaagttctgacttgactttagtctgcaaaatgaggaatgtatatgttttgtgggaacagatgtttcttttgtatgtaaatgtgcattcttttaaaagacaagacttcagtatgttgtcaaagagagggctttaatttttttaaccaaaggtgaaggaatatatggcagagttgtaaatatataaatatatatatatataaaataaatatatataaacctaaaaaagatatattaaaaatataaaactgcgttaaaggctcgattttgtatctgcaggcagacacggatctgagaatctttattgagaaagagcacttaagagaatattttaagtattgcatctgtataagtaagaaaatattttgtctaaaatgcctcagtgtatttgtatttttttgcaagtgaaggtttacaatttacaaagtgtgtattaaaaaaaacaaaaagaacaaaaaaatctgcagaaggaaaaatgtgtaattttgttctagttttcagtttgtatatacccgtacaacgtgtcctcacggtgccttttttcacggaagttttcaatgatgggcgagcgtgcaccatccctttttgaagtgtaggcagacacagggacttgaagttgttactaactaaactctctttgggaatgtttgtctcatcccattctgcgtcatgcttgtgttataactactccggagacagggtttggctgtgtctaaactgcattaccgcgttgtaaaatatagctgtacaaatataagaataaaatgttgaaaagtcaaa

SEQ ID NO: 3 - protein, 6 aa, Artificial Sequence
  Description of Artificial Sequence Synthetic 6xHis tag
HHHHHH

SEQ ID NO: 4 - DNA, 45 bp, Artificial Sequence
  Description of Artificial Sequence Synthetic oligonucleotide
tccggagtcgagacatcttgaaatgcaaccttagtgtgatcaatc

SEQ ID NO: 5 - DNA, 45 bp, Artificial Sequence
  Description of Artificial Sequence Synthetic oligonucleotide
ctatagcatctttacagaccagttgggcaacatcatggctgtgat

SEQ ID NO: 6 - protein, 17 aa, Artificial Sequence
  Description of Artificial Sequence Synthetic peptide
RSEIISTAPSSWVVPGP

SEQ ID NO: 7 - protein, 68 aa, Artificial Sequence
  Description of Artificial Sequence Synthetic polypeptide
TDVVIVVSREQFRAHKTVLMRCSGLFYSIFTDQLKCNLSVINLDPEINPEGFCILLDFMYTSRLNLRE

SEQ ID NO: 8 - protein, 11 aa, Unknown
  Description of Unknown PHYL sequence
LRPVAMVRPTV

SEQ ID NO: 9 - protein, 11 aa, Unknown
  Description of Unknown OBF-1 sequence
PAPTAVVLPHQ

SEQ ID NO: 10 - protein, 11 aa, Unknown
  Description of Unknown SIP sequence
EKPAAVVAPIT

SEQ ID NO: 11 - protein, 11 aa, Unknown
  Description of Unknown DCC sequence
TIPTACVRPTH

SEQ ID NO: 12 - protein, 11 aa, Unknown
  Description of Unknown TIEG-1 sequence
NIPCAAVSPNR

SEQ ID NO: 13 - protein, 11 aa, Unknown
  Description of Unknown NUMB sequence
TKPVTVVAPQS

SEQ ID NO: 14 - protein, 11 aa, Unknown
  Description of Unknown EF1-D sequence
VSPMRQVEPPA

SEQ ID NO: 15 - protein, 11 aa, Unknown
  Description of Unknown VAV sequence
WFPCNRVRPYV

SEQ ID NO: 16 - protein, 11 aa, Unknown
  Description of Unknown KID sequence
PLKKAVVMPLQ

SEQ ID NO: 17 - protein, 11 aa, Unknown
  Description of Unknown NCOR sequence
QRISAAVLPLV

SEQ ID NO: 18 - protein, 11 aa, Unknown
  Description of Unknown FIR sequence
PGVITGVTPAR

SEQ ID NO: 19 - protein, 11 aa, Unknown
  Description of Unknown CDC42 sequence
AAPTATVRPMP

SEQ ID NO: 20 - protein, 11 aa, Unknown
  Description of Unknown T-STAR sequence
ARPVGVVVPRG

SEQ ID NO: 21 - protein, 11 aa, Homo sapiens
SRPTLEVSPNV

SEQ ID NO: 22 - protein, 11 aa, Artificial Sequence
  Description of Artificial Sequence Synthetic peptide
SRPTLEGSANV

SEQ ID NO: 23 - protein, 10 aa, Artificial Sequence
  Description of Artificial Sequence Synthetic peptide
GGGGSGGGGS

(19) United States
(12) Patent Application Publication
(10) Pub. No.: US 2022/0281931 A1
(43) Pub. Date: Sep. 8, 2022

(54) CHEMICALLY INDUCIBLE POLYPEPTIDE POLYMERIZATION

(71) Applicants: Dana-Farber Cancer Institute, Inc., Boston, MA (US); Deutsches Krebsforschungszentrum Stiftung des öffentlichen Rechts, Heidelberg (DE)

(72) Inventors: Benjamin EBERT, Boston, MA (US); Mikolaj SLABICKI, Boston, MA (US); Eric S. FISCHER, Boston, MA (US); Hojong YOON, Boston, MA (US); Jonas KOEPPEL, Heidelberg (DE)

(73) Assignees: Dana-Farber Cancer Institute, Inc., Boston, MA (US); Deutsches Krebsforschungszentrum Stiftung des öffentlichen Rechts, Heidelberg (DE)

(21) Appl. No.: 17/750,061

(22) Filed: May 20, 2022

Related U.S. Application Data
(63) Continuation of application No. PCT/US2020/061542, filed on Nov. 20, 2020.
(60) Provisional application No. 63/074,279, filed on Sep. 3, 2020.
(60) Provisional application No. 62/938,736, filed on Nov. 21, 2019.

(51) Int. Cl. C07K 14/47; G01N 33/68

(52) U.S. Cl. CPC C07K 14/4702; G01N 33/68; C07K 2319/95

(57) ABSTRACT

The invention features methods for characterizing a cancer as sensitive or resistant to Bcl6 therapies, as well as compositions and methods for inducing the degradation or polymerization of a polypeptide of interest.

### CROSS-REFERENCE TO RELATED APPLICATIONS

This application is a continuation application, pursuant to 35 U.S.C. § 111(a) of PCT International Application No. PCT/US2020/061542, filed Nov. 20, 2020 designating the United States and published in English, which claims the benefit of and priority to U.S. Provisional Application No. 63/074,279, filed Sep. 3, 2020 and U.S. Provisional Application No. 62/938,736, filed Nov. 21, 2019, the entire contents of each of which are incorporated herein by reference in their entirety.

### STATEMENT OF RIGHTS TO INVENTIONS MADE UNDER FEDERALLY SPONSORED RESEARCH

This invention was made with government support under Grant Nos. HG082945, CA108631, CA206963, CA214608, and CA218278 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jan. 4, 2021, is named 167741_022502PCT_SL.txt and is 16,380 bytes in size.

### BACKGROUND OF THE INVENTION

Small molecule-induced protein degradation is a powerful therapeutic strategy, as demonstrated by the clinical efficacy of thalidomide analogs for the treatment of hematologic malignancies. Thalidomide analogs, including lenalidomide and pomalidomide, modulate the activity of the CUL4-RBX1-DDB1-CRBN (CRL4^{CRBN}) E3 ubiquitin ligase to recruit and ubiquitinate neo-substrates including IKZF1, IKZF3, and CK1α, which leads to their proteasomal degradation. Other small molecules that induce protein degradation include aryl sulfonamides, which promote the destruction of RBM39 in a CUL4-RBX1-DDB1-DCAF15 (CRL4^{DCAF15})-dependent manner, and hetero-bifunctional degraders (also known as PROTACs) that have been developed for a wide range of targets including kinases, nuclear receptors and epigenetic enzymes. These small molecule degraders engage both the E3 ligase and the target protein substrate, promoting formation of a substrate-drug-ligase ternary complex. While degraders can show remarkable efficacy and sustained target depletion, some proteins have proven recalcitrant to this approach. One such example is the B cell lymphoma 6 (BCL6) protein, for which hetero-bifunctional degraders have shown insufficient target modulation to induce growth inhibition.

BCL6 is a promising drug target for non-Hodgkin lymphomas such as diffuse large B cell lymphoma (DLBCL) and follicular lymphoma. Pathologically increased BCL6 expression, as a result of somatic BCL6 translocation, exonic mutation, promoter mutation, or mutations in regulatory pathways, may be a driver of B cell malignancies. In genetically engineered mice, overexpression of BCL6 is sufficient to drive lymphoma development. BCL6 acts as a master transcriptional repressor enabling rapid expansion of germinal center (GC) B cells and tolerance to genomic instability caused by hypermutation of the immunoglobulin genes and class switch recombination. BCL6 represses a broad range of genes involved in the DNA damage response, cell cycle checkpoints, and differentiation. Knock-out of BCL6 in lymphoma cells results in tumor stasis. Several peptide and small molecule inhibitors targeting BCL6 have shown efficacy in vivo, but only at high concentrations, which has limited their translation into clinical therapeutic agents.

Screens for novel BCL6 inhibitors led to the identification of small molecules that induce BCL6 degradation, including BI-3802. These molecules bind the Broad complex/Tramtrack/Bric-a-brac (BTB) domain, which mediates BCL6 homodimerization and its interactions with co-repressor proteins. BI-3802 induces rapid ubiquitination and degradation of BCL6, resulting in profound de-repression of BCL6 target genes and anti-proliferative effects in DLBCL cell lines, comparable to a genetic knock-out, and superior to non-degrading BCL6 inhibitors such as BI-3812 or heterobifunctional BCL6 degraders.

There remains a need to develop better methods for evaluating whether a cancer may be sensitive to or resistant to treatment with a small molecule drug.

### SUMMARY OF THE INVENTION

As described below, the present invention features methods for characterizing a cancer as sensitive or resistant to BCL6 therapies, as well as compositions and methods for inducing the degradation or polymerization of a polypeptide of interest.

In one aspect, the invention features a recombinant polypeptide including a fragment of a BCL6 polypeptide, where the fragment includes a Broad complex/Tamtrack/Brick-a-brack (BTB) domain of the BCL6 polypeptide.

In other aspects, the invention features a fusion polypeptide including a BTB domain linked to a heterologous amino acid sequence.

In various embodiments, the polypeptide further includes a degron. In some embodiments, the polypeptide includes an amino acid linker at an N-terminus or a C-terminus of the BTB domain. In some embodiments, the BTB domain is operably linked to the degron via a linker. In some embodiments, the degron includes at least about 20 amino acid residues of a BCL6 polypeptide. In some embodiments, the degron includes amino acid residues 241-260 of a BCL6 polypeptide. In some embodiments, the degron includes amino acid residues 249-251 of a BCL6 polypeptide. In various embodiments, the degron includes a V×P binding motif.

In some embodiments, the polypeptide includes amino acids 1-275 of the BCL6 polypeptide. In various embodiments, the polypeptide includes amino acids 1-129 of the BCL6 polypeptide.

In some embodiments, the polypeptide includes an alteration at amino acid R28, E41, C84, G55, or Y58. In some embodiments, the polypeptide does not include an alteration at amino acid R28, E41, C84, G55, or Y58. In some embodiments, the polypeptide includes an alteration selected from the group consisting of R28A, E41A, C84A, G55A, and Y58A.

In various embodiments, the polypeptide is polymerizable. In some embodiments, polymerization of the polypeptide enhances degradation of the polypeptide. In various embodiments, the polypeptide is reversibly polymerizable.

In some embodiments, polymerization of the polypeptide is induced by a compound binding to the BTB domain. In some embodiments, the BTB includes a hydrophobic residue that mediates polymerization of the polypeptide upon binding of the compound. In various embodiments, the compound is a quinolinone. In various embodiments, the compound is a benzimidazolone. In some embodiments, the compound is selected from the group consisting of BI-3802 (2-((6-((5-chloro-2-((3S,5R)-3,5-dimethylpiperidin-1-yl)pyrimidin-4-yl)amino)-1-methyl-2-oxo-1,2-dihydroquinolin-3-yl)oxy)-N-methylacetamide); 5-((5-Chloro-2-(3-methylpiperidin-1-yl)pyrimidin-4-yl)-amino)-3-(3-hydroxy-3-methylbutyl)-1-methyl-1,3-dihydro-2H-benzo[d]imidazol-2-one; 5-((5-Chloro-2-((3S,5R)-3,5-dimethylpiperidin-1-yl)-pyrimidin-4-yl)amino)-3-(3-hydroxy-3-methylbutyl)-1-methyl-1,3-dihydro-2H-benzo[d]imidazol-2-one; 5-((5-Chloro-2-((3S,5R)-3,5-dimethylpiperidin-1-yl)pyridin-4-yl)amino)-3-(3-hydroxy-3-methylbutyl)-1-methyl-1,3-dihydro-2H-benzo[d]imidazol-2-one; 5-((5-Chloro-2-(3-(trifluoromethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(4,4-difluoropiperidin-1-yl)pyrimidin-4-yl)-amino)-3-(3-hydroxy-3-methylbutyl)-1-methyl-1,3-dihydro-2H-benzo[d]imidazol-2-one; 5-((5-Chloro-2-(4,4-difluoro-3-methylpiperidin-1-yl)-pyrimidin-4-yl)amino)-3-(3-hydroxy-3-methylbutyl)-1-methyl-1,3-dihydro-2H-benzo[d]imidazol-2-one; 5-((5-Chloro-2-((3R,5S)-4,4-difluoro-3,5-dimethylpiperidin-1-yl)pyrimidin-4-yl)amino)-3-(3-hydroxy-3-methylbutyl)-1-methyl-1,3-dihydro-2H-benzo-[d]imidazol-2-one, 5-((5-Chloro-2-(3-(hydroxymethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(4,4-difluoro-3-(hydroxymethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(4,4-difluoro-3-(methoxymethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(dimethylamino)pyrimidin-4-yl)amino)-3-(3-hydroxy-3-methylbutyl)-1-methyl-1,3-dihydro-2H-benzo[d]-imidazol-2-one; 5-((5-Chloro-2-morpholinopyrimidin-4-yl)amino)-3-(3-hydroxy-3-methylbutyl)-1-methyl-1,3-dihydro-2H-benzo[d]-imidazol-2-one; 5-((5-Chloro-2-(piperidin-1-yl)pyrimidin-4-yl)amino)-3-(3-hydroxy-3-methylbutyl)-1-methyl-1,3-dihydro-2H-benzo[d]-imidazol-2-one; 5-((5-Chloro-2-((2R,6S)-2,6-dimethylmorpholino)pyridin-4-yl)amino)-3-(3-hydroxy-3-methylbutyl)-1-methyl-1,3-dihydro-2H-benzo[d]imidazol-2-one; 2-Chloro-4-((3-(3-hydroxy-3-methylbutyl)-1-methyl-2-oxo-2,3-dihydro-1H-benzo[d]imidazol-5-yl)amino)nicotinonitrile; 5-((2,3-Dichloropyridin-4-yl)amino)-3-(3-hydroxy-3-methylbutyl)-1-methyl-1,3-dihydro-2H-benzo[d]imidazol-2-one; 5-((3-Chloropyridin-4-yl)amino)-3-(3-hydroxy-3-methylbutyl)-1-methyl-1,3-dihydro-2H-benzo[d]imidazol-2-one; 4-Chloro-6-((3-(3-hydroxy-3-methylbutyl)-1-methyl-2-oxo-2,3-dihydro-1H-benzo[d]imidazol-5-yl)amino)pyrimidine-5-Carbonitrile; 5-((5,6-Dichloropyrimidin-4-yl)amino)-3-(3-hydroxy-3-methylbutyl)-1-methyl-1,3-dihydro-2H-benzo[d]imidazol-2-one; 5-((3,5-Dichloropyridin-4-yl)amino)-3-(3-hydroxy-3-methylbutyl)-1-methyl-1,3-dihydro-2H-benzo[d]imidazol-2-one; 5-((5-Chloro-2-(methylthio)pyrimidin-4-yl)amino)-3-(3-hydroxy-3-methylbutyl)-1-methyl-1,3-dihydro-2H-benzo[d]-imidazol-2-one; 5-((2,5-Dichloropyrimidin-4-yl)amino)-3-(3-hydroxy-3-methylbutyl)-1-methyl-1,3-dihydro-2H-benzo[d]imidazol-2-one; 2-Chloro-4-((cyclopropylmethyl)amino)nicotinonitrile; 3,4,2-Chloro-4-((1,3-dimethyl-2-oxo-2,3-dihydro-1H-benzo[d]-imidazol-5-yl)amino)nicotinonitrile; 2-Chloro-4-((3-(2-hydroxybutyl)-1-methyl-2-oxo-2,3-dihydro-1H-benzo[d]imidazol-5-yl)amino)nicotinonitrile; 2-Chloro-4-((3-(2-cyanobutyl)-1-methyl-2-oxo-2,3-dihydro-1H-benzo[d]imidazol-5-yl)amino)nicotinonitrile; (S)-2-Chloro-4-((3-(2-hydroxybutyl)-1-methyl-2-oxo-2,3-dihydro-1H-benzo[d]imidazol-5-yl)amino)nicotinonitrile; (R)-2-Chloro-4-((3-(2-hydroxybutyl)-1-methyl-2-oxo-2,3-dihydro-1H-benzo[d]imidazol-5-yl)amino)nicotinonitrile; 4-((3-Butyl-1-methyl-2-oxo-2,3-dihydro-1H-benzo[d]-imidazol-5-yl)amino)-2-chloronicotinonitrile; (R)-2-Chloro-4-((3-(3-hydroxybutyl)-1-methyl-2-oxo-2,3-dihydro-1H-benzo[d]imidazol-5-yl)amino)nicotinonitrile; (S)-2-Chloro-4-((3-(3-hydroxybutyl)-1-methyl-2-oxo-2,3-dihydro-1H-benzo[d]imidazol-5-yl)amino)nicotinonitrile; 1-Methyl-5-nitro-1,3-dihydro-2H-benzo[d]imidazol-2-one; 2-Chloro-4-((1-methyl-2-oxo-2,3-dihydro-1H-benzo[d]-imidazol-5-yl)amino)nicotinonitrile; 3-(2-Hydroxybutyl)-1-methyl-5-nitro-1,3-dihydro-2Hbenzo[d]imidazol-2-one; 3-Hydroxy-3-methylbutyl 4-methylbenzenesulfonate; [(3R)-3-Hydroxybutyl] 4-methylbenzenesulfonate; 3-(3-Hydroxy-3-methylbutyl)-1-methyl-5-nitro-1,3-dihydro-2H-benzo[d]imidazol-2-one; 5-Amino-3-(3-hydroxy-3-methylbutyl)-1-methyl-1,3-dihydro-2H-benzo[d]imidazol-2-one; 5-((2-Bromo-5-chloropyridin-4-yl)amino)-3-(3-hydroxy-3-methylbutyl)-1-methyl-1,3-dihydro-2H-benzo[d]imidazol-2-one; 5-Chloro-2-((3S,5R)-3,5-dimethylpiperidin-1-yl)-4-iodopyridine; (2S,6R)-4-(5-Chloro-4-iodopyridin-2-yl)-2,6-dimethylmorpholine; and various combinations thereof. In certain embodiments, the compound is BI-3802 or an analog thereof.

In some embodiments, degradation is effected by an E3 ubiquitin ligase.

In various embodiments, polymerization is reversed by a BCL6 inhibitor binding to the BTB domain. In some embodiments, the BCL6 inhibitor is a quinolinone. In some embodiments, the BCL6 inhibitor is a benzimidazolone compound. In certain embodiments, the BCL6 inhibitor is selected from the group consisting of BI-3812 (1-(5-chloro-4-((8-methoxy-1-methyl-3-(2-(methylamino)-2-oxoethoxy)-2-oxo-1,2-dihydroquinolin-6-yl)amino)pyrimidin-2-yl)-N,N-dimethylpiperidine-4-carboxamide); 5-((5-Chloro-2-(2,4-dimethylthiazol-5-yl)pyrimidin-4-yl)-amino)-3-(3-hydroxy-3-methylbutyl)-1-methyl-1,3-dihydro-2H-benzo[d]imidazol-2-one; 5-((5-Chloro-2-(1-methyl-1H-imidazol-2-yl)pyrimidin-4-yl)-amino)-3-(3-hydroxy-3-methylbutyl)-1-methyl-1,3-dihydro-2H-benzo[d]imidazol-2-one; 5-((5-Chloro-2-(1H-pyrazol-1-yl)pyrimidin-4-yl)amino)-3-(3-hydroxy-3-methylbutyl)-1-methyl-1,3-dihydro-2H-benzo[d]-imidazol-2-one; 5-((5-Chloro-2-(3-methyl-1H-pyrazol-1-yl)pyrimidin-4-yl)-amino)-3-(3-hydroxy-3-methylbutyl)-1-methyl-1,3-dihydro-2H-benzo[d]imidazol-2-one; 5-((5-Chloro-2-(5-methyl-1H-pyrazol-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(3,5-dimethyl-1H-pyrazol-1-yl)pyrimidin-4-yl)amino)-3-(3-hydroxy-3-methylbutyl)-1-methyl-1,3-dihydro-2H-benzo[d]imidazol-2-one; 5-((5-Chloro-2-((2R,6S)-2,6-dimethylmorpholino)pyrimidin-4-yl)amino)-3-(3-hydroxy-3-methylbutyl)-1-methyl-1,3-dihydro-2H-benzo[d]imidazol-2-one; 5-((5-Chloro-2-(2,2,6,6-tetramethylmorpholino)pyrimidin-4-yl)amino)-3-(3-hydroxy-3-methylbutyl)-1-methyl-1,3-dihydro-2H-benzo[d]imidazol-2-one; 5-((5-Chloro-2-((3S,5R)-3,4,5-trimethylpiperazin-1-yl)-pyrimidin-4-yl)amino)-3-(3-hydroxy-3-methylbutyl)-1-methyl-1,3-dihydro-2H-benzo[d]imidazol-2-one; 5-((5-Chloro-2-(3,5-dimethyl-1H-pyrazol-1-yl)pyridin-4-yl)-amino)-3-(3-hydroxy-3-methylbutyl)-1-methyl-1,3-dihydro-2H-benzo[d]imidazol-2-one; 1-(5-Chloro-4-((3-(3-hydroxy-3-methylbutyl)-1-methyl-2-oxo-2,3-dihydro-1H-benzo[d]imidazol-5-yl)amino)pyrimidin-2-yl)piperidine-3-carbonitrile; 5-((5-Chloro-2-(4-(trifluoromethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(8,8-difluoro-3-azabicyclo[3.2.1]octan-3-yl)-pyrimidin-4-yl)amino)-3-(3-hydroxy-3-methylbutyl)-1-methyl-1,3-dihydro-2H-benzo[d]imidazol-2-one; 5-((5-Chloro-2-(3,3-difluoro-8-azabicyclo[3.2.1]octan-8-yl)-pyrimidin-4-yl)amino)-3-(3-hydroxy-3-methylbutyl)-1-methyl-1,3-dihydro-2H-benzo[d]imidazol-2-one; 5-((5-Chloro-2-(dimethylamino)pyrimidin-4-yl)amino)-3-(3-hydroxy-3-methylbutyl)-1-methyl-1,3-dihydro-2H-benzo[d]-imidazol-2-one; 5-((5-Chloro-2-morpholinopyrimidin-4-yl)amino)-3-(3-hydroxy-3-methylbutyl)-1-methyl-1,3-dihydro-2H-benzo[d]-imidazol-2-one; 5-((5-Chloro-2-(piperidin-1-yl)pyrimidin-4-yl)amino)-3-(3-hydroxy-3-methylbutyl)-1-methyl-1,3-dihydro-2H-benzo[d]-imidazol-2-one; 5-((5-Chloro-2-((2R,6S)-2,6-dimethylmorpholino)pyridin-4-yl)amino)-3-(3-hydroxy-3-methylbutyl)-1-methyl-1,3-dihydro-2H-benzo[d]imidazol-2-one; 2-Chloro-4-((3-(3-hydroxy-3-methylbutyl)-1-methyl-2-oxo-2,3-dihydro-1H-benzo[d]imidazol-5-yl)amino)nicotinonitrile; 5-((2,3-Dichloropyridin-4-yl)amino)-3-(3-hydroxy-3-methylbutyl)-1-methyl-1,3-dihydro-2H-benzo[d]imidazol-2-one; 5-((3-Chloropyridin-4-yl)amino)-3-(3-hydroxy-3-methylbutyl)-1-methyl-1,3-dihydro-2H-benzo[d]imidazol-2-one; 4-Chloro-6-((3-(3-hydroxy-3-methylbutyl)-1-methyl-2-oxo-2,3-dihydro-1H-benzo[d]imidazol-5-yl)amino)pyrimidine-5-Carbonitrile; 5-((5,6-Dichloropyrimidin-4-yl)amino)-3-(3-hydroxy-3-methylbutyl)-1-methyl-1,3-dihydro-2H-benzo[d]imidazol-2-one; 5-((3,5-Dichloropyridin-4-yl)amino)-3-(3-hydroxy-3-methylbutyl)-1-methyl-1,3-dihydro-2H-benzo[d]imidazol-2-one; 5-((5-Chloro-2-(methylthio)pyrimidin-4-yl)amino)-3-(3-hydroxy-3-methylbutyl)-1-methyl-1,3-dihydro-2H-benzo[d]-imidazol-2-one; 5-((2,5-Dichloropyrimidin-4-yl)amino)-3-(3-hydroxy-3-methylbutyl)-1-methyl-1,3-dihydro-2H-benzo[d]imidazol-2-one; 2-Chloro-4-((cyclopropylmethyl)amino)nicotinonitrile; 3,4,2-Chloro-4-((1,3-dimethyl-2-oxo-2,3-dihydro-1H-benzo[d]-imidazol-5-yl)amino)nicotinonitrile; 2-Chloro-4-((3-(2-hydroxybutyl)-1-methyl-2-oxo-2,3-dihydro-1H-benzo[d]imidazol-5-yl)amino)nicotinonitrile; 2-Chloro-4-((3-(2-cyanobutyl)-1-methyl-2-oxo-2,3-dihydro-1H-benzo[d]imidazol-5-yl)amino)nicotinonitrile; (S)-2-Chloro-4-((3-(2-hydroxybutyl)-1-methyl-2-oxo-2,3-dihydro-1H-benzo[d]imidazol-5-yl)amino)nicotinonitrile; (R)-2-Chloro-4-((3-(2-hydroxybutyl)-1-methyl-2-oxo-2,3-dihydro-1H-benzo[d]imidazol-5-yl)amino)nicotinonitrile; 4-((3-Butyl-1-methyl-2-oxo-2,3-dihydro-1H-benzo[d]-imidazol-5-yl)amino)-2-chloronicotinonitrile; (R)-2-Chloro-4-((3-(3-hydroxybutyl)-1-methyl-2-oxo-2,3-dihydro-1H-benzo[d]imidazol-5-yl)amino)nicotinonitrile; (S)-2-Chloro-4-((3-(3-hydroxybutyl)-1-methyl-2-oxo-2,3-dihydro-1H-benzo[d]imidazol-5-yl)amino)nicotinonitrile; 1-Methyl-5-nitro-1,3-dihydro-2H-benzo[d]imidazol-2-one; 2-Chloro-4-((1-methyl-2-oxo-2,3-dihydro-1H-benzo[d]-imidazol-5-yl)amino)nicotinonitrile; 3-(2-Hydroxybutyl)-1-methyl-5-nitro-1,3-dihydro-2Hbenzo[d]imidazol-2-one; 3-Hydroxy-3-methylbutyl 4-methylbenzenesulfonate; [(3R)-3-Hydroxybutyl] 4-methylbenzenesulfonate; 3-(3-Hydroxy-3-methylbutyl)-1-methyl-5-nitro-1,3-dihydro-2H-benzo[d]imidazol-2-one; 5-Amino-3-(3-hydroxy-3-methylbutyl)-1-methyl-1,3-dihydro-2H-benzo[d]imidazol-2-one; 5-((2-Bromo-5-chloropyridin-4-yl)amino)-3-(3-hydroxy-3-methylbutyl)-1-methyl-1,3-dihydro-2H-benzo[d]imidazol-2-one; 5-Chloro-2-((3S,5R)-3,5-dimethylpiperidin-1-yl)-4-iodopyridine; (2S,6R)-4-(5-Chloro-4-iodopyridin-2-yl)-2,6-dimethylmorpholine; and various combinations thereof. In certain embodiments, the BCL6 inhibitor is BI-3812 or an analog thereof.

In some embodiments, the heterologous amino acid sequence is operably linked to an N-terminus or to a C-terminus of the fusion polypeptide. In some embodiments, the heterologous amino acid sequence includes at least a fragment of a transcription factor, where the transcription factor has DNA regulatory activity. In some embodiments, the transcription factor is BCL6. In various embodiments, the heterologous amino acid sequence includes at least a fragment of an oncogene. In various embodiments, the heterologous amino acid sequence includes at least a fragment of an enzyme having enzymatic activity or a signaling polypeptide having signal transduction activity.

In one aspect, the invention generally features a nucleotide molecule encoding the recombinant polypeptide or the fusion polypeptide. In some embodiments, the nucleotide molecule includes DNA or RNA.

In one aspect, the invention features an expression vector including the nucleotide molecule. In some embodiments, the expression vector including a promoter sequence. In various embodiments, the expression vector is a mammalian expression vector. In some embodiments, the expression vector is a viral vector. In various embodiments, the viral vector is a lentiviral, baculoviral, adenoviral, or an adeno-associated virus vector.

In another aspect, the invention features a cell including the nucleotide molecule, or the expression vector. In some embodiments, the cell is a mammalian cell. In various embodiments, the cell is a human cell. In certain embodiments, the cell is a COS7, CHO, 293T, Hela, a HEK293 cell, a SuDHL4 cell, a Raji cell, a DEL cell, a HEK293T cell, a T cell, a B cell, or a DG44 cell or Vero cell. In certain embodiments, the cell is a cancer cell.

In a further aspect, the invention features a method for inducing polymerization of a polypeptide of interest, the method involving operably linking the polypeptide of interest to a BTB domain to generate a fusion polypeptide, and contacting the fusion polypeptide with a compound that induces polymerization of the BTB domain, thereby inducing polymerization of the fusion polypeptide.

In one aspect, the invention generally features a method for increasing the rate of degradation of a polypeptide of interest, the method involving operably linking the polypeptide of interest to a BTB domain to generate a fusion polypeptide, and contacting the fusion polypeptide with a compound that induces polymerization of the BTB domain, thereby inducing polymerization of the fusion polypeptide, where polymerization of the fusion polypeptide enhances degradation of the fusion polypeptide.

In a further aspect, the invention features a method for reversing polymerization of a polypeptide, the method involving contacting a fusion polypeptide with a BCL6 inhibitor after the fusion polypeptide has been polymerized by the method of claim 47, where contacting the fusion polypeptide with the BCL6 inhibitor causes the BTB domain to depolymerize, thereby reversing polymerization of the polypeptide.

In some embodiments, the compound is BI-3802 or an analog thereof.

In various embodiments, the method is carried out in in vitro or in a cell.

In some embodiments, the BCL6 inhibitor is BI-3812 or an analog thereof.

In some embodiments, the polymerization localizes the fusion polypeptide to foci within the cell. In various embodiments, the polypeptide of interest including an enzyme. In some embodiments, localization increases the concentration of the enzyme within a portion of the cell.

In some embodiments, activity of the enzyme is enhanced or altered by the polymerization.

In another aspect, the invention provides a method for measuring efficacy of an agent in inducing degradation of a polypeptide including a BTB domain and expressed by a cell, the method involving (a) contacting a cell expressing a detectable fusion polypeptide including the BTB domain with an agent to be evaluated for inducing degradation of the fusion polypeptide; and (b) detecting the level of fusion polypeptide present in the cell after being contacted with the agent relative to the level of fusion polypeptide present in a corresponding control cell, where a reduction in the level of fusion polypeptide identifies the agent as effective in inducing degradation of the polypeptide including the BTB domain.

In some embodiments, the fusion polypeptide is detected by monitoring fluorescence or by imaging. In some embodiments, the method is carried out in vivo or in vitro. In some embodiments, the fusion polypeptide includes a fluorescent polypeptide.

In an aspect, the invention provides a method for selecting a subject for treatment with a compound that induces degradation of a BCL6 polypeptide, the method involving detecting in a biological sample of the subject the presence or absence of an alteration at amino acid R28, E41, C84, G55, Y58 or BCL6, where the absence of the alteration selects the subject for treatment with a compound that induces degradation of a BCL6 polypeptide.

In an embodiment, the subject has a B-cell lymphoma cell, a Non-Hodgkin lymphoma cell, a diffuse large B cell lymphoma (DLBCL) cell, or a follicular lymphoma cell. In some embodiments, the compound that induces degradation of a BCL6 polypeptide is BI-3802 or an analog thereof.

In another aspect, the invention generally provides for a biomaterial prepared by a method involving operably linking a polypeptide of interest to a BTB domain to generate a fusion polypeptide, and contacting the fusion polypeptide with a compound that induces polymerization of the BTB domain, thereby inducing polymerization of the fusion polypeptide and formation of the biomaterial.

The invention provides BTB fusion polypeptides, and methods of using such polypeptides. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any compound, small molecule chemical compound, antibody, nucleic acid molecule, or polypeptide, peptide, or fragments thereof.

By “alteration” is meant a change (increase or decrease) in the expression levels, activity, or structure of a gene or polypeptide, or in an intracellular concentrations of a polypeptide as detected by standard art known methods such as those described herein. As used herein, an alteration includes a 10% change in expression levels of a gene or polypeptide or in intracellular concentrations of a polypeptide, preferably a 25% change, more preferably a 40% change, and most preferably a 50% or greater change in expression levels. By “alter” is meant to effect an alteration.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. As another example, an analog of a compound (e.g., a small molecule compound) retains the biological activity of the compound (e.g., inducing, inhibiting, or reversing polymerization of a polypeptide).

By “BCL6 (B-cell lymphoma 6) polypeptide” is meant a polypeptide or fragment thereof having at least 85% amino acid sequence identity to NCBI Accession No. NP_001124317, which is reproduced below, and comprises a Broad complex/Tramtrack/Bric-a-brac (BTB) domain amino acid sequence capable of undergoing reversible polymerization, where the polymerization is optionally induced by a compound and where the polymerization is optionally reversed by a BCL6 inhibitor. In various embodiments, the compound is BI-3802 and the BCL6 inhibitor is BI-3812. NCBI Accession No. NP_001124317 is reproduced below:

MASPADSCIQ FTRHASDVLL NLNRLRSRDI LTDVVIVVSR
EQFRAHKTVL MACSGLFYSI FTDQLKCNLS VINLDPEINP
EGFCILLDFM YTSRLNLREG NIMAVMATAM YLQMEHVVDT
CRKFIKASEA EMVSAIKPPR EEFLNSRMLM PQDIMAYRGR
EVVENNLPLR SAPGCESRAF APSLYSGLST PPASYSMYSH
LPVSSLLFSD EEFRDVRMPV ANPFPKERAL PCDSARPVPG
EYSRPTLEVS PNVCHSNIYS PKETIPEEAR SDMHYSVAEG
LKPAAPSARN APYFPCDKAS KEEERPSSED EIALHFEPPN
APLNRKGLVS PQSPQKSDCQ PNSPTESCSS KNACILQASG
SPPAKSPTDP KACNWKKYKF IVLNSLNQNA KPEGPEQAEL
GRLSPRAYTA PPACQPPMEP ENLDLQSPTK LSASGEDSTI
PQASRLNNIV NRSMTGSPRS SSESHSPLYM HPPKCTSCGS
QSPQHAEMCL HTAGPTFPEE MGETQSEYSD SSCENGAFFC
NECDCRFSEE ASLKRHTLQT HSDKPYKCDR CQASFRYKGN
LASHKTVHTG EKPYRCNICG AQFNRPANLK THTRIHSGEK
PYKCETCGAR FVQVAHLRAH VLIHTGEKPY PCEICGTRFR
HLQTLKSHLR IHTGEKPYHC EKCNLHFRHK SQLRLHLRQK
HGAITNTKVQ YRVSATDLPP ELPKAC.

By “nucleotide molecule encoding a BCL6 (B-cell lymphoma) polynucleotide” is meant a nucleotide molecule or fragment thereof having at least 85% amino acid sequence identity to NCBI Reference Sequence No. NM_001130845, which is reproduced below, and encodes a BCL6 polypeptide comprising a broad complex/Tramtrack/Bric-a-brac (BTB) domain capable of undergoing reversible polymerization, where the polymerization is optionally induced by a compound and where the polymerization is optionally reversed by a BCL6 inhibitor. In various embodiments, the compound is BI-3802 and the BCL6 inhibitor is BI-3812. NCBI Accession No. NM_001130845 is reproduced below:

ACAAGCGAGC TGGTGGTTGA AGCTGGTTAA AGAACAGCCT
AGGTATTCCA GAAGTGTTTG AGGATCCCTT CCATGAAGGA
AGAGAGGAAA GTTTTTAAGT AAACCTCCCA CTCCCATGTG
TCTTCAGCTT TCTTTTGCAA AGGAGAAAAT CCTTGAAGTT
TGGTAAAGAC CGAGTTAGTC TATCTCTCTT TGCCTATCTC
GAGTTGGGCT GGGGAGAGGA GGAGATAGGT TCTTTTGTCT
TTTTCTGTCT TCTCCCTTCC CCACTTCCTT CCCTCCAGTC
CCCACTCACT CACATGCACA CACTAACCTT GGAGCCGATG
GGATTGAGTG ACTGGCACTT GGGACCACAG AGAAATGTCA
GAGTGTTTGG TTACAGACTC AAGGAAACCT CTCATTTTAG
AGTGCTCATT TGGTTTTGAG CAAAATTTTG GACTGTGAAG
CAAGGCATTG GTGAAGACAA AATGGCCTCG CCGGCTGACA
GCTGTATCCA GTTCACCCGC CATGCCAGTG ATGTTCTTCT
CAACCTTAAT CGTCTCCGGA GTCGAGACAT CTTGACTGAT
GTTGTCATTG TTGTGAGCCG TGAGCAGTTT AGAGCCCATA
AAACGGTCCT CATGGCCTGC AGTGGCCTGT TCTATAGCAT
CTTTACAGAC CAGTTGAAAT GCAACCTTAG TGTGATCAAT
CTAGATCCTG AGATCAACCC TGAGGGATTC TGCATCCTCC
TGGACTTCAT GTACACATCT CGGCTCAATT TGCGGGAGGG
CAACATCATG GCTGTGATGG CCACGGCTAT GTACCTGCAG
ATGGAGCATG TTGTGGACAC TTGCCGGAAG TTTATTAAGG
CCAGTGAAGC AGAGATGGTT TCTGCCATCA AGCCTCCTCG
TGAAGAGTTC CTCAACAGCC GGATGCTGAT GCCCCAAGAC
ATCATGGCCT ATCGGGGTCG TGAGGTGGTG GAGAACAACC
TGCCACTGAG GAGCGCCCCT GGGTGTGAGA GCAGAGCCTT
TGCCCCCAGC CTGTACAGTG GCCTGTCCAC ACCGCCAGCC
TCTTATTCCA TGTACAGCCA CCTCCCTGTC AGCAGCCTCC
TCTTCTCCGA TGAGGAGTTT CGGGATGTCC GGATGCCTGT
GGCCAACCCC TTCCCCAAGG AGCGGGCACT CCCATGTGAT
AGTGCCAGGC CAGTCCCTGG TGAGTACAGC CGGCCGACTT
TGGAGGTGTC CCCCAATGTG TGCCACAGCA ATATCTATTC
ACCCAAGGAA ACAATCCCAG AAGAGGCACG AAGTGATATG
CACTACAGTG TGGCTGAGGG CCTCAAACCT GCTGCCCCCT
CAGCCCGAAA TGCCCCCTAC TTCCCTTGTG ACAAGGCCAG
CAAAGAAGAA GAGAGACCCT CCTCGGAAGA TGAGATTGCC
CTGCATTTCG AGCCCCCCAA TGCACCCCTG AACCGGAAGG
GTCTGGTTAG TCCACAGAGC CCCCAGAAAT CTGACTGCCA
GCCCAACTCG CCCACAGAGT CCTGCAGCAG TAAGAATGCC
TGCATCCTCC AGGCTTCTGG CTCCCCTCCA GCCAAGAGCC
CCACTGACCC CAAAGCCTGC AACTGGAAGA AATACAAGTT
CATCGTGCTC AACAGCCTCA ACCAGAATGC CAAACCAGAG
GGGCCTGAGC AGGCTGAGCT GGGCCGCCTT TCCCCACGAG
CCTACACGGC CCCACCTGCC TGCCAGCCAC CCATGGAGCC
TGAGAACCTT GACCTCCAGT CCCCAACCAA GCTGAGTGCC
AGCGGGGAGG ACTCCACCAT CCCACAAGCC AGCCGGCTCA
ATAACATCGT TAACAGGTCC ATGACGGGCT CTCCCCGCAG
CAGCAGCGAG AGCCACTCAC CACTCTACAT GCACCCCCCG
AAGTGCACGT CCTGCGGCTC TCAGTCCCCA CAGCATGCAG
AGATGTGCCT CCACACCGCT GGCCCCACGT TCCCTGAGGA
GATGGGAGAG ACCCAGTCTG AGTACTCAGA TTCTAGCTGT
GAGAACGGGG CCTTCTTCTG CAATGAGTGT GACTGCCGCT
TCTCTGAGGA GGCCTCACTC AAGAGGCACA CGCTGCAGAC
CCACAGTGAC AAACCCTACA AGTGTGACCG CTGCCAGGCC
TCCTTCCGCT ACAAGGGCAA CCTCGCCAGC CACAAGACCG
TCCATACCGG TGAGAAACCC TATCGTTGCA ACATCTGTGG
GGCCCAGTTC AACCGGCCAG CCAACCTGAA AACCCACACT
CGAATTCACT CTGGAGAGAA GCCCTACAAA TGCGAAACCT
GCGGAGCCAG ATTTGTACAG GTGGCCCACC TCCGTGCCCA
TGTGCTTATC CACACTGGTG AGAAGCCCTA TCCCTGTGAA
ATCTGTGGCA CCCGTTTCCG GCACCTTCAG ACTCTGAAGA
GCCACCTGCG AATCCACACA GGAGAGAAAC CTTACCATTG
TGAGAAGTGT AACCTGCATT TCCGTCACAA AAGCCAGCTG
CGACTTCACT TGCGCCAGAA GCATGGCGCC ATCACCAACA
CCAAGGTGCA ATACCGCGTG TCAGCCACTG ACCTGCCTCC
GGAGCTCCCC AAAGCCTGCT GAAGCATGGA GTGTTGATGC
TTTCGTCTCC AGCCCCTTCT CAGAATCTAC CCAAAGGATA
CTGTAACACT TTACAATGTT CATCCCATGA TGTAGTGCCT
CTTTCATCCA CTAGTGCAAA TCATAGCTGG GGGTTGGGGG
TGGTGGGGGT CGGGGCCTGG GGGACTGGGA GCCGCAGCAG
CTCCCCCTCC CCCACTGCCA TAAAACATTA AGAAAATCAT
ATTGCTTCTT CTCCTATGTG TAAGGTGAAC CATGTCAGCA
AAAAGCAAAA TCATTTTATA TGTCAAAGCA GGGGAGTATG
CAAAAGTTCT GACTTGACTT TAGTCTGCAA AATGAGGAAT
GTATATGTTT TGTGGGAACA GATGTTTCTT TTGTATGTAA
ATGTGCATTC TTTTAAAAGA CAAGACTTCA GTATGTTGTC
AAAGAGAGGG CTTTAATTTT TTTAACCAAA GGTGAAGGAA
TATATGGCAG AGTTGTAAAT ATATAAATAT ATATATATAT
AAAATAAATA TATATAAACC TAAAAAAGAT ATATTAAAAA
TATAAAACTG CGTTAAAGGC TCGATTTTGT ATCTGCAGGC
AGACACGGAT CTGAGAATCT TTATTGAGAA AGAGCACTTA
AGAGAATATT TTAAGTATTG CATCTGTATA AGTAAGAAAA
TATTTTGTCT AAAATGCCTC AGTGTATTTG TATTTTTTTG
CAAGTGAAGG TTTACAATTT ACAAAGTGTG TATTAAAAAA
AACAAAAAGA ACAAAAAAAT CTGCAGAAGG AAAAATGTGT
AATTTTGTTC TAGTTTTCAG TTTGTATATA CCCGTACAAC
GTGTCCTCAC GGTGCCTTTT TTCACGGAAG TTTTCAATGA
TGGGCGAGCG TGCACCATCC CTTTTTGAAG TGTAGGCAGA
CACAGGGACT TGAAGTTGTT ACTAACTAAA CTCTCTTTGG
GAATGTTTGT CTCATCCCAT TCTGCGTCAT GCTTGTGTTA
TAACTACTCC GGAGACAGGG TTTGGCTGTG TCTAAACTGC
ATTACCGCGT TGTAAAATAT AGCTGTACAA ATATAAGAAT
AAAATGTTGA AAAGTCAAA.

By “Broad complex/Tramtrack/Bric-a-brac (BTB) domain” is meant domain having at least about 85% amino acid sequence identity to a BCL6 BTB domain comprising amino acids 1-129 or a fragment thereof that mediates compound binding, polymerization, or degradation.

Non-limiting examples of Broad complex/Tramtrack/Bric-a-brac (BTB) domains suitable for use in various embodiments of the invention of the disclosure are described by Stogios, P J, et al. “Sequence and structural analysis of BTB domain proteins,” Genome Biology 6:R82 (2005), which is incorporated herein by reference for all purposes. Further incorporated herein by reference are the over 2,200 non-redundant BTB domain sequences available in the database referenced in Stogios, P J, et al. “Sequence and structural analysis of BTB domain proteins,” Genome Biology 6:R82 (2005). In various embodiments, the Broad complex/Tramtrack/Bric-a-brac domain is the Broad complex/Tramtrack/Bric-a-brac domain of the B-cell lymphoma 6 (BCL6) polypeptide, optionally comprising about amino acid residues 1, 2, 3, 4, 5, 10, 14, 15, 20, 21, 22, 25, 50, 75, 90, 95, or 100 to about residue 100, 110, 120, 121, 122, 123, 124, 125, 125, 127, 128, 129, 130, 140, 150, 160, 170, 180, 190, 200, 210, 220, 230, 240, 245, 249, 250, 251, 260, 270, 275, 280, 300, 325, 350, 360, 370, 380, 382, 385, or 390 of the B-cell lymphoma 6 polypeptide. The Broad complex/Tramtrack/Bric-a-brac domain can be derived from BCL6.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “degron” is meant a portion of a polypeptide that regulates polypeptide degradation. In various embodiments, a degron is a short amino acid sequence, a structural motif, or exposed amino acids (e.g., lysine or arginine) located in the polypeptide. In some embodiments, a degron has the sequence V×P (Val-x-Pro), where x is any amino acid. In some embodiments, a degron can mediate interactions with an E3 ubiquitin ligase (e.g., E3 ubiquitin-protein ligase seven in absentia homolog 1 (SIAH1) or E3 ubiquitin ligase seven in absentia homolog 2 (SIAH2)). In some embodiments, V×P is recognized by seven in absentia homolog 1 (SIAH1). In some embodiments, the degron corresponds to amino acid residues 249-251 of NCBI Accession No. NP_001124317. In some embodiments, the degron comprises the V×P E3 ubiquitin-protein ligase seven in absentia homolog 1 (SIAH1) binding motif depicted in FIG. 9B. In some embodiments, the degron comprises amino acid residues 241-260 of BCL6. In some embodiments, the degron comprises any one of the individual V×P E3 ubiquitin-protein ligase seven in absentia homolog 1 (SIAH1) binding motifs listed in FIG. 9B. The degron can comprise about or at least 5, 10, 15, 20, 25, 30, 45, or 50 amino acid residues. In some embodiments, the degron comprises no more than about 5, 10, 15, 20, 25, 30, 45, or 50 amino acid residues.

“Detect” or “image” refers to identifying the presence, absence or amount of an analyte to be detected. In some embodiments, the analyte is foci comprising polymerized polypeptides. In some embodiments, the analyte is a detectable label.

By “detectable label” is meant a composition (e.g., a fluorophore, a peptide tag, an antigen, or a fluorescing polypeptide) that when linked to a molecule (e.g., a polypeptide or polypeptide domain) of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, fluorescent polypeptides (e.g., green fluorescent protein, enhanced green fluorescent protein (eGFP), red fluorescent protein, mCherry, etc.), or haptens.

By “disease” is meant any condition or disorder that damages or interferes with the normal function of a cell, tissue, or organ. Non-limiting examples of diseases include autoimmune diseases, cancers, or tumors. Non-limiting examples of cancers or tumors include B-cell lymphoma, Non-Hodgkin lymphoma, diffuse large B-cell lymphoma (DLBCL), and follicular lymphomas, multiple myeloma, acute myeloid leukemia, leukemia, and Hodgkin lymphoma. In some embodiments, the autoimmune disease is type 1 diabetes, rheumatoid arthritis, psoriasis, multiple sclerosis, systemic lupus erythematosus, grave's disease, sjogren's syndrome, hashimoto's thyroiditis, myasthenia gravis, autoimmune vasculitis, pernicious anemia, or celiac disease. Further non-limiting examples of cancers include adrenal cancer, anal cancer, appendix cancer, bile duct cancer, bladder cancer, bone cancer, brain cancer, breast cancer, cervical cancer, colorectal cancer esophageal cancer, gallbladder cancer, mesothelioma, neuroendocrine tumors, oral cancer, ovarian cancer, pancreatic cancer, prostate cancer, sinus cancer, skin cancer, soft tissue sarcoma, gestational trophoblastic disease, spinal cancer, head and neck cancer, stomach cancer, testicular cancer, intestinal cancer, throat cancer, kidney cancer, thyroid cancer, uterine cancer, liver cancer, vaginal cancer, lung cancer, or vulvar cancer.

By “enhance” is meant to effect an increase in a measurable quantity; for example, to effect an increase in enzyme kinetics, enzyme activity, degradation rates, or signaling rates. In various embodiments, to enhance a measurable quantity results in an increase in the quantity of about or at least about 5%, 10%, 15%, 20%, 30%, 40%, 50%, 75%, 100%, 250%, 500%, or 1,000% relative to a value of the measurable quantity prior to being enhanced. In various embodiments, polymerization of a polypeptide induced by a compound effects an increase in the measurable quantity.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “foci” is meant a collection of discrete regions comprising high local concentration of a polypeptide. In various embodiments, foci comprise polymers comprising the polypeptide. In some embodiments, the foci can be imaged using techniques in microscopy (e.g., immunofluorescence microscopy, fluorescence-activated cell sorting, imaging flow cytometry, or fluorescence microscopy). Examples of imaged foci are provided throughout the Figures provided herein, see for example FIG. 1F.

By “fragment” is meant a portion of a polypeptide or nucleic acid molecule. In some embodiments, this portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “heterologous,” or “exogenous” is meant a peptide, nucleotide molecule, polypeptide or nucleotide sequence, or polypeptide that 1) has been experimentally placed into a cell that does not normally comprise the peptide, nucleotide molecule, polypeptide or nucleotide sequence, or polypeptide or that 2) has been experimentally linked to a polypeptide sequence or nucleotide sequence to which the a peptide, nucleotide molecule, polypeptide or nucleotide sequence, or polypeptide is not normally linked to in nature. In some embodiments, “heterologous” means that a peptide, nucleotide molecule, polypeptide or nucleotide sequence, or polypeptide has been experimentally placed into a non-native context; for example, to say that a heterologous polypeptide sequence has been linked to a polypeptide domain means that the polypeptide domain having a first sequence and the heterologous polypeptide sequence having a second sequence are not found linked together in nature to constitute a polypeptide comprising the first sequence and the second sequence. In some embodiments, the heterologous polypeptide, sequence, or polypeptide is derived from a first polypeptide, optionally from a first species or host organism, and is linked to a second polypeptide, sequence, or polypeptide derived from a second polypeptide, optionally from a second species or host organism. In some embodiments, the first species or host organism is different from the second species or host organism.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “indication” is meant a particular disease treated by administering an agent.

By “inhibitor” is meant an agent that prevents or reverses polymerization or oligomerization of a polypeptide. In some embodiments, an inhibitor competes for or prevents binding of a compound to a polypeptide. In some embodiments, an inhibitor is an agent that reduces the degradation rate of a polypeptide or reduces enhancement of degradation, polymerization, or enzymatic activity effected by a compound.

By “induce” is meant to initiate an enhancement or change, e.g. to initiate polymerization of a polypeptide. For example, a compound induces polymerization of a polypeptide if the polypeptide undergoes polymerization in response to being contacted with the compound or if polymerization of the polypeptide is enhanced when the polypeptide is contacted by the compound.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of this invention is purified if it is substantially free of cellular material, viral material, or culture medium when produced by recombinant DNA techniques, or chemical precursors or other chemicals when chemically synthesized. Purity and homogeneity can be determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated recombinant polynucleotide” is meant a nucleic acid (e.g., a DNA or RNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the polypeptide. Purity can be measured by any appropriate method, for example, column chromatography, polyacrylamide gel electrophoresis, or by HPLC analysis.

By “linker” is meant a molecule used to connect two molecules to each other wherein one molecule is attached to one end of the linker and the other molecule is attached to the other end of the liner. In some embodiments, the linker is a polypeptide to which one molecule can be covalently linked at the C-terminus of the linker and another molecule can be covalently linked to the N-terminus of the linker. In some embodiments, the linker is a polynucleotide.

By “marker” is meant any polypeptide or polynucleotide having an alteration in expression level or activity that is associated with a disease, disorder, metabolic state, biochemical condition, or intracellular condition.

As used herein, “obtaining” as in “obtaining an agent” includes synthesizing, purchasing, or otherwise acquiring the agent.

By the term “operably linked” is meant a fusion or bond or an association, of sufficient stability for the purposes of the invention. In some embodiments, two polypeptide sequences may be operably linked one to the other by a third polypeptide sequence. In some embodiments, the third polypeptide sequence may comprise a linker, a degron, or a combination thereof.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition. In some embodiments, a reference is a control condition measured prior to administration of an agent to a cell or to a subject or prior to making a change in an amount of the agent administered to the cell or subject.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “resistant” is meant failure or inability to respond in an advantageous or therapeutic manner to an agent. As a non-limiting example, a cancer or tumor cell resistant to treatment with an agent is a cancer or tumor cell that continues to proliferate when administered the agent. As a further non-limiting example, a cancer patient is resistant to treatment with an agent if the agent is ineffective in reducing, abating, or eliminating a population of cancer cells in the patient. As another non-limiting example, a cancer or tumor cell is resistant to an agent if viability of the cancer or tumor cell is not adversely impacted when the cancer or tumor cell is contacted with the agent.

By “sensitive” is meant the capacity or demonstrated capacity to respond in an advantageous or therapeutic manner to an agent. As a non-limiting example, a cancer or tumor cell sensitive to treatment with an agent is a cancer or tumor cell that fails to proliferate, shows a reduction in proliferation rate, or undergoes apoptosis when administered the agent. As a further non-limiting example, a cancer patient is sensitive to treatment with an agent if the agent is effective in reducing, abating, or eliminating a population of cancer cells in the patient. As another non-limiting example, a cancer or tumor cell is sensitive to an agent if viability of the cancer or tumor cell is adversely impacted or reduced when the cancer or tumor cell is contacted with the agent.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule comprising nucleotides that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). In some embodiments, nucleic acid molecules can comprise modified nucleotides such as, to provide non-limiting examples, locked nucleic acids (LNA), DNA, RNA, or various combinations thereof.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “peptide” is meant a polypeptide comprising from about 2 to about 50 amino acids.

By “polymerize” is meant the process by which a plurality of polypeptide molecules (e.g., fusion polypeptides) become bound to one another by covalent or non-covalent bonds to form a defined, crystalline, repeating, or regular structure, optionally a helical structure. The structure resulting from polymerization is a polymer. In various embodiments, the covalent or non-covalent bonds occur within a domain (e.g., a Broad complex/Tramtrack/Bric-a-brac domain) comprised by the polypeptide molecules such that the domain mediates polymerization. Polymerization is distinct from aggregation in that by “aggregation” is meant a process by which a plurality of polypeptide molecules become bound to one another by covalent or non-covalent bonds to form a non-crystalline, or otherwise ill-defined, or irregular structure. In various embodiments, “polymerization” results in the plurality of polypeptide molecules associating with one another in a linear arrangement, optionally forming filaments. In some embodiments, the linear arrangement of polypeptide molecules forms a helical structure. In various embodiments, the polypeptide molecules become bound to one another by non-covalent bonds. In some embodiments, the polypeptide molecules are fusion polypeptides each comprising a domain that mediates polymerization. Polymerization is distinct from oligomerization in that by “oligomerization” is meant a polymerization resulting in the association of not more than about 2, 10, 50, 75, or 100 polypeptide molecules.

By “polypeptide” is meant a linear organic polymer consisting of at least two amino-acid residues covalently linked together in a chain by one or more peptide bonds.

By “protein” is meant a polypeptide comprising at least about 50 amino acids. In some embodiments, a “protein” comprises not more than about 100, 150, 200, 250, 300, or 400 amino acid residues.

By “reversibly” or “reversible” when used to qualify “polymerization” or “oligomerization” is meant that a plurality of polypeptide molecules having undergone polymerization or oligomerization may be caused to dissociate from one another upon being exposed to an agent, optionally a small molecule. As a non-limiting example, a polymerization of a plurality of polypeptides is reversible if after having undergone polymerization the plurality of polypeptides depolymerize when contacted with an agent. In various embodiments, depolymerization results in dissociation of about or at least about 10%, 20%, 30%, 40%, 50%, 60%, 70%, 80%, 90% or 100% the plurality of polypeptides comprising a polymer from one another.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” or “patient” is meant a mammal, including, but not limited to, a human or non-human mammal, such as a bovine, equine, canine, ovine, or feline. In some embodiments, the subject is a human. The terms “subject” and “patient” are used interchangeably throughout the present disclosure.

Ranges provided herein (e.g. “at least” or “not more than” or “less than”) are understood to be shorthand for all of the values within the range. For example, a range of 0 to 50 or of at least 50 is understood to include any number, combination of numbers, or sub-range from the group consisting 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1G are chemical structures, a flow cytometry plot, bar graphs, schematics, and cell images demonstrating that BI-3802 treatment induces reversible BCL6 foci formation in vivo. FIG. 1A shows chemical structures of BI-3802 (a compound that enhances degradation of BCL6) and BI-3812 (a BCL6 inhibitor) with solvent exposed moieties circled. FIG. 1B is a flow cytometry plot. SuDHL4_{Cas9} cells were exposed to 1 μM BI-3802 for 4 hours followed by whole proteome quantification using tandem mass tag mass spectrometry (mean±old change, p-value calculated by a two-sided moderated t-test, n=3). FIG. 1C is a schematic of the BCL6 stability reporter: eGFP, enhanced green fluorescent protein; IRES, internal ribosome entry site. FIG. 1D is a bar graph. HEK293T_{Cas9} cells expressing the full length _{eGFP}BCL6^{FL} reporter were treated with DMSO, 0.5 μM MLN7243 (ubiquitin activating enzyme inhibitor), 5 μM MLN4924 (neddylation inhibitor) or 10 μM MG132 (26S proteasome inhibitor) for 3 hours and 1 μM BI-3812 or 1 μM BI-3802 for 1 hour. Bars represent mean±s.d. (n=3). FIG. 1E is a schematic representation of the BCL6 domain structures and a bar plot. HEK293T_{Cas9} cells expressing full length, mutant or truncated BCL6 fused to eGFP were exposed to DMSO or BI-3802 for 7 hours and fluorescence was measured by flow cytometry. Bars represent mean±s.d. (n=3). FIG. 1F provides images of cells. HEK293T_{Cas9} cells expressing the _{eGFP}BCL6¹⁻²⁷⁵ stability reporter were imaged following addition of DMSO or 1 μM BI-3802. FIG. 1G provides images of cells. HEK293T_{Cas9} cells expressing the _{eGFP}BCL6¹⁻²⁵⁰ stability reporter were imaged after treatment with DMSO or 1 μM BI-3802. After 75 minutes, 10 μM of BI-3812 was added where indicated.

FIGS. 2A-2C are chromatographs, electron micrographs, a computer-generated image, a class average image, a local resolution map, and cell images demonstrating that BI-3802 induces helical filament of BCL6 in vitro. FIG. 2A is a size-exclusion chromatogram of purified BCL6⁵⁻³⁶⁰ in the presence of DMSO, 2 μM BI-3812 or 2 μM BI-3802. FIG. 2B is a negative stain electron microscopy micrograph. BCL6⁵⁻³⁶⁰ protein was in the presence of DMSO, 2 μM BI-3802, or 20 μM BI-3802. Scale bars are 100 nm. FIG. 2C is a computer-generated image of a BCL6-BTB (AA1-129) filament constructed by extending the face-to-face model 2 (F2F_2), see FIG. 6, by RosettaDock.

FIGS. 3A-3D are plots, a bar graph, and a schematic showing that drug-induced BCL6 polymerization triggers rapid degradation by the ubiquitin-proteasome pathway. FIG. 3A is a plot. SuDHL4_{Cas9} cells expressing the BTB alanine variant library were treated with 1 μM BI-3802 or DMSO for 21 days. Mutations that conferred resistance were E41A, G55A, and C84A (over 1.6-fold enrichment, p-value <1e-02; n=4 codons/position; two-sided empirical rank-sum test-statistics). FIG. 3B presents plots. SuDHL4, Raji (both BCL6-dependent) and DEL (BCL6-independent) cells were infected with the indicated BCL6 variants and treated with 1 μM BI-3802 or DMSO over 21 days (n=3). FIG. 3C is a plot showing correlation of p-values for two genome-scale CRISPR-Cas9 knockout screens. For the reporter screen, HEK293T_{Cas9} cells expressing an _{eGFP}BCL6 reporter were treated with 1 μM BI-3802 for 16 hours and then sorted into _{eGFP}BCL6 reporter stable and unstable populations and for the resistance screen, SuDHL4_{Cas9} cells were treated with either 1 μM BI-3802 or DMSO for 20 days (n=4 guides/gene; two-sided empirical rank-sum test-statistics). FIG. 3D is a schematic and a bar graph. HEK293T_{Cas9} cells expressing the BCL6-BTB domain fused to the SIAH1 degron with indicated mutations (_{eGFP}BCL6¹⁻¹²⁹; linker; 241-260) in stability reporters were treated with DMSO, 1 μM BI-3802 (7 hours) or 1 μM BI-3802 (7 hours) and fluorescence monitored by flow cytometry. Bars represent mean±s.d. (n=3).

FIGS. 4A-4F are Western blots, a titration plot, and cell images showing that BCL6 polymerization enhances SIAH1 interaction and ubiquitination. FIG. 4A is a Western blot. Western blot analysis of eGFP immunoprecipitation in the presence of BI-3802 or DMSO from HEK293T_{Cas9} cells transduced with _{eGFP}BCL6 mutant/truncation constructs and _{V5}SIAH1^{44>S} (n=2, representative blot shown). FIG. 4B is a Western blot. Western blot analysis of _{Strep}BCL6⁵⁻³⁶⁰ in vitro ubiquitination by full length SIAH1 at varying time points in the presence of DMSO or 1 μM BI-3802 (n=2, representative blot shown). FIG. 4C is a titration plot. _{Bodipy}BCL6⁵⁻³⁶⁰ was titrated to 0.2 μM _{Biotin}SIAH1^{SBD} in presence of DMSO, 2 μM BI-3812, or 2 μM BI-3802, and the signal was measured by TR-FRET. Dots represent mean±s.d. Lines represent standard four parameter log-logistic curve fit (n=3). FIG. 4D is a bar plot. HEK293T cells transiently transfected with _{Nano-Luciferase}SIAH1^{C44S} and _{HaloTag}BCL6 constructs were treated with DMSO, 1 μM BI-3802 or 1 μM BI-3812 for 2 hours and the mBRET signal was measured. Bars represent mean±s.d. (n=3). ** p-value <0.01. *** p-value <0.001. One-sided t-test. FIG. 4E is a collection of cell images. HEK293T_{Cas9} cells expressing the _{eGFP}BCL6¹⁻²⁷⁵ stability reporter and _{V5}SIAH1 were pre-treated with 0.5 μM MLN7243 for 2 hours, then treated with 1 μM BI-3802 for 1 hour. The cells were fixed, permeabilized and stained with DAPI and anti-V5 antibodies and imaged by indirect immunofluorescence: BCL6 (FITC channel), SIAH1 (Alexa 633 channel), DNA (DAPI channel). FIG. 4F is a collection of cell images. HEK293T_{Cas9} cells expressing the _{eGFP}BCL6¹⁻²⁵⁰ stability reporter and _{V5}SIAH1 were treated with 0.5 μM MLN7243 for 2 hours and 1 μM BI-3802 for 1 hour. Fixation and staining were performed as described in FIG. 4E. Scale bars are 5 μm.

FIGS. 5A-5G are Western blots, bar graphs, plots, immunoblot images, and cell images showing characterization of BI-3802-induced BCL6 degradation. FIG. 5A is a Western blot. Western blot analysis of BCL6 levels in cytoplasmic, nuclear or chromatin bound fractions of SuDHL4 cells after 24 hours DMSO or 1 μM BI-3802 treatment (n=2, representative image shown). FIG. 5B is a bar graph. SuDHL4_{Cas9} cells were treated with 1 μM BI-3802 or DMSO for 1 hour and BCL6 mRNA levels were measured by quantitative PCR. Bars represent the mean±s.d. (n=3). FIG. 5C is a plot. SuDHL4_{Cas9} cells were exposed to 1 μM BI-3812 for 4 hours followed by whole proteome quantification using tandem mass tag mass spectrometry (mean fold change, p-value calculated by a moderated t-test, n=3). FIG. 5D is an image of an immunoblot. SuDHL4 cells were treated with 10 μM MG132 (26S proteasome inhibitor) for 1 hour, 1 μM BI-3802 for 45 minutes and 10 μM BI-3812 for 10 minutes. Protein lysates were run on a polyacrylamide gel and immunoblotted for the indicated targets. A subset of the polymerized BCL6 was insoluble and lost during the Western blot sample preparation, however, a treatment with an excess of BI-3812 shortly before protein harvest which reverts polymerization, solubilized BCL6 and allowed for reliable quantification. FIG. 5E is an image of an immunoblot. SuDHL4_{Cas9} cells were treated with DMSO, 10 μM MLN7243 (ubiquitin activating enzyme inhibitor), 10 μM MG132 (26S proteasome inhibitor), 10 μM Chloroquine (lysosomal inhibitor), or 5 μM MLN4924 (neddylation inhibitor) for 15 minutes, then, for indicated samples, 1 μM BI-3802 was added and 35 minutes later, 10 μM BI-3812 was added for the final 10 minutes, resulting in a total of 1 hour treatment with MLN7243, MG132, Chloroquine, and MLN4924, 45 minutes of BI-3802, and 10 minutes with BI-3812. Protein lysates were run on a polyacrylamide gel and immunoblotted for the indicated targets. FIG. 5F is a collection of cell images. Cytospin images of SuDHL4 cells treated with DMSO or 0.5 μM MLN7243 for 2 hours and 1 μM BI-3802 for 1 hour. The cells were fixed, permeabilized and stained with anti-BCL6 antibodies and imaged by indirect immunofluorescence: BCL6 (FITC channel). Scale bars are 5 μm. FIG. 5G is a plot. HEK293T cells expressing _{eGFP}BCL6¹⁻²⁷⁵ were exposed simultaneously to BI-3802 and BI-3812 for 24 hours and fluorescence monitored by flow cytometry. Points represent the mean±s.d. Lines represent standard four parameter log-logistic curve fit (n=3).

FIG. 6 presents computer-generated images showing computational docking of BCL6 helical filaments models with distinct binding modes. Visualization of top scoring BCL6-BTB domain filament model from three different binding modes: end-to-end (E2E), face-to-end (F2E) and face-to-face (F2F). Each BTB monomer used for building the tetramer model is labeled in a distinct shade of grey. BI-3802 is visualized as sphere. The interface score is an estimate of the binding energy between the dimers. The helical pitch was calculated by extending the tetramer. Sub-angstrom variations in the F2F binding mode has a profound effect on helical pitch (>10 nm).

FIGS. 7A-7E are schematics, plots, and a structural image presenting an analysis of BCL6-BTB variants in vivo. FIG. 7A is a schematic of the BCL6-BTB alanine variants resistance screen in SuDHL4 cells. FIG. 7B provides plots of read counts for selected BCL6-BTB alanine variants in the resistance screen were retrieved and their outgrowth visualized over the course of 21 days. FIG. 7C is a schematic of the BCL6-BTB alanine variants reporter screen in HEK293T cells. FIG. 7D is a plot. An alanine scan library, where each amino acid of full length BCL6 between positions 32 and 99 was individually mutated to alanine and each alanine to arginine. This library was introduced into the stability reporter, see FIG. 1C, and expressed in HEK293T cells. The mean±old-enrichment of read counts (_{eGFP}BCL6 mutant stable/_{eGFP}BCL6 mutant unstable) is shown on the linear BCL6 sequence and BCL6 alanine mutant, which impair 1 μM BI-3802 induced degradation (16 hours) are marked (over 3-fold enrichment, p-value <8e-05; n=4 codons/position; two-sided empirical rank-sum test-statistics). FIG. 7E is a plot showing correlation of BCL6 mRNA expression (TPM) and BCL6 dependency (CERES score) in a set of 559 cancer cell lines from the Dependency Map Project. Cell lines chosen for experiments are marked.

FIGS. 8A-8H are schematics, plots, and a bar graph presenting genome-scale CRISPR-Cas9 screens to identify the molecular machinery involved in BI-3802-induced degradation of BCL6. FIG. 8A is a schematic of the BCL6 stability reporter-based sorting screen. FIG. 8B is a plot showing median fold-enrichment of read counts (con BCL6 reporter stable/_{eGFP}BCL6 reporter unstable) and corresponding p-values for single guide RNAs (sgRNAs) targeting 19,112 human genes after 16 hours of treatment with 1 μM BI-3802 in HEK293T_{Cas9} cells (n=4 guides/gene; two-sided empirical rank-sum test-statistics). FIG. 8C is a plot showing median fold-enrichment of read counts (BCL6 stable/BCL6 unstable) and corresponding p-values for single guides RNA (sgRNAs) targeting 19,112 human genes after 16 hours of treatment with DMSO in HEK293T_{Cas9} cells (n=4 guides/gene; two-sided empirical rank-sum test-statistics). FIG. 8D is a plot showing normalized read counts for all sgRNAs in each sorted gate for non-targeting controls (NTC) and SIAH1. Symbols indicate the mean normalized read numbers for each sgRNA f s.d. (n=3). FIG. 8E is a bar graph. HEK293T_{Cas9} cells expressing the full length _{eGFP}BCL6 reporter were transfected with individual sgRNAs targeting the indicated genes and cultured for 6 days, then treated for 4 hours with DMSO or 1 μM BI-3802 and analyzed by flow cytometry. Symbols indicate results for different sgRNA guides. Bars represent mean±s.d. (n=3). FIG. 8F is a schematic of the genome-scale CRISPR-Cas9 resistance screen. FIG. 8G is a plot showing median fold-enrichment of read counts (BI-3802/DMSO treatment) and corresponding p-values for sgRNAs targeting 19,112 human genes after 20 days of treatment with either 1 μM BI-3802 or DMSO in SuDHL4_{Cas9} cells (n=4 guides/gene; two-sided empirical rank-sum test-statistics). FIG. 8H is a collection of plots. SuDHL4_{Cas9} cells were transduced with fluorescently labeled sgRNAs targeting SIAH1, grown for 5 days, and then exposed to DMSO or 1 μM BI-3802 for 20 days. The ratio of transduced to untransduced cells in the BI-3802 and DMSO arms was monitored over time using flow cytometry. Points represent mean±s.d. (n=3).

FIGS. 9A-9C are a bar graph, a sequence alignment, and a plot demonstrating that SIAH1 induces degradation of BCL6 via V×P motif. FIG. 9A is a bar graph. HEK293T_{Cas9} cells expressing the _{eGFP}BCL6 stability reporter were transfected with vectors expressing no-insert control, SIAH1, SIAH1C44S or SIAH2 and treated with DMSO or BI-3802 for 2 hours and fluorescence measured by flow cytometry. Bars represent the mean±s.d. (n=3). FIG. 9B is an alignment of amino acid sequences. Alignment of the BCL6 SIAH1 recognition site with previously published polypeptide sequences recognized by SIAH1 with inferred consensus SIAH1 binding site. FIG. 9C is a plot showing median fold-enrichment of read counts (BCL6 stable/BCL6 unstable) and corresponding p-values for single guides RNA (sgRNAs) targeting 713 E1, E2, E3, DUB and control genes (BISON library) after 16 hours of treatment with 1 μM BI-3802 in _{eGFP}BCL6^{1-129+linker+241-260} HEK293T_{Cas9} cells (n=4 guides/gene; two-sided empirical rank-sum test-statistics).

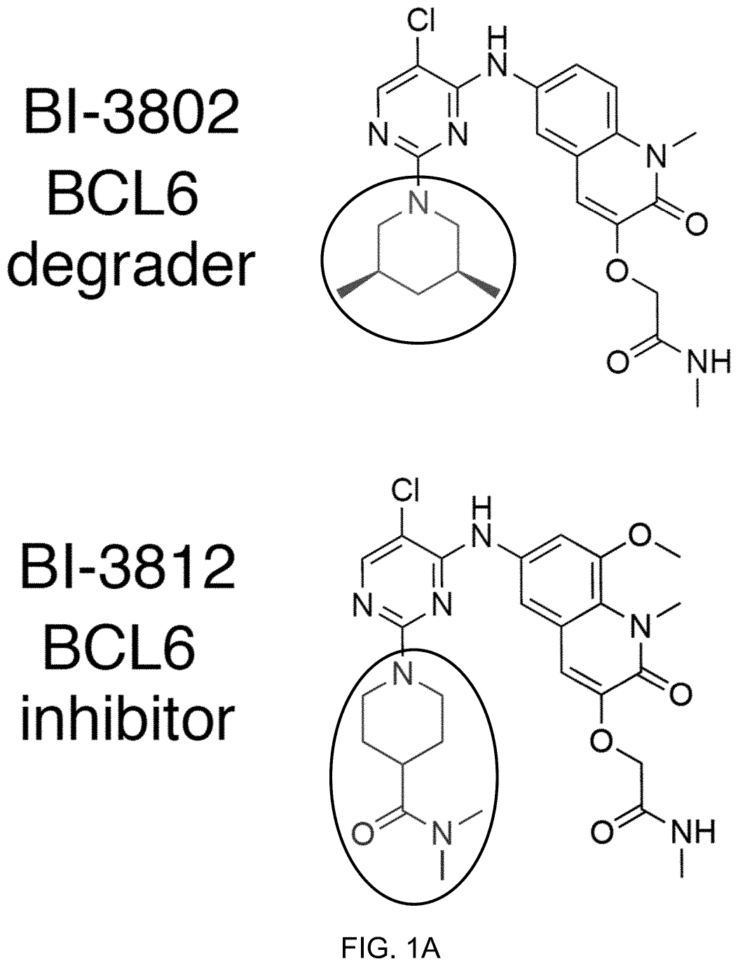

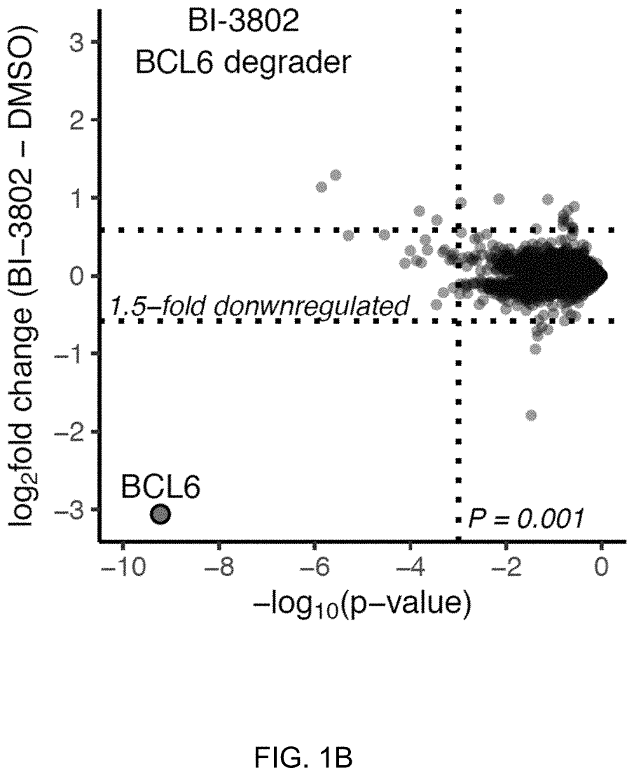

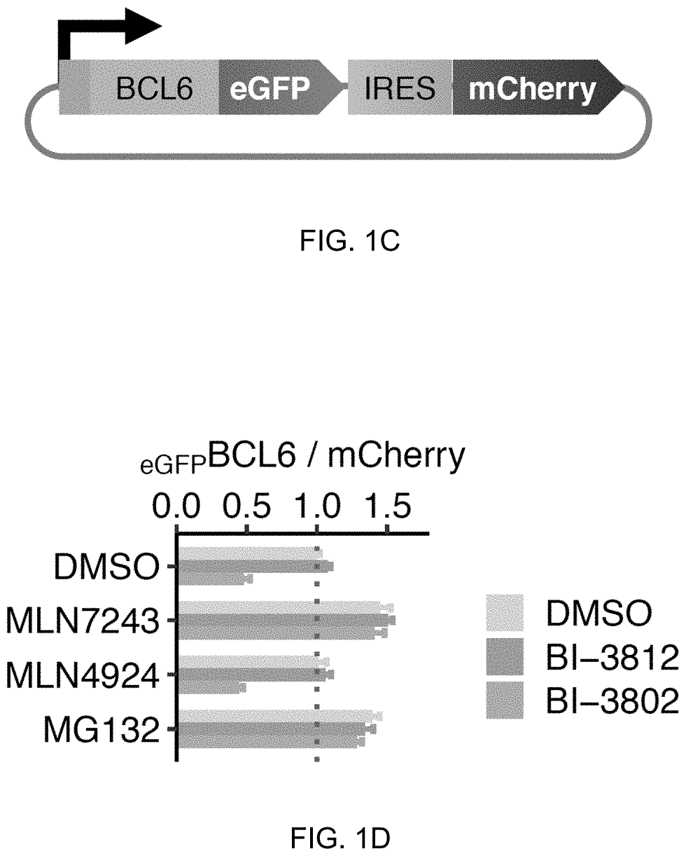

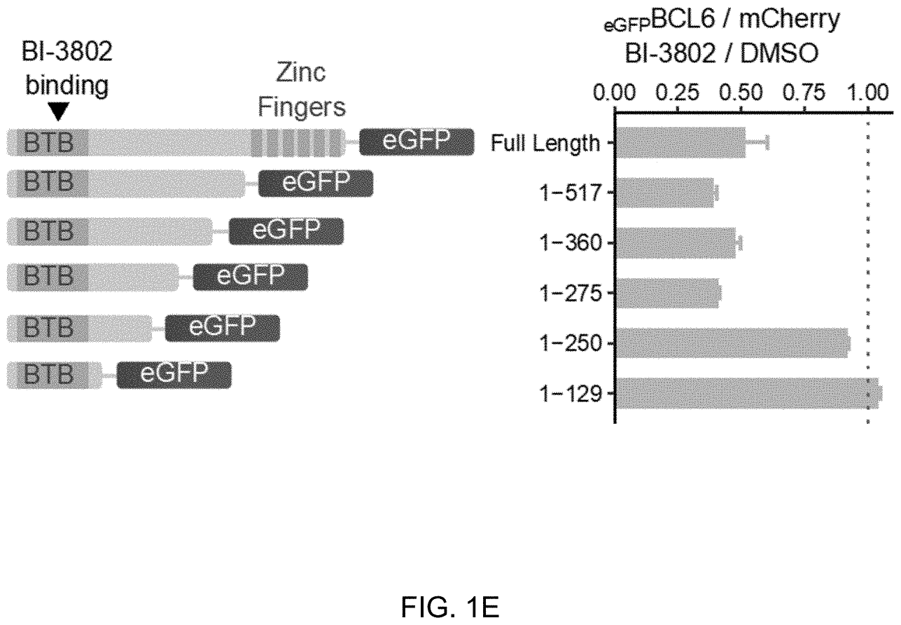

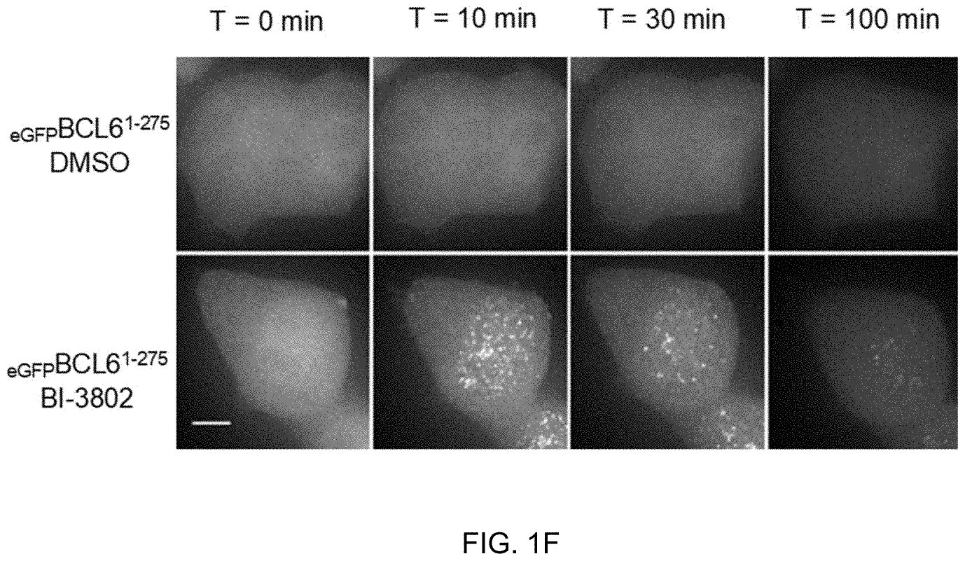

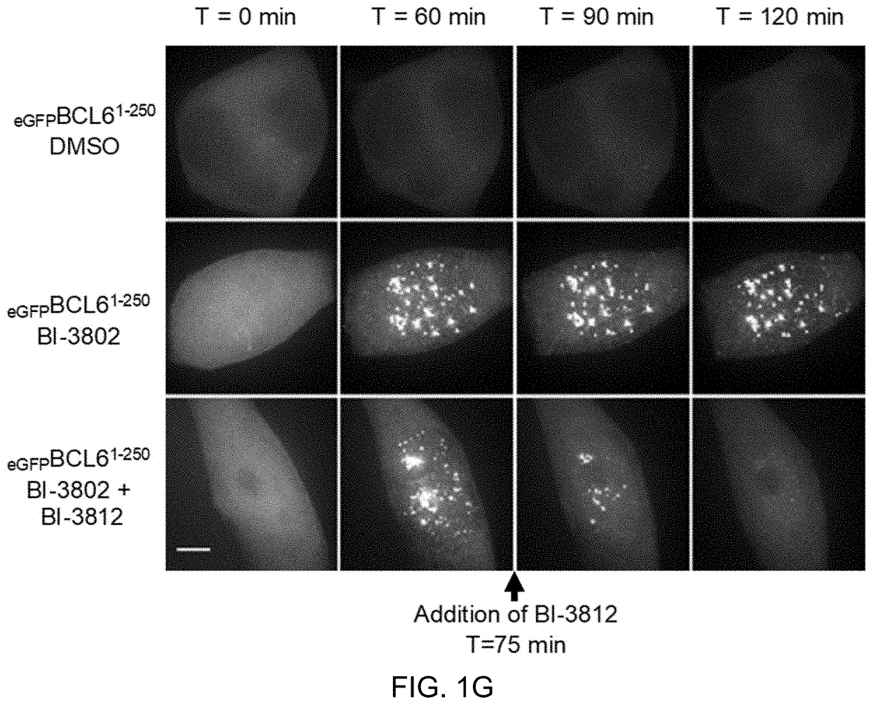

FIGS. 1A-10G are a gel image, plots, and Western blots showing characterization of SIAH1-mediated degradation of polymerized BCL6. FIG. 10A is an image of an SDS-page gel. SDS-page gel analysis of the in vitro pull-down between recombinant SIAH1^{SBD} and recombinant _{Strep}BCL6 in the presence of BI-3802 or DMSO. Strep, strep●Tag II (n=2, representative blot shown). FIG. 10B is a plot presenting a titration of BCL6²⁴¹⁻²⁶⁰ peptide binding to SIAH1 using isothermal calorimetry. FIG. 10C is a plot of a titration of SIAH1 binding to BCL6⁵⁻³⁶⁰ using isothermal calorimetry. FIG. 10D presents two Western blots. Recombinant _{Strep}BCL6⁵⁻³⁶⁰ was combined with full length SIAH1 and a panel of E2 enzymes (Boston Biochem) and screened for ubiquitination activity in vitro. Samples were run on a Western blot and visualized by strep●Tag II antibody-HRP conjugate. FIG. 10E is a plot. _{Bodipy}BCL6⁵⁻³⁶⁰ variants (WT, E41A, Y58A) were titrated to 0.2 μM _{Biotin}SIAH1^{SBD} in presence of 2 μM BI-3802, and the signal was measured by TR-FRET. Dots represent mean±s.d. Lines represent standard four parameter log-logistic curve fit (n=3). FIG. 10F is a plot. Preassembled 0.2 μM _{Bodipy}BCL6⁵⁻³⁶⁰ and 0.2 μM _{Biotin}SIAH1^{SBD} were exposed to increasing concentration of BI-3802 or BI-3812, and the signal was measured by TR-FRET. Dots represent mean±s.d. Lines represent standard four parameter log-logistic curve fit (n=3). FIG. 10G is a plot. Preassembled 0.1 μM _{FITC}BCoR polypeptide and 0.1 μM _{Biotin}BCL6⁵⁻¹²⁹ were exposed to increasing concentration of BI-3802 or BI-3812, and the signal measured by TR-FRET. Points represent mean±s.d. Lines represent standard four parameter log-logistic curve fit (n=3).

FIGS. 11A-11D are schematics and bar blots.

FIGS. 12A and 12B are cell images and a bar plot.

FIGS. 13A-13E provide cell images and plots. Top hits for BI-3802 E3 ligases+SIAH1 are marked on each plot.

### DETAILED DESCRIPTION OF THE INVENTION

The invention features compositions and methods that are useful in the development of therapeutics, synthetic biology, and characterizing resistance or sensitivity of a cancer or tumor cell to a compound.

The invention is based, at least in part, on the discovery that a compound induces highly specific, reversible polymerization, sequestration into cellular foci, and subsequent degradation of a target polypeptide. Not wishing to be bound to any mechanism of operation, the below Examples demonstrate that BI-3802 is a compound that binds the BTB domain of the oncogenic transcription factor BCL6 and such binding results in proteasomal degradation of BCL6. Drug-induced formation of BCL6 filaments can facilitate ubiquitination of BCL6 by the SIAH1 E3 ubiquitin ligase. These findings demonstrate that a compound can induce reversible polymerization coupled to highly specific polypeptide degradation, which in the case of BCL6 leads to superior pharmacological activity. These findings may create new avenues for the development of therapeutics and synthetic biology.

Not wishing to be bound to any theory of operation, through a combination of functional screens and biochemical dissection, the below Examples demonstrate that BI-3802 binding to the BCL6-BTB domain can trigger higher order assembly of BCL6 into filaments. Polymerization can promote ubiquitination of BCL6 by SIAH1, an E3 ligase that can recognize a V×P motif, and subsequent proteasomal degradation. BI-3802 can result in formation of intracellular foci containing BCL6 and SIAH1. The Examples provided below provide support for a mechanism by which a compound can inactivate a target through specific drug-induced polypeptide polymerization and subsequent degradation.

Not wishing to be bound by theory, structurally, BI-3802 and BI-3812, a compound that can induce polymerization of the BCL6 BTB domain and an inhibitor, respectively, differ in their solvent-exposed dimethyl-piperidine moiety. BI-3802 can induce polymerization of the BTB domain of BCL6 and foci formation in cells while BI-3812 may not. Since the solvent-exposed moiety in BI-3802 may trigger BCL6 polymerization, it is possible that modification of the solvent-exposed part in a compound (e.g., a small molecule compound) could induce new polypeptide-polypeptide interactions more generally. In the case of symmetric polypeptides, compounds (e.g., small molecule compounds) have the potential to induce polymerization which can then lead to degradation with specificity.

BI-3802, as well as structurally related bioavailable analogs (Bellenie, B. R. et al. Achieving In Vivo Target Depletion through the Discovery and Optimization of Benzimidazolone BCL6 Degraders. J Med Chem, doi:10.1021/acs.jmedchem.9b02076 (2020)), have markedly increased activity against lymphoma cells compared to BI-3812, which may be due to the combined effects of inhibiting co-activator binding, sequestering BCL6 into foci, and causing degradation of BCL6. Inhibition of BCL6 or degradation by proteolysis targeting chimeras (PROTACs) results in insufficient inhibition of downstream targets and consequently only minor anti-proliferative effects. Not wishing to be bound by theory, the unique mechanism of action of BI-3802 may overcome these limitations and help to explain its improved efficacy. The antiproliferative and transcriptional effect of BI-3802 is comparable to knock-out of BCL6 using an inducible CRISPR-Cas9 system. The molecular details provided in the below Examples enable optimization towards this mechanism of action, which could advance the development of therapeutics targeting malignancies driven by aberrant BCL6 activity.

BI-3802-induced BCL6 polymerization was competitively reversed by the BCL6 inhibitor (BI-3812). Thus, a non-degradable, isolated BCL6-BTB domain could be employed for synthetic biology applications as a reversible small-molecule induced polymerizer, a unique tool to trigger signaling events with precise temporal control. For example, in innate immunity and cell death pathways, signaling is often initiated by the formation of higher order structures, such as in the case of the signalosome or supramolecular organizing centers observed in Toll-like receptor or RIG1-like receptor signaling. At present, no tunable methods are available to control the formation and dissociation of these structures temporally for the recapitulation of nascent regulatory mechanisms in cells. Fusion of the BCL6-BTB domain to the target polypeptide as a polymerization module could provide a method to dissect the detailed downstream biology of these and other signaling pathways.

Drug-induced polymerization may expand the repertoire of pharmacologic modalities that mediate targeted polypeptide degradation as shown here for BCL6, with likely applications to other transcription factors and polypeptides with internal symmetry that have traditionally been difficult to drug. A subtle derivatization in solvent-exposed moiety distinguishes BI-3802 from BCL6 inhibitors that do not induce degradation, providing a potential path towards the rational design of molecules that induce polymerization.

### Recombinant Polypeptides

In some aspects, the invention provides a recombinant polypeptide (e.g., fusion polypeptide) comprising a Broad complex/Tamtrack/Brick-a-brack (BTB) domain.

In some embodiments, the recombinant polypeptide does not comprise a degron. In some embodiments, the polypeptide comprises an amino acid linker disposed at an N-terminus or at a C-terminus of the amino acid sequence derived from the BTB domain. The linker can have a length of 1, 5, 10, 15, 20, 30, 40, 50, or 100 amino acid residues. The linker can be disposed between the amino acid sequence derived from the Broad complex/Tamtrack/Brick-a-brack (BTB) domain and the degron.

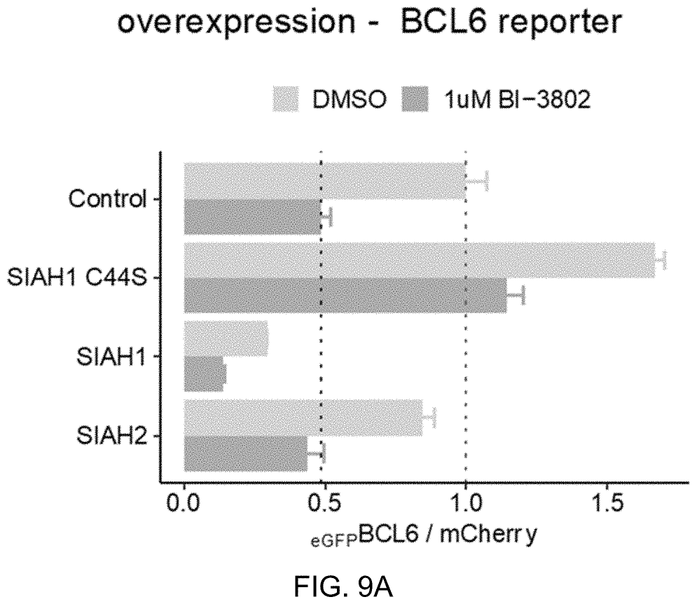

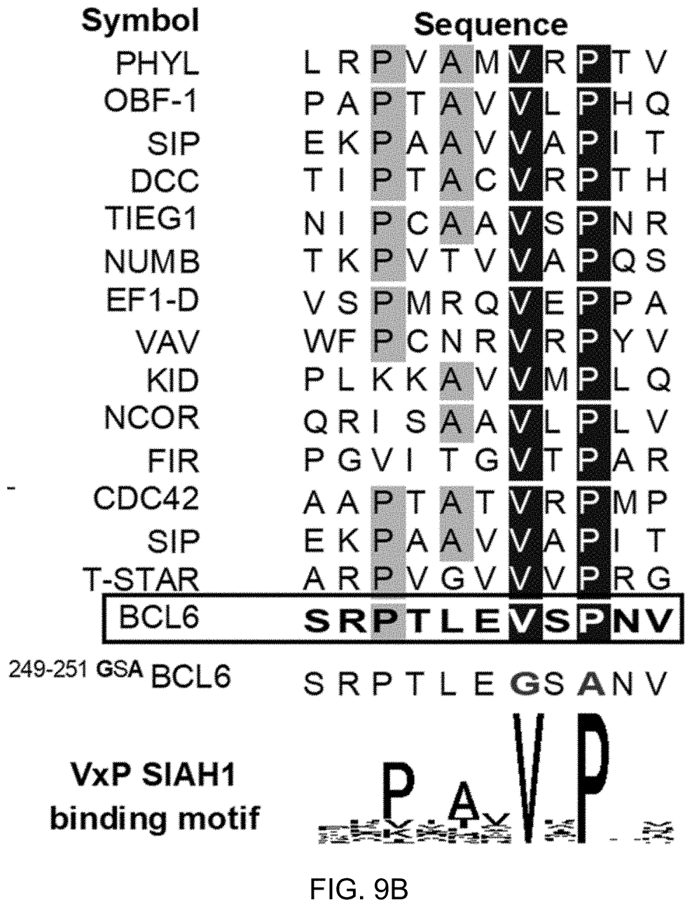

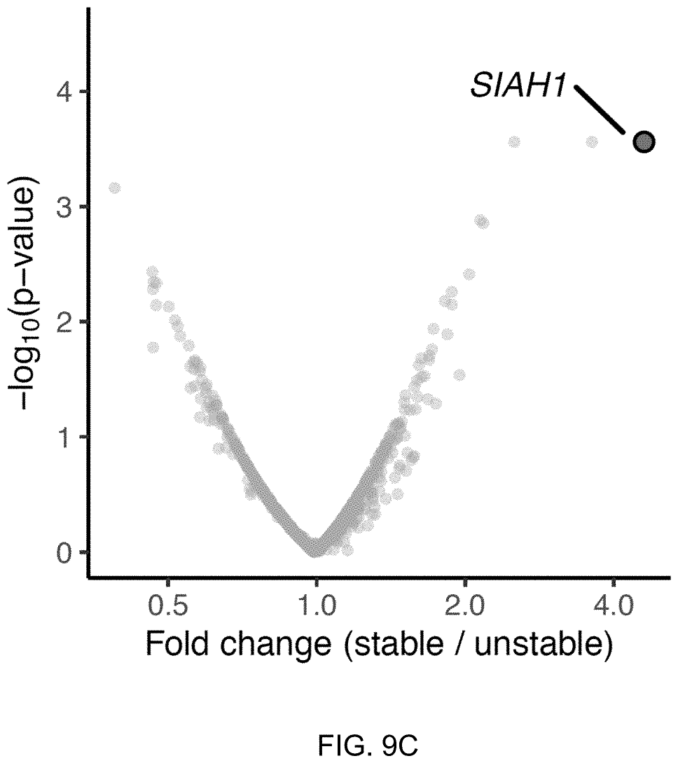

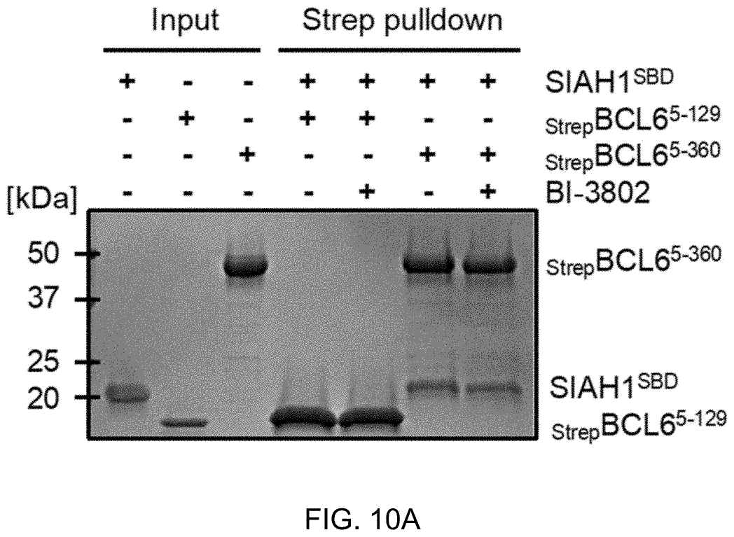

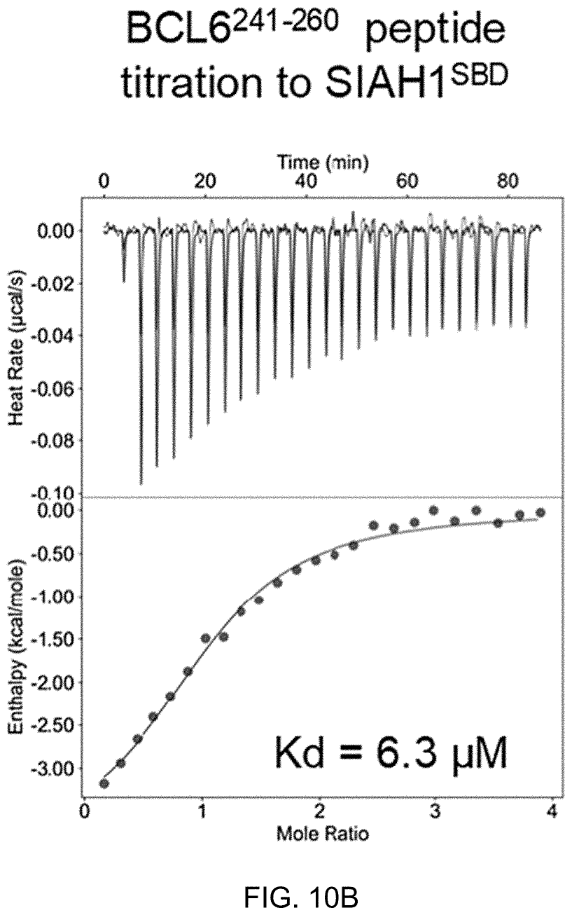

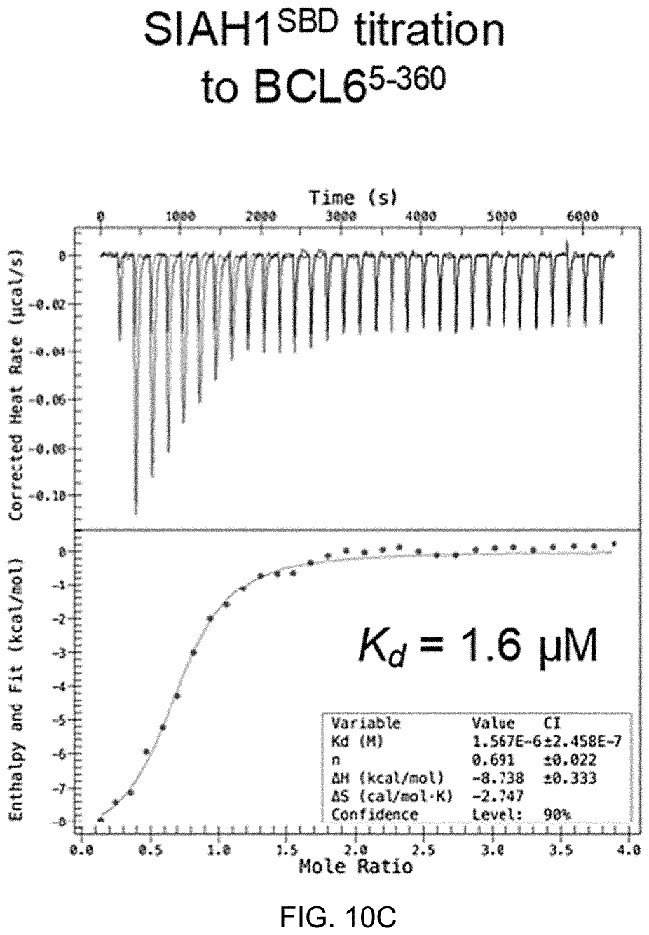

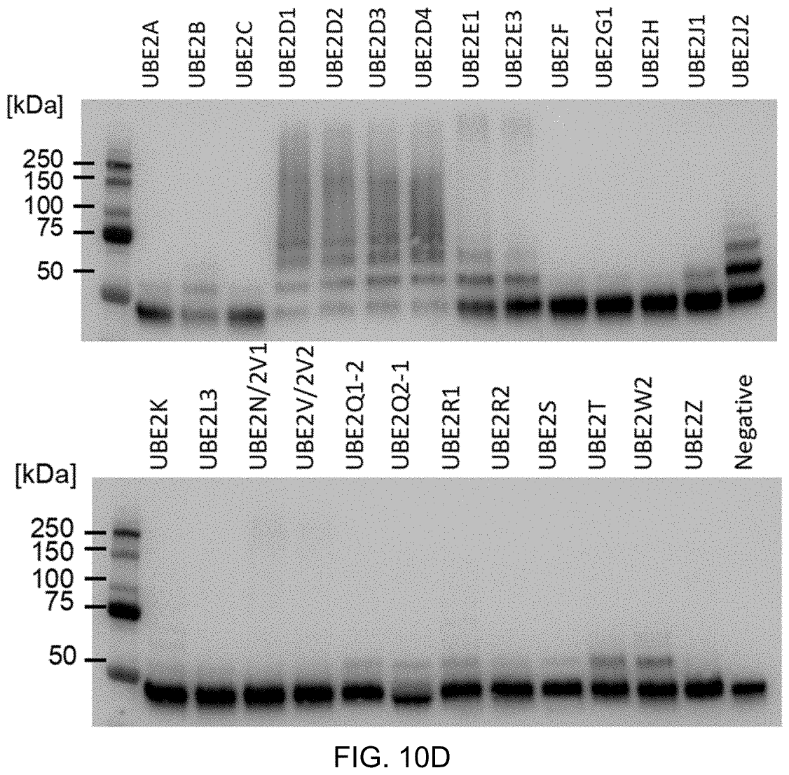

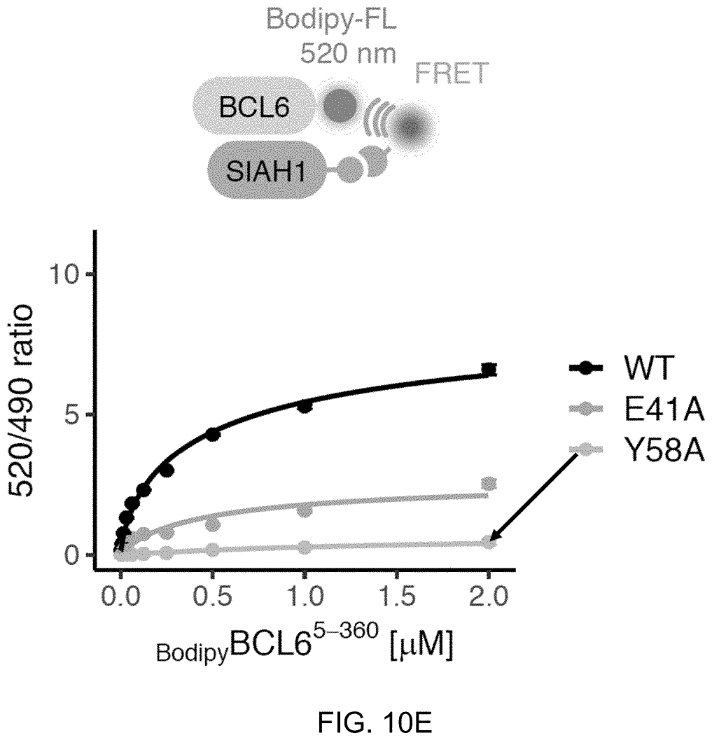

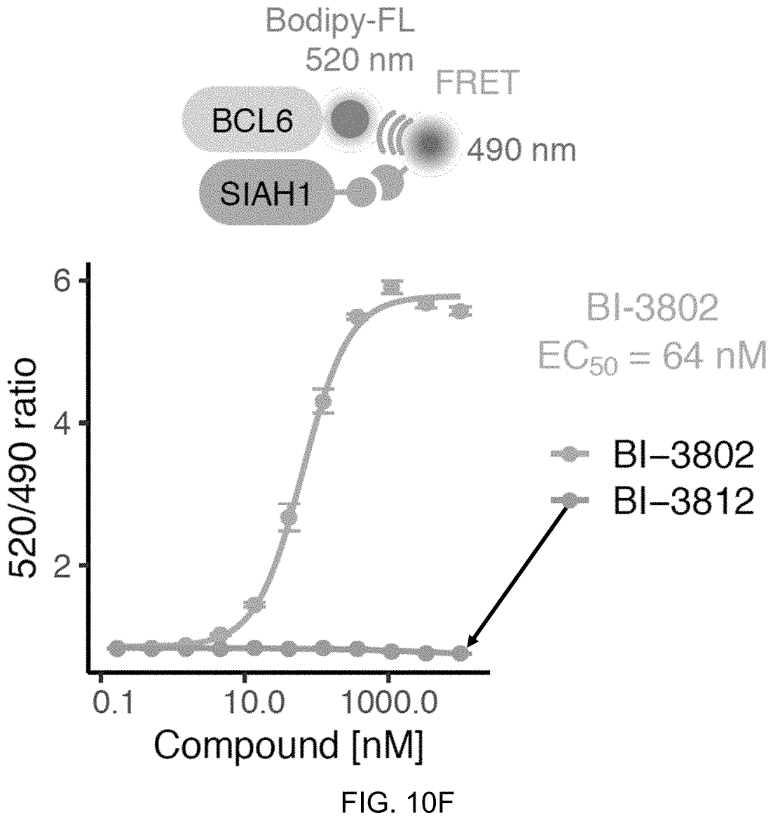

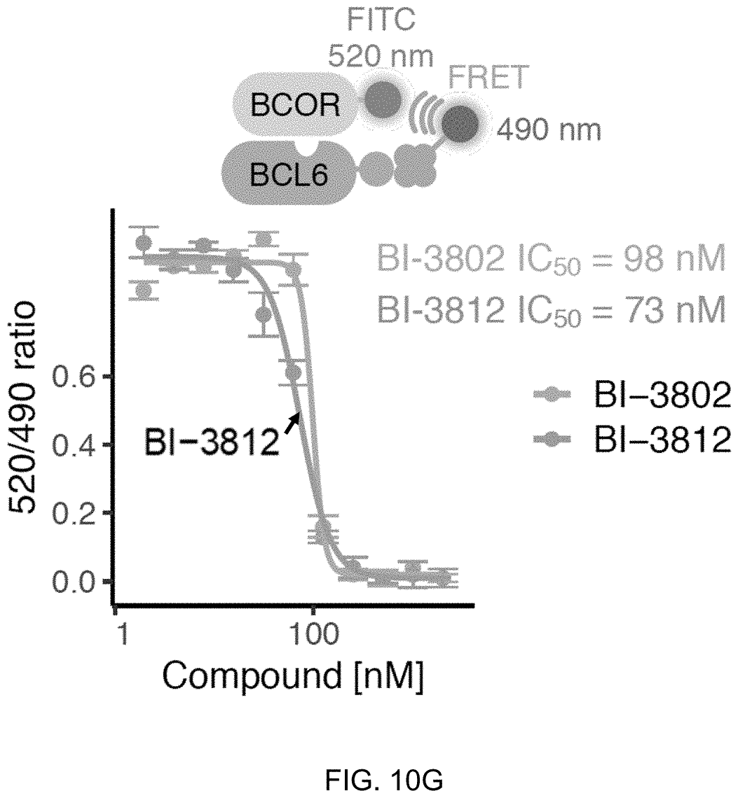

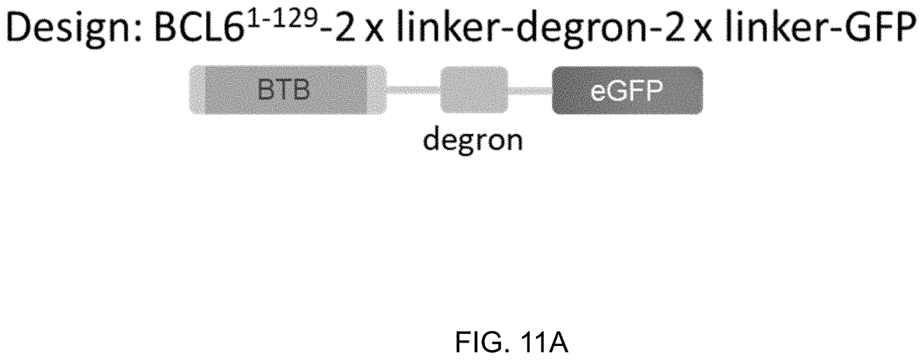

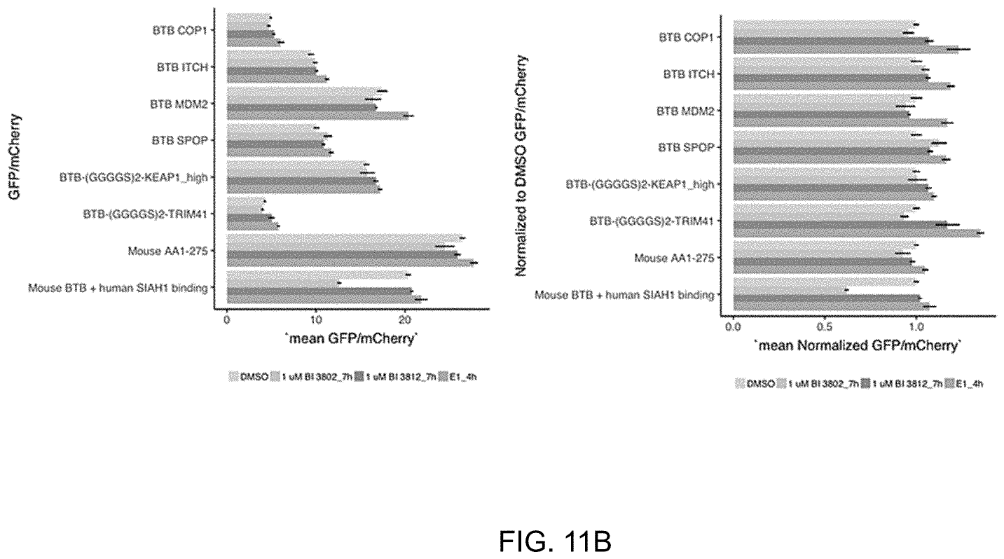

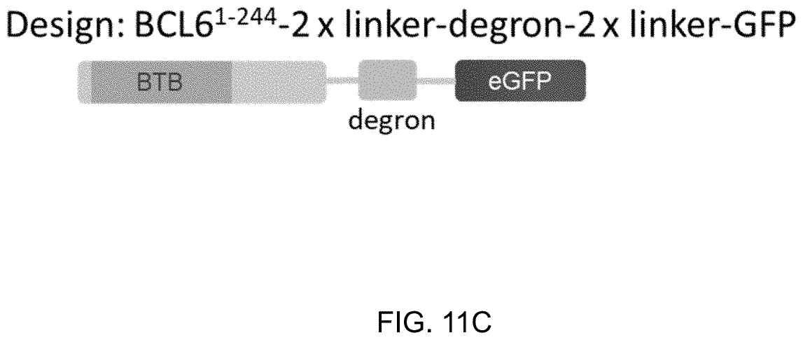

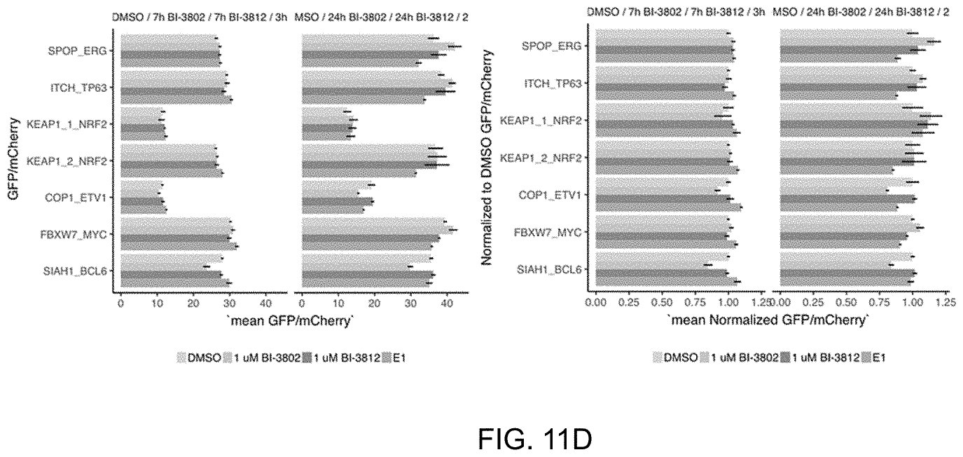

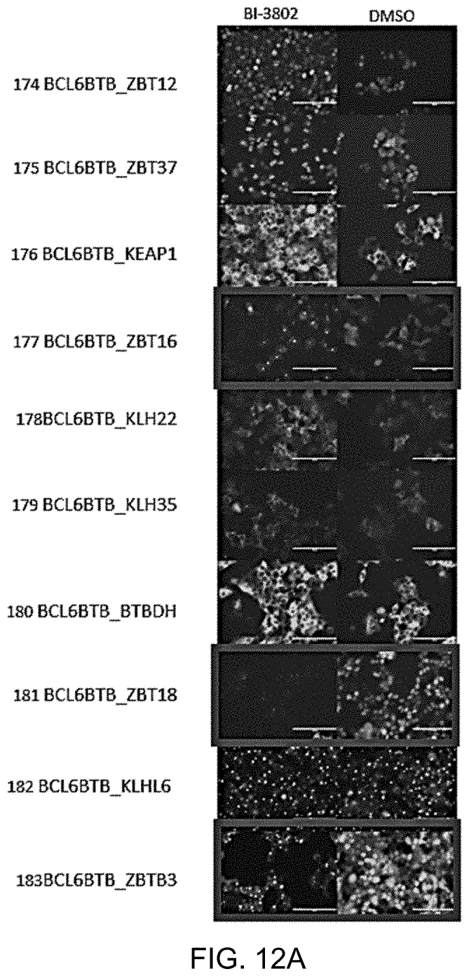

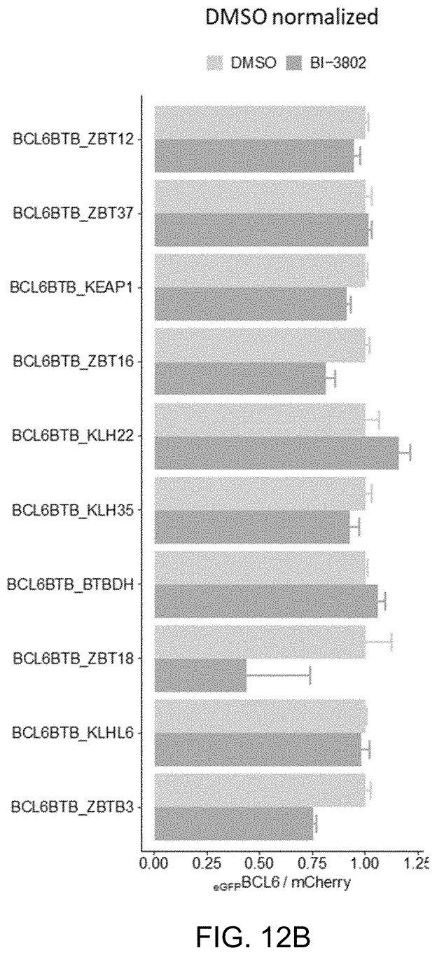

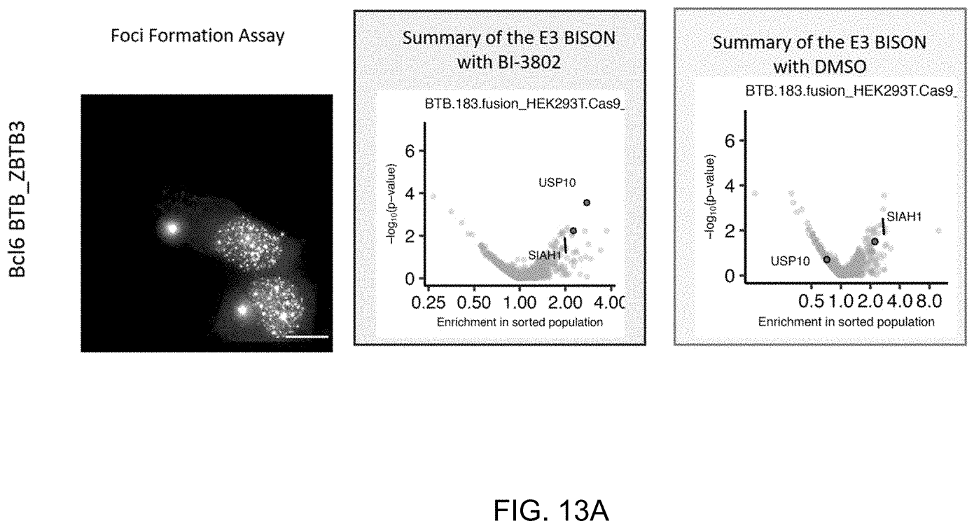

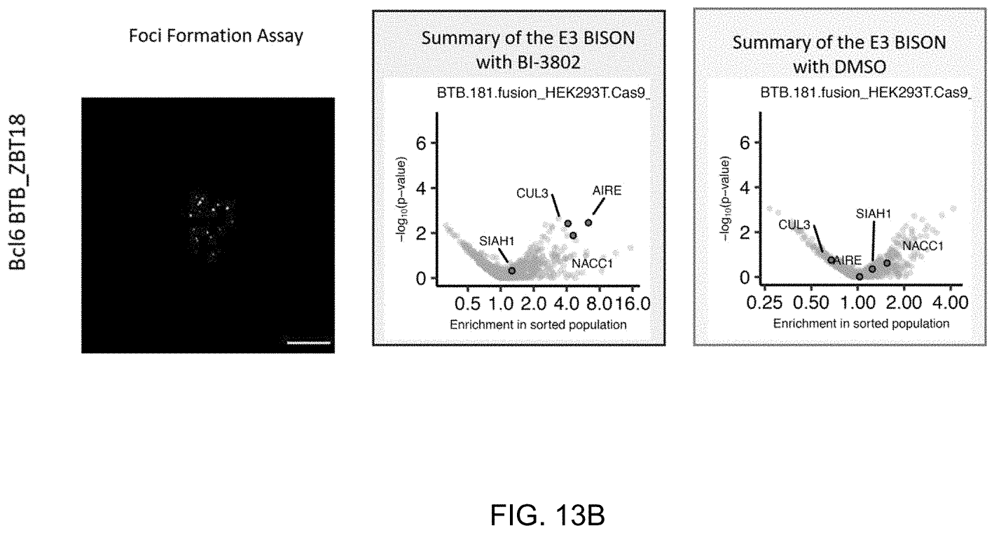

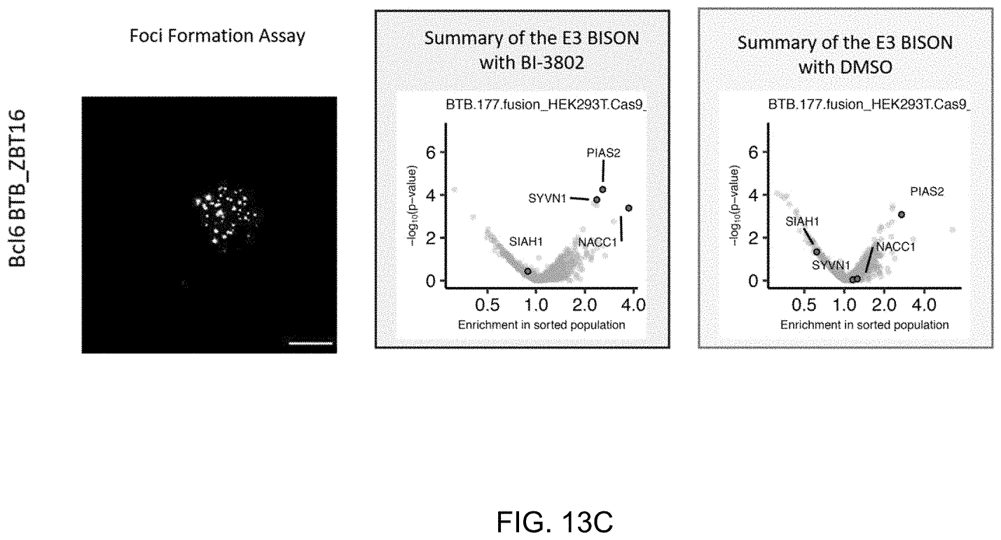

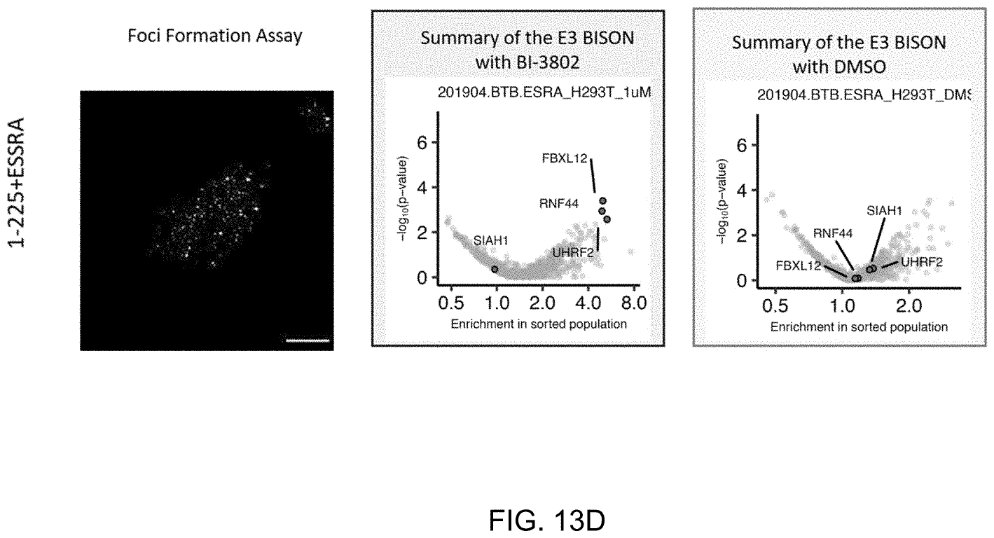

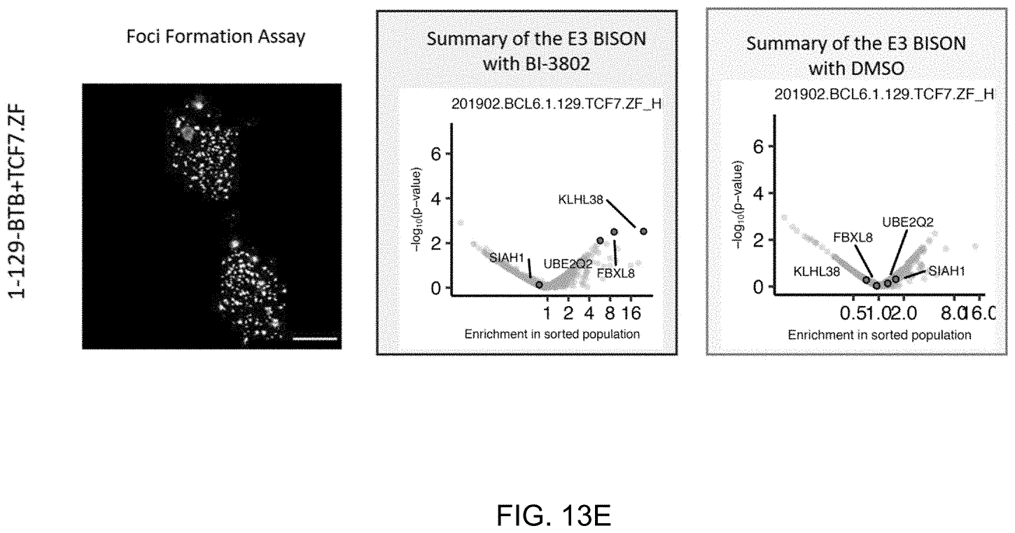

In some embodiments, the recombinant polypeptide comprises a degron. In some embodiments, the degron comprises the sequence V×P. In some embodiments, the degron comprises the conserved V×P E3 ubiquitin-protein ligase seven in absentia homolog 1 (SIAH1) binding motif depicted in FIG. 9B. In some embodiments, the degron comprises the sequence VSP. In some embodiments, the degron comprises any one of the individual V×P E3 ubiquitin-protein ligase seven in absentia homolog 1 (SIAH1) binding motifs listed in FIG. 9B. The degron can comprise amino acid residues 241-260 of BCL6.

In some embodiments, the polypeptide comprises a hydrophobic residue that mediates polymerization or oligomerization of the polypeptide upon compound binding, as described further below.

The recombinant polypeptide can comprise a fragment of BCL6. The fragment of BCL6 can comprise an amino acid sequence having about or at least about 60%, 65%, 70%, 75%, 80%, 85%, 90%, 95%, 96%, 97%, 98%, 99%, or 100% sequence identity to a BCL6 Broad complex/Tamtrack/Brick-a-brack (BTB) domain. The recombinant polypeptide can comprise an amino acid sequence derived from the first 100, 129, 150, 200, 250, 275, 300, 350, or 400 amino acid residues of BCL6.

In some embodiments the polypeptide comprises an amino acid sequence derived from the first 129 amino acid residues of BCL6. In some embodiments, the amino acid sequence derived from the BTB domain comprises an alteration at amino acid R28, E41, C84, G55, Y58, or a combination thereof, where the amino acid residue numbers refer to positions relative to the BCL6 sequence. In some embodiments, the amino acid sequence derived from the BTB domain does not comprise an alteration at one of, at any of, or at a combination of amino acids R28, E41, C84, G55, and Y58.

The recombinant polypeptide can be polymerizable, optionally reversibly polymerizable, as described further below. In some embodiments, polymerization of the recombinant polypeptide results in enhanced degradation of the polypeptide. Polymerization of the recombinant polypeptide can result in the formation of foci comprising the recombinant polypeptide. Degradation can be effected by E3 ubiquitin ligase. Polymerization may be reversed or inhibited by a BCL6 inhibitor, as described further below. Polymerization can be induced by a compound (e.g., a small molecule compound) as described further below. A representative compound is BI-3802, and analogs thereof, and a representative BCL6 inhibitor is BI-3812 and analogs thereof.

### Fusion Polypeptides

In some aspects, the invention provides an isolated fusion polypeptide comprising a BTB domain linked to a polypeptide of interest. The BTB domain can be linked at the C-terminus or at the N-terminus of the polypeptide of interest. The polypeptide of interest can be a heterologous amino acid sequence.

In some embodiments, the Broad complex/Tramtrack/Bric-a-brac (BTB) domain is disposed at or proximal to the C-terminus of the fusion polypeptide and the heterologous polypeptide is disposed at or proximal to an N-terminus of the fusion polypeptide. In some embodiments, the Broad complex/Tramtrack/Bric-a-brac (BTB) domain is disposed at or proximal to the N-terminus of the fusion polypeptide and the heterologous polypeptide is disposed at or proximal to a C-terminus of the fusion polypeptide. In some embodiments, the degron is disposed between the Broad complex/Tramtrack/Bric-a-brac (BTB) domain and the heterologous polypeptide. In some embodiments, the degron is disposed at a C-terminus or at an N-terminus of the fusion polypeptide. In some embodiments, the fusion polypeptide comprises a linker disposed between the Broad complex/Tramtrack/Bric-a-brac (BTB) domain and the degron, optionally wherein the linker is C-terminal or N-terminal to the BTB domain. In some embodiments, the fusion polypeptide has the architecture depicted in any one of FIG. 1E, 3D, 11A, or 11C.

In some embodiments, the fusion polypeptide comprises a detectable moiety, such as a fluorescent amino acid sequence. Non-limiting examples of fluorescent polypeptides include green fluorescent protein (GFP or enhanced GFP), red fluorescent protein (RFP), mCherry, etc.

In some embodiments, the polypeptide of interest is a transcription factor, is a marker of disease, is an enzyme, is a signaling polypeptide, or is an oncogene. In some embodiments, the polypeptide of interest is a fragment of a transcription factor, is a fragment of an oncogene, is a fragment of an enzyme, is a fragment of a signaling polypeptide, or is a fragment of a marker of disease. The transcription factor can have DNA binding activity. The signaling polypeptide can have signal transduction activity.

In some embodiments, the polypeptide of interest is ZBT12, ZBT37, KEAP1, ZBT16, KLH22, KLH35, BTBDH, ZBT18, KLHL6, or ZBTB3. In some embodiments, the heterologous polypeptide is derived from a mammalian polypeptide, optionally a mouse or a human. In some embodiments, the heterologous polypeptide is derived from a yeast, a unicellular organism, a prokaryote, a plant, a vertebrate, or an invertebrate. The heterologous polypeptide can be an enzyme or a signaling polypeptide.

In various embodiments, the fusion polypeptide undergoes polymerization when contacted with an agent. In some embodiments, polymerized fusion polypeptides sequester into cellular foci. In some embodiments, polymerization of the fusion polypeptides results in enhanced degradation of the fusion polypeptides.

### Recombinant Polypeptide Expression

In some aspects, the invention provides a method for expressing a recombinant polypeptide (e.g., a fusion polypeptide). In some embodiments, the method comprises delivering a nucleotide molecule encoding the fusion polypeptide to a cell. In various embodiments, the cell is a disease cell (e.g., a cancer or tumor cell). In some embodiments, the cell is a eukaryotic cell, a yeast cell, an insect cell, a prokaryotic cell, a bacterial cell (e.g., E. coli), or a mammalian cell. The eukaryotic cell can be an HEK293 cell, a CHO-S cell, a COS7 cell, a CHO cell, a 293T cell, a Hela cell, a HEK293 cell, a CHO-K1 cell, a SuDHL4 cell, a Raji cell, a DEL cell, a HEK293T cell, a T cell, a B cell, a Vero cell, or a DG44 cell. In various embodiments, the cell expresses Cas9. The cell can be an insect cell; as non-limiting examples, the cell can be a Spodoptera frugiperda (Sf9) cell or a trichoplusiani High Five insect cell. In some embodiments, the fusion polypeptide is expressed using a baculovirus expression system. In some embodiments, the fusion polypeptide may be expressed in vitro outside of a cell. The recombinant polypeptide may be constitutively expressed or its expression may be regulated by an inducible promoter or other control mechanism where conditions necessitate highly controlled regulation or timing of the expression of a polypeptide, enzyme, or other cell product. In some embodiments, the cell is a yeast cell; as non-limiting examples, the cell can be a Saccharomyces cerevisiae, a Pichia pastoris, a Hansenula polymorpha, a Yarrowia lipolvtica, an Arxula adeninivorans, a Kluyveromyces lactis, a Candida boidinii or a Schizosaccharomyces pombe yeast cell. The cell can be a bacterial (e.g., Escherichia coli) or an archaeal cell.

In one aspect, the invention provides a cell comprising a nucleotide molecule encoding the recombinant polypeptide. In one aspect, the invention provides a cell comprising the recombinant polypeptide. The cell can be an isolated cell and any one of the cell types described above.

The cell of the invention, its progenitor or its in vitro-derived progeny, can contain a heterologous nucleotide sequence encoding genes to be expressed. Insertion of one or more pre-selected nucleotide molecules can be accomplished by homologous recombination or by viral integration into the host cell genome. The desired nucleotide molecule can also be incorporated into the cell, particularly into its nucleus, using a plasmid expression vector and a nuclear localization sequence. Methods for directing nucleotide molecules to the nucleus have been described in the art. The nucleotide molecules can be introduced using promoters that will allow for the gene of interest to be positively or negatively induced using certain chemicals/drugs, to be eliminated following administration of a given drug/chemical, or can be tagged to allow induction by chemicals, or expression in specific cell compartments.

Calcium phosphate transfection can be used to introduce plasmid DNA containing a target gene or polynucleotide into a cell and is a standard method of DNA transfer to those of skill in the art. DEAE-dextran transfection, which is also known to those of skill in the art, may be preferred over calcium phosphate transfection where transient transfection is desired, as it is often more efficient. Since the cells of the present invention can be isolated cells, microinjection can be particularly effective for transferring genetic material into the cells. This method is advantageous because it provides delivery of the desired genetic material directly to the nucleus, avoiding both cytoplasmic and lysosomal degradation of the injected polynucleotide. Cells of the present invention can also be genetically modified using electroporation.

Liposomal delivery of nucleotide molecules to genetically modify the cells can be performed using cationic liposomes, which form a stable complex with the polynucleotide. For stabilization of the liposome complex, dioleoyl phosphatidylethanolamine (DOPE) or dioleoyl phosphatidylcholine (DOPQ) can be added. Commercially available reagents for liposomal transfer include Lipofectin (Life Technologies). Lipofectin, for example, is a mixture of the cationic lipid N-[1-(2,3-dioleyloxy)propyl]-N—N—N-trimethyl ammonia chloride and DOPE. Liposomes can carry nucleotide molecules, can generally protect the polynucleotide from degradation, and can be targeted to specific cells or tissues. Cationic lipid-mediated gene transfer efficiency can be enhanced by incorporating purified viral or cellular envelope components, such as the purified G glycoprotein of the vesicular stomatitis virus envelope (VSV-G). Gene transfer techniques which have been shown effective for delivery of nucleotide molecules into primary and established mammalian cell lines using lipopolyamine-coated nucleotide molecules can be used to introduce target DNA into the lymphatic endothelial progenitor cells described herein.

Naked plasmid DNA can be injected directly into a tissue comprising cells of the invention. This technique has been shown to be effective in transferring plasmid DNA to skeletal muscle tissue, where expression in mouse skeletal muscle has been observed for more than 19 months following a single intramuscular injection. More rapidly dividing cells take up naked plasmid DNA more efficiently. Therefore, it is advantageous to stimulate cell division prior to treatment with plasmid DNA. Microprojectile gene transfer can also be used to transfer nucleotide molecules into cells either in vitro or in vivo. The basic procedure for microprojectile gene transfer was described by J. Wolff in Gene Therapeutics (1994), page 195. Similarly, microparticle injection techniques have been described previously, and methods are known to those of skill in the art. Signal peptides can be also attached to plasmid DNA to direct the DNA to the nucleus for more efficient expression.

Viral vectors are used to genetically alter cells of the present invention and their progeny. Viral vectors are used, as are the physical methods previously described, to deliver one or more target genes, polynucleotides, antisense molecules, or ribozyme sequences, for example, into the cells. Viral vectors and methods for using them to deliver DNA to cells are well known to those of skill in the art. Examples of viral vectors that can be used to genetically alter the cells of the present invention include, but are not limited to, adenoviral vectors, adeno-associated viral vectors, retroviral vectors (including lentiviral vectors), alphaviral vectors (e. g., Sindbis vectors), and herpes virus vectors.

Peptide or polypeptide transfection is another method that can be used to genetically alter lymphatic endothelial progenitor cells of the invention and their progeny. Peptides such as Pep-1 (commercially available as Chariot), as well as other polypeptide transduction domains, can quickly and efficiently transport biologically active polypeptides, peptides, antibodies, and nucleic acids directly into cells, with an efficiency of about 60% to about 95% (Morris, M. C. et al, (2001) Nat. Biotech. 19: 1173-1176).

The method can further include integrating the nucleotide molecule encoding the fusion polypeptide into a genome of a cell, optionally using any of various techniques known in the art including use of a clustered regularly interspaced short palindromic repeats (CRISPR) system.

Any of various techniques known in the art can be used to introduce the fusion polypeptide or nucleotide molecule into a cell, including those described below; e.g., electrolysis, transfection, or transformation. In some embodiments, the fusion polypeptide or nucleotide molecule is introduced to a cell using a virus, optionally a lentivirus.

### Nucleotide Constructs

In some aspects, the invention provides a polynucleotide molecule encoding a recombinant polypeptide described herein. In some embodiments, the polynucleotide molecule comprises DNA and/or RNA. In some embodiments, the fusion polypeptide is encoded by a polynucleotide molecule present in a plasmid or vector. In some embodiments, the vector is a plasmid (e.g., Cilantro2, Artichoke, or a pAC-derived vector) and/or virus (e.g., a Lentivirus). The recombinant nucleotide molecule can be linear or covalently-closed-circular. In some embodiments, the vector is a stability vector, a non-limiting example of which is shown in FIG. 1C. The recombinant polynucleotide molecule can be an expression vector. The polynucleotide molecule can comprise a promoter sequence.

The recombinant polynucleotide can be a mammalian expression vector. The expression vector can be a viral vector including, as non-limiting examples, a lentiviral, baculoviral, adenoviral, or adeno-associated virus vector. In some embodiments, viral vectors are used to genetically alter cells of the present invention and their progeny. Viral vectors are used, as are the physical methods previously described, to deliver one or more target genes, polynucleotides, antisense molecules, or ribozyme sequences, for example, into the cells. Viral vectors and methods for using them to deliver DNA to cells are well known to those of skill in the art. Examples of viral vectors that can be used to genetically alter the cells of the present invention include, but are not limited to, adenoviral vectors, adeno-associated viral vectors, retroviral vectors (including lentiviral vectors), alphaviral vectors (e. g., Sindbis vectors), and herpes virus vectors.

### Methods for Inducing Polymerization

In some aspects, the invention provides a method for inducing polymerization of a polypeptide of interest or a plurality of distinct polypeptides of interest. In some aspects, the invention provides a method for altering activity of a polypeptide of interest or a plurality of distinct polypeptides of interest. The polypeptide or plurality of distinct polypeptides may comprise an enzyme. In some aspects, the invention provides a method for enhancing degradation of a polypeptide, optionally proteasomal degradation. In some aspects, the invention provides a method for inducing co-localization of a plurality of polypeptides. The plurality of polypeptides may comprise the same or distinct polypeptides. The method can comprise operably linking a polypeptide of interest to a BTB domain to generate a fusion polypeptide. The method can be carried out in vitro or in a cell.

In various embodiments, the polypeptide is the fusion polypeptide described above. In some embodiments, the method includes contacting the polypeptide with a compound (e.g., a small molecule compound), that induces polymerization of the polypeptide. In some embodiments, the method comprises contacting a cell expressing the polypeptide with the compound.

In some embodiments, polymerization of the polypeptide enhances degradation or alters or enhances an activity of the polypeptide. Polymerization of the polypeptide can localize the fusion polypeptide within the cell, optionally within foci. In some embodiments, the localization increases the concentration of the polypeptide within a portion of the cell. The degradation can be mediated by the proteasome. Polymerization can enhance the rate of ubiquitination of the polypeptide. Degradation of the polypeptide can reduce intracellular activity or intracellular concentrations of the polypeptide in a cell. In some embodiments polymerization of the polypeptide enhances ubiquitination of the polypeptide, optionally by an E3 ligase (e.g., seven in absentia homolog 1 (SIAH1) or seven in absentia homology 2 (SIAH2)). Polymerization of the polypeptide can result in the formation of foci within a cell expressing the polypeptide.

In some embodiments, the method includes introducing a Broad complex/Tramtrack/Bric-a-brac (BTB) domain into the genome of a cell such that the cell expresses a fusion polypeptide comprising the BTB domain linked to a polypeptide endemic to the genome of the cell.

In various embodiments, the Broad complex/Tramtrack/Bric-a-brac (BTB) domain comprises mutations that reduce or prevent degradation and/or polymerization of the BTB domain, optionally following polymerization. In some embodiments, the Broad complex/Tramtrack/Bric-a-brac (BTB) domain comprises a R28A, E41A, C84A, G55A, Y58A mutation or a combination thereof. In some embodiments, the Broad complex/Tramtrack/Bric-a-brac (BTB) domain and/or the fusion polypeptide or polypeptide does not comprise a V×P motif. In some embodiments, the Broad complex/Tramtrack/Bric-a-brac (BTB) domain, and/or the fusion polypeptide or polypeptide does not comprise a degron.

In various embodiments, the method for enhancing degradation of a polypeptide includes contacting a fusion polypeptide with the compound, wherein the fusion polypeptide comprises a Broad complex/Tramtrack/Bric-a-brac (BTB) that does not comprise a R28A, E41A, C84A, G55A, Y58A mutation or does not comprise a combination thereof. In some embodiments of the method, the fusion polypeptide comprises a degron, optionally wherein the degron is a V×P motif.

In various embodiments, polymerization of the polypeptide results in enhancing an activity of the polypeptide, optionally wherein the activity is an enzymatic activity.

In some embodiments, the method includes causing a plurality of unique or identical fusion polypeptides to polymerize with one another. In some embodiments, the method includes fusing Broad complex/Tramtrack/Bric-a-brac (BTB) domains to a plurality of unique or identical heterologous polypeptides to provide a plurality of unique or identical fusion polypeptides. In some embodiments the Broad complex/Tramtrack/Bric-a-brac (BTB) domains all comprise the same amino acid sequence derived from the same BTB domain, or dissimilar amino acid sequences derived from different BTB domains. Contacting the plurality of fusion polypeptides with the compound can induce polymerization of the fusion polypeptides with one another and can increase enzymatic, signaling, or metabolic interactions between the fusion polypeptides.

In various embodiments, the polymerization is reversible. In some embodiments, the polymerization can be reversed or inhibited by contacting the fusion polypeptides with an inhibitor.

In some embodiments the compound and/or inhibitor can be a quinolinone compound or a benzimidazolone compound.

The compound can be BI-3802 (CAS No.: 2166387-65-9; 2-((6-((5-chloro-2-((3S,5R)-3,5-dimethylpiperidin-1-yl)pyrimidin-4-yl)amino)-1-methyl-2-oxo-1,2-dihydroquinolin-3-yl)oxy)-N-methylacetamide) or an analog of BI-3802. In some embodiments, the compound is selected from the molecules disclosed in Bellenie, et al. “Achieving in vivo target depletion through the discovery and optimization of benzimidaxolone BCL6 degraders,” Journal of Medicinal Chemistry, 63:4047-4068 (2020). In some embodiments the compound is 5-((5-Chloro-2-(3-methylpiperidin-1-yl)pyrimidin-4-yl)-amino)-3-(3-hydroxy-3-methylbutyl)-1-methyl-1,3-dihydro-2H-benzo[d]imidazol-2-one; 5-((5-Chloro-2-((3S,5R)-3,5-dimethylpiperidin-1-yl)-pyrimidin-4-yl)amino)-3-(3-hydroxy-3-methylbutyl)-1-methyl-1,3-dihydro-2H-benzo[d]imidazol-2-one; 5-((5-Chloro-2-((3S,5R)-3,5-dimethylpiperidin-1-yl)pyridin-4-yl)amino)-3-(3-hydroxy-3-methylbutyl)-1-methyl-1,3-dihydro-2H-benzo[d]imidazol-2-one; 5-((5-Chloro-2-(3-(trifluoromethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(4,4-difluoropiperidin-1-yl)pyrimidin-4-yl)-amino)-3-(3-hydroxy-3-methylbutyl)-1-methyl-1,3-dihydro-2H-benzo[d]imidazol-2-one; 5-((5-Chloro-2-(4,4-difluoro-3-methylpiperidin-1-yl)-pyrimidin-4-yl)amino)-3-(3-hydroxy-3-methylbutyl)-1-methyl-1,3-dihydro-2H-benzo[d]imidazol-2-one; 5-((5-Chloro-2-((3R,5S)-4,4-difluoro-3,5-dimethylpiperidin-1-yl)pyrimidin-4-yl)amino)-3-(3-hydroxy-3-methylbutyl)-1-methyl-1,3-dihydro-2H-benzo-[d]imidazol-2-one, 5-((5-Chloro-2-(3-(hydroxymethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(4,4-difluoro-3-(hydroxymethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(4,4-difluoro-3-(methoxymethyl)piperidin-1-yl)pyrimidin-4-yl)amino)-3-(3-hydroxy-3-methylbutyl)-1-methyl-1,3-dihydro-2H-benzo[d]imidazol-2-one; or various combinations thereof. In some embodiments, the compound is 5-((5-Chloro-2-(dimethylamino)pyrimidin-4-yl)amino)-3-(3-hydroxy-3-methylbutyl)-1-methyl-1,3-dihydro-2H-benzo[d]-imidazol-2-one; 5-((5-Chloro-2-morpholinopyrimidin-4-yl)amino)-3-(3-hydroxy-3-methylbutyl)-1-methyl-1,3-dihydro-2H-benzo[d]-imidazol-2-one; 5-((5-Chloro-2-(piperidin-1-yl)pyrimidin-4-yl)amino)-3-(3-hydroxy-3-methylbutyl)-1-methyl-1,3-dihydro-2H-benzo[d]-imidazol-2-one; 5-((5-Chloro-2-((2R,6S)-2,6-dimethylmorpholino)pyridin-4-yl)amino)-3-(3-hydroxy-3-methylbutyl)-1-methyl-1,3-dihydro-2H-benzo[d]imidazol-2-one; 2-Chloro-4-((3-(3-hydroxy-3-methylbutyl)-1-methyl-2-oxo-2,3-dihydro-1H-benzo[d]imidazol-5-yl)amino)nicotinonitrile; 5-((2,3-Dichloropyridin-4-yl)amino)-3-(3-hydroxy-3-methylbutyl)-1-methyl-1,3-dihydro-2H-benzo[d]imidazol-2-one; 5-((3-Chloropyridin-4-yl)amino)-3-(3-hydroxy-3-methylbutyl)-1-methyl-1,3-dihydro-2H-benzo[d]imidazol-2-one; 4-Chloro-6-((3-(3-hydroxy-3-methylbutyl)-1-methyl-2-oxo-2,3-dihydro-1H-benzo[d]imidazol-5-yl)amino)pyrimidine-5-Carbonitrile; 5-((5,6-Dichloropyrimidin-4-yl)amino)-3-(3-hydroxy-3-methylbutyl)-1-methyl-1,3-dihydro-2H-benzo[d]imidazol-2-one; 5-((3,5-Dichloropyridin-4-yl)amino)-3-(3-hydroxy-3-methylbutyl)-1-methyl-1,3-dihydro-2H-benzo[d]imidazol-2-one; 5-((5-Chloro-2-(methylthio)pyrimidin-4-yl)amino)-3-(3-hydroxy-3-methylbutyl)-1-methyl-1,3-dihydro-2H-benzo[d]-imidazol-2-one; 5-((2,5-Dichloropyrimidin-4-yl)amino)-3-(3-hydroxy-3-methylbutyl)-1-methyl-1,3-dihydro-2H-benzo[d]imidazol-2-one; 2-Chloro-4-((cyclopropylmethyl)amino)nicotinonitrile; 3,4,2-Chloro-4-((1,3-dimethyl-2-oxo-2,3-dihydro-1H-benzo[d]-imidazol-5-yl)amino)nicotinonitrile; 2-Chloro-4-((3-(2-hydroxybutyl)-1-methyl-2-oxo-2,3-dihydro-1H-benzo[d]imidazol-5-yl)amino)nicotinonitrile; 2-Chloro-4-((3-(2-cyanobutyl)-1-methyl-2-oxo-2,3-dihydro-1H-benzo[d]imidazol-5-yl)amino)nicotinonitrile; (S)-2-Chloro-4-((3-(2-hydroxybutyl)-1-methyl-2-oxo-2,3-dihydro-1H-benzo[d]imidazol-5-yl)amino)nicotinonitrile; (R)-2-Chloro-4-((3-(2-hydroxybutyl)-1-methyl-2-oxo-2,3-dihydro-1H-benzo[d]imidazol-5-yl)amino)nicotinonitrile; 4-((3-Butyl-1-methyl-2-oxo-2,3-dihydro-1H-benzo[d]-imidazol-5-yl)amino)-2-chloronicotinonitrile; (R)-2-Chloro-4-((3-(3-hydroxybutyl)-1-methyl-2-oxo-2,3-dihydro-1H-benzo[d]imidazol-5-yl)amino)nicotinonitrile; (S)-2-Chloro-4-((3-(3-hydroxybutyl)-1-methyl-2-oxo-2,3-dihydro-1H-benzo[d]imidazol-5-yl)amino)nicotinonitrile; 1-Methyl-5-nitro-1,3-dihydro-2H-benzo[d]imidazol-2-one; 2-Chloro-4-((1-methyl-2-oxo-2,3-dihydro-1H-benzo[d]-imidazol-5-yl)amino)nicotinonitrile; 3-(2-Hydroxybutyl)-1-methyl-5-nitro-1,3-dihydro-2Hbenzo[d]imidazol-2-one; 3-Hydroxy-3-methylbutyl 4-methylbenzenesulfonate; [(3R)-3-Hydroxybutyl] 4-methylbenzenesulfonate; 3-(3-Hydroxy-3-methylbutyl)-1-methyl-5-nitro-1,3-dihydro-2H-benzo[d]imidazol-2-one; 5-Amino-3-(3-hydroxy-3-methylbutyl)-1-methyl-1,3-dihydro-2H-benzo[d]imidazol-2-one; 5-((2-Bromo-5-chloropyridin-4-yl)amino)-3-(3-hydroxy-3-methylbutyl)-1-methyl-1,3-dihydro-2H-benzo[d]imidazol-2-one; 5-Chloro-2-((3S,5R)-3,5-dimethylpiperidin-1-yl)-4-iodopyridine; (2S,6R)-4-(5-Chloro-4-iodopyridin-2-yl)-2,6-dimethylmorpholine; or various combinations thereof.

In some embodiments, the inhibitor is a small molecule compound. The inhibitor can be BI-3812 (CAS No. 2166387-64-8; 1-(5-chloro-4-((8-methoxy-1-methyl-3-(2-(methylamino)-2-oxoethoxy)-2-oxo-1,2-dihydroquinolin-6-yl)amino)pyrimidin-2-yl)-N,N-dimethylpiperidine-4-carboxamide) or an analog of BI-3812. In some embodiments, the compound is selected from the molecules disclosed in Bellenie, et al. “Achieving in vivo target depletion through the discovery and optimization of benzimidaxolone BCL6 degraders,” Journal of Medicinal Chemistry, 63:4047-4068 (2020). In some embodiments, the inhibitor is 5-((5-Chloro-2-(2,4-dimethylthiazol-5-yl)pyrimidin-4-yl)-amino)-3-(3-hydroxy-3-methylbutyl)-1-methyl-1,3-dihydro-2H-benzo[d]imidazol-2-one; 5-((5-Chloro-2-(1-methyl-1H-imidazol-2-yl)pyrimidin-4-yl)-amino)-3-(3-hydroxy-3-methylbutyl)-1-methyl-1,3-dihydro-2H-benzo[d]imidazol-2-one; 5-((5-Chloro-2-(1H-pyrazol-1-yl)pyrimidin-4-yl)amino)-3-(3-hydroxy-3-methylbutyl)-1-methyl-1,3-dihydro-2H-benzo[d]-imidazol-2-one; 5-((5-Chloro-2-(3-methyl-1H-pyrazol-1-yl)pyrimidin-4-yl)-amino)-3-(3-hydroxy-3-methylbutyl)-1-methyl-1,3-dihydro-2H-benzo[d]imidazol-2-one; 5-((5-Chloro-2-(5-methyl-1H-pyrazol-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(3,5-dimethyl-1H-pyrazol-1-yl)pyrimidin-4-yl)amino)-3-(3-hydroxy-3-methylbutyl)-1-methyl-1,3-dihydro-2H-benzo[d]imidazol-2-one; 5-((5-Chloro-2-((2R,6S)-2,6-dimethylmorpholino)pyrimidin-4-yl)amino)-3-(3-hydroxy-3-methylbutyl)-1-methyl-1,3-dihydro-2H-benzo[d]imidazol-2-one; 5-((5-Chloro-2-(2,2,6,6-tetramethylmorpholino)pyrimidin-4-yl)amino)-3-(3-hydroxy-3-methylbutyl)-1-methyl-1,3-dihydro-2H-benzo[d]imidazol-2-one; 5-((5-Chloro-2-((3S,5R)-3,4,5-trimethylpiperazin-1-yl)-pyrimidin-4-yl)amino)-3-(3-hydroxy-3-methylbutyl)-1-methyl-1,3-dihydro-2H-benzo[d]imidazol-2-one; 5-((5-Chloro-2-(3,5-dimethyl-1H-pyrazol-1-yl)pyridin-4-yl)-amino)-3-(3-hydroxy-3-methylbutyl)-1-methyl-1,3-dihydro-2H-benzo[d]imidazol-2-one; 1-(5-Chloro-4-((3-(3-hydroxy-3-methylbutyl)-1-methyl-2-oxo-2,3-dihydro-1H-benzo[d]imidazol-5-yl)amino)pyrimidin-2-yl)piperidine-3-carbonitrile; 5-((5-Chloro-2-(4-(trifluoromethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(8,8-difluoro-3-azabicyclo[3.2.1]octan-3-yl)-pyrimidin-4-yl)amino)-3-(3-hydroxy-3-methylbutyl)-1-methyl-1,3-dihydro-2H-benzo[d]imidazol-2-one; 5-((5-Chloro-2-(3,3-difluoro-8-azabicyclo[3.2.1]octan-8-yl)-pyrimidin-4-yl)amino)-3-(3-hydroxy-3-methylbutyl)-1-methyl-1,3-dihydro-2H-benzo[d]imidazol-2-one; or various combinations thereof. In some embodiments, the inhibitor is 5-((5-Chloro-2-(dimethylamino)pyrimidin-4-yl)amino)-3-(3-hydroxy-3-methylbutyl)-1-methyl-1,3-dihydro-2H-benzo[d]-imidazol-2-one; 5-((5-Chloro-2-morpholinopyrimidin-4-yl)amino)-3-(3-hydroxy-3-methylbutyl)-1-methyl-1,3-dihydro-2H-benzo[d]-imidazol-2-one; 5-((5-Chloro-2-(piperidin-1-yl)pyrimidin-4-yl)amino)-3-(3-hydroxy-3-methylbutyl)-1-methyl-1,3-dihydro-2H-benzo[d]-imidazol-2-one; 5-((5-Chloro-2-((2R,6S)-2,6-dimethylmorpholino)pyridin-4-yl)amino)-3-(3-hydroxy-3-methylbutyl)-1-methyl-1,3-dihydro-2H-benzo[d]imidazol-2-one; 2-Chloro-4-((3-(3-hydroxy-3-methylbutyl)-1-methyl-2-oxo-2,3-dihydro-1H-benzo[d]imidazol-5-yl)amino)nicotinonitrile; 5-((2,3-Dichloropyridin-4-yl)amino)-3-(3-hydroxy-3-methylbutyl)-1-methyl-1,3-dihydro-2H-benzo[d]imidazol-2-one; 5-((3-Chloropyridin-4-yl)amino)-3-(3-hydroxy-3-methylbutyl)-1-methyl-1,3-dihydro-2H-benzo[d]imidazol-2-one; 4-Chloro-6-((3-(3-hydroxy-3-methylbutyl)-1-methyl-2-oxo-2,3-dihydro-1H-benzo[d]imidazol-5-yl)amino)pyrimidine-5-Carbonitrile; 5-((5,6-Dichloropyrimidin-4-yl)amino)-3-(3-hydroxy-3-methylbutyl)-1-methyl-1,3-dihydro-2H-benzo[d]imidazol-2-one; 5-((3,5-Dichloropyridin-4-yl)amino)-3-(3-hydroxy-3-methylbutyl)-1-methyl-1,3-dihydro-2H-benzo[d]imidazol-2-one; 5-((5-Chloro-2-(methylthio)pyrimidin-4-yl)amino)-3-(3-hydroxy-3-methylbutyl)-1-methyl-1,3-dihydro-2H-benzo[d]-imidazol-2-one; 5-((2,5-Dichloropyrimidin-4-yl)amino)-3-(3-hydroxy-3-methylbutyl)-1-methyl-1,3-dihydro-2H-benzo[d]imidazol-2-one; 2-Chloro-4-((cyclopropylmethyl)amino)nicotinonitrile; 3,4,2-Chloro-4-((1,3-dimethyl-2-oxo-2,3-dihydro-1H-benzo[d]-imidazol-5-yl)amino)nicotinonitrile; 2-Chloro-4-((3-(2-hydroxybutyl)-1-methyl-2-oxo-2,3-dihydro-1H-benzo[d]imidazol-5-yl)amino)nicotinonitrile; 2-Chloro-4-((3-(2-cyanobutyl)-1-methyl-2-oxo-2,3-dihydro-1H-benzo[d]imidazol-5-yl)amino)nicotinonitrile; (S)-2-Chloro-4-((3-(2-hydroxybutyl)-1-methyl-2-oxo-2,3-dihydro-1H-benzo[d]imidazol-5-yl)amino)nicotinonitrile; (R)-2-Chloro-4-((3-(2-hydroxybutyl)-1-methyl-2-oxo-2,3-dihydro-1H-benzo[d]imidazol-5-yl)amino)nicotinonitrile; 4-((3-Butyl-1-methyl-2-oxo-2,3-dihydro-1H-benzo[d]-imidazol-5-yl)amino)-2-chloronicotinonitrile; (R)-2-Chloro-4-((3-(3-hydroxybutyl)-1-methyl-2-oxo-2,3-dihydro-1H-benzo[d]imidazol-5-yl)amino)nicotinonitrile; (S)-2-Chloro-4-((3-(3-hydroxybutyl)-1-methyl-2-oxo-2,3-dihydro-1H-benzo[d]imidazol-5-yl)amino)nicotinonitrile; 1-Methyl-5-nitro-1,3-dihydro-2H-benzo[d]imidazol-2-one; 2-Chloro-4-((1-methyl-2-oxo-2,3-dihydro-1H-benzo[d]-imidazol-5-yl)amino)nicotinonitrile; 3-(2-Hydroxybutyl)-1-methyl-5-nitro-1,3-dihydro-2Hbenzo[d]imidazol-2-one; 3-Hydroxy-3-methylbutyl 4-methylbenzenesulfonate; [(3R)-3-Hydroxybutyl] 4-methylbenzenesulfonate; 3-(3-Hydroxy-3-methylbutyl)-1-methyl-5-nitro-1,3-dihydro-2H-benzo[d]imidazol-2-one; 5-Amino-3-(3-hydroxy-3-methylbutyl)-1-methyl-1,3-dihydro-2H-benzo[d]imidazol-2-one; 5-((2-Bromo-5-chloropyridin-4-yl)amino)-3-(3-hydroxy-3-methylbutyl)-1-methyl-1,3-dihydro-2H-benzo[d]imidazol-2-one; 5-Chloro-2-((3S,5R)-3,5-dimethylpiperidin-1-yl)-4-iodopyridine; (2S,6R)-4-(5-Chloro-4-iodopyridin-2-yl)-2,6-dimethylmorpholine; or various combinations thereof.

### Screening Methods

In some aspects, the invention provides a method for screening for agents effective in promoting the degradation of a polypeptide. In various embodiments, the polypeptide comprises a degron. In some embodiments, the polypeptide comprises a polymerizable domain. The polymerizable domain can be a Broad complex/Tramtrack/Bric-a-brac (BTB) domain or derived from a BTB domain. Thus, the screen can be used to identify agents enhancing degradation of a BTB domain-containing polypeptide. In some embodiments the tertiary structure of the polymerizable domain comprises internal symmetry. In some embodiments, the polypeptide is a fusion polypeptide. In some embodiments, the fusion polypeptide comprises a fluorescent polypeptide (e.g., green fluorescent peptide or mCherry) In some embodiments, if the agent is effective in promoting degradation of the polypeptide, the agent is effective in treating or preventing a disease. In some embodiments, the polypeptide comprises a sequence derived from a marker, wherein the sequence derived from the marker can comprise a polymerizable domain. The agent can be a chemotherapeutic agent. In some embodiments, agents effective in promoting the degradation of the polypeptide also induce polymerization of the polypeptide. In some embodiments, polymerization of the polypeptide results in the formation of foci. In some embodiments, polymerization of the polypeptide enhances degradation of the polypeptide. In some embodiments, the polypeptide comprises a degron. In some embodiments, the polypeptide is degraded by the proteasome. In some embodiments, the polypeptide comprises the recombinant polypeptide described above. In some embodiments the method includes determining that the polypeptide can be degraded by the proteasome. In some embodiments, the marker is B-cell lymphoma 6 (BCL6) polypeptide. The screening method can be used to identify agents effective in promoting the degradation and/or polymerization of B-cell lymphoma 6 (BCL6) polypeptide. The screening method can be carried out in vitro or in vivo.

The method can comprise detecting the level of the fusion polypeptide contacted with the agent relative to the level of fusion polypeptide present in a corresponding control cell. In some embodiments, the control cell is a cell that has not been contacted with the agent. Reduction in the level of fusion polypeptide in the cell relative to the control cell can identify the agent as effective in inducing degradation of the polypeptide. Detection may be achieved using imaging techniques or by monitoring fluorescence.

In some embodiments, the method comprises contacting a cell expressing the polypeptide with the agent. The method can further comprise imaging the cell after contacting the cell with the agent, optionally using fluorescence microscopy, fluorescence-activated cell sorting, imaging flow cytometry, or any of various other high-throughput imaging techniques. The method can comprise immunostaining the polypeptide or detecting a fluorescent signal generated by the polypeptide to determine whether contacting the cell with the agent resulted in polymerization and/or degradation of the polypeptide. In some embodiments, the method comprises imaging foci formed through polymerization of the polypeptide induced by the agent. The method can include selecting an agent from the screen capable of inducing polymerization and/or degradation of the polypeptide as a candidate for treating a disease associated with the marker. The method can include selecting an agent from the screen that resulted in the formation of foci.

In some embodiments, the method includes expressing the polypeptide in a cell using a stability vector. A non-limiting example of a stability vector suitable for use in the methods of the invention is shown in FIG. 1C. The method can include exposing a plurality of cells expressing the polypeptide to a plurality of agents to identify agents capable of inducing polymerization of the polypeptide. In some embodiments, polymerization is detected through imaging of foci.

### Methods for Treatment and Diagnosis

In some aspects, the invention provides a method for selecting a subject for treatment with a compound that induces BCL6 degradation. The cell can be in a subject. The subject can be a human. In some aspects, the invention provides a method for reducing proliferation of a cancer or tumor cell. In some aspects, the invention provides a method for predicting whether a subject will be resistant or sensitive to treatment with an agent, optionally a chemotherapeutic agent (e.g., BI-3802 or any other agent for treating a cancer or preventing or reducing proliferation of a cell). In some aspects, the invention provides a method for selecting a subject for treatment with an agent. In various embodiments, the subject is selected for treatment with the agent if the subject is predicted to be sensitive to the agent. Subjects having certain mutations are resistant to treatment with a compound described herein (e.g., BI-3802). In particular, subjects having an alteration at amino acid at R28, E41, C84, G55, or Y58 of BCL6 are resistant to treatment with an agent that induces degradation of BCL6 (e.g., BI-3802). In some embodiments, the subject has cancer or a tumor. In some embodiments, the subject has a B-cell lymphoma cell, a SuDHL4 cell, a Non-Hodgkin lymphoma cell, a diffuse large B cell lymphoma (DLBCL) cell, a follicular lymphoma cell, or a HEK293T cell.

In various embodiments, the agent can enhance degradation of a polypeptide. In some embodiments, the polypeptide is targeted by the agent. The polypeptide can be B-cell lymphoma 6 (BCL6) polypeptide. The method can include sequencing a portion of a gene encoding the polypeptide. The method can include detecting in a biological sample of the subject the presence or absence of an alteration at amino acid at R28, E41, C84, G55, or Y58 of BCL6. The gene can be a marker gene. The method can include predicting that a subject or cell will be resistant to treatment with the agent if a Broad complex/Tramtrack/Bric-a-brac (BTB) domain comprised by the polypeptide comprises an alteration at one of, any of, or all of R28, E41, C84, G55, and Y58. The method can include predicting that the subject or cell will be sensitive to the agent if a Broad complex/Tramtrack/Bric-a-brac (BTB) domain comprised by the polypeptide does not comprise an alteration at one of, any of, or a combination of a R28, E41, C84, G55, and Y58. In some embodiments, the alteration is a mutation to alanine. Absence of an alteration at R28, E41, C84, G55, or Y58 can select the subject for treatment with the agent. The method can include determining that the subject or cell expresses the polypeptide and that degrading the polypeptide will result in effective treatment of a disease suffered by the subject or mediated by the cell. If the subject or cell does not express the polypeptide, the subject or cell will be resistant to treatment with the agent. In some embodiments, the agent is BI-3802 or an analog thereof. The method can include predicting that a subject or cell will be resistant to treatment with the agent if a Broad complex/Tramtrack/Bric-a-brac (BTB) domain comprised by the polypeptide does not comprise a degron. The method can include predicting that a subject or cell will be sensitive to treatment with the agent if a Broad complex/Tramtrack/Bric-a-brac (BTB) domain comprised by the polypeptide comprises a degron. The method can include predicting that a subject or cell will be sensitive to treatment with the agent if the polypeptide comprises a degron.

The method can include contacting a cell, optionally a cell obtained from a subject, with the agent and determining, optionally through cell imaging, whether the agent induced polymerization of a polypeptide to be degraded. The method can include predicting that if the agent induced polymerization of the polypeptide in the cell that the cell will be sensitive to treatment with the agent. In some embodiments, polymerization of the polypeptide results in the formation of foci in a cell. The method can include predicting that a cell is sensitive to the agent if contacting the cell with the agent results in the formation of foci in the cell comprising the polypeptide. The method can include predicting that a subject will be sensitive to the agent if cells derived from the subject form foci comprising the polypeptide when exposed to the agent. The cell can be a disease-associated cell, such as, to provide non-limiting examples thereof, a B-cell lymphoma cell, a SuDHL4 cell, a Non-Hodgkin lymphoma cell, a diffuse large B cell lymphoma (DLBCL) cell, a follicular lymphoma cell, or a HEK293T cell.

The method can include determining if a marker gene encoding a polypeptide targeted by a compound and comprised by a genome of the tumor or cancer cell encodes a Broad complex/Tamtrack/Brick-a-brack (BTB) domain comprising amino acid residues R28, E41, C84, G55, and Y58. The method can include determining if the marker gene encodes a degron. The method can also include contacting the cancer or tumor cell with the compound only if it is determined that 1) the marker gene encodes a BTB domain comprising amino acid residues R28, E41, C84, G55, and Y58 and 2) the marker gene encodes a degron.

The method can include determining a sequence of the marker gene. A sequence of the marker gene can be determined using direct sequencing, DNA hybridization, and/or restriction enzyme digestion methods. DNA hybridization methods include the use of DNA microchips or DNA microarrays. A sequence of the marker gene can be determined using any of various methods available in the art including, but not limited to, shotgun sequencing, Maxam-Gilbert sequencing, or chain-termination methods. High throughput methods can be used to determine a sequence of the marker gene. Non-limiting examples of high throughput methods for DNA sequencing include single-molecule real-time sequencing, ion semiconductor-based sequencing (e.g., Ion Torrent sequencing), pyrosequencing, sequencing by synthesis (e.g., ILLUMINA sequencing), combinatorial probe anchor synthesis, sequencing by ligation, nanopore sequencing, GenapSys sequencing, and chain-termination sequencing. Specific examples of high-throughput DNA sequencing technologies include, but are not limited to, Pacific Biosciences sequencing, 454 sequencing, Illumina sequencing, cPAS-BGI/MGI sequencing, SOLid sequencing, Sanger sequencing, massively parallel signature sequencing (MPSS), polony sequencing, combinatorial probe anchor synthesis (cPAS), DNA nanoball sequencing, tunneling currents DNA sequencing, sequencing by hybridization, sequencing with mass spectrometry, RNA polymerase-based sequencing, in vitro virus high-throughput sequencing, and heliscope molecule sequencing. Various methods in microfluidics can be used to determine a sequence of the marker gene (e.g., microfluidic Sanger sequencing). Microscopy-based methods can also be used to determine a sequence of the marker gene.

In some aspects, the invention provides a method for treating a subject with an agent. The method includes administering the agent to the subject only if the subject is predicted to be sensitive to the agent and not resistant to the agent.

### Imaging and Efficacy Measurement Methods

In some aspects, the invention provides for a method of measuring efficacy of an agent in inducing degradation of a polypeptide expressed by a cell. The polypeptide can be a fusion polypeptide comprising a BTB domain. In some embodiments, the polypeptide is a pharmacodynamic marker. In some aspects, the invention provides a method for imaging a cell using a pharmacodynamic marker comprising the fusion polypeptide or a polymerizable polypeptide (e.g., the fusion polypeptides or recombinant polypeptides described above). In some embodiments, the method includes detecting foci formed within the cell after exposure to an agent. The agent can induce polymerization of the pharmacodynamic marker or polypeptide. In various embodiments, the pharmacodynamic marker comprises the fusion polypeptide or recombinant polypeptide described above. In some embodiments, detecting foci comprises imaging a cell and/or imaging foci. The agent can be a compound, including one of those described above. The agent can be BI-3802 or an analog thereof. The polypeptide can be BCL6.

The method can comprise detecting the level of the fusion polypeptide contacted with the agent relative to the level of fusion polypeptide present in a corresponding control cell. In some embodiments, the control cell is a cell that has not been contacted with the agent. Reduction in the level of fusion polypeptide in the cell relative to the control cell can identify the agent as effective in inducing degradation of the polypeptide.

The method can include contacting a cell expressing the fusion polypeptide or polymerizable polypeptide with the agent and subsequently detecting foci present within the cell. The cell can be any of the cells described above. The cell can be a cancer cell, non-limiting examples of which include a B-cell lymphoma cell, a SuDHL4 cell, a Non-Hodgkin lymphoma cell, a diffuse large B cell lymphoma (DLBCL) cell, a follicular lymphoma cell, or a HEK293T cell.

The method can include labeling the polypeptide with a label. In some embodiments, if the agent is effective in inducing degradation of the polypeptide, then foci comprising the polypeptide will form in the cell after the cell is contacted with the agent. In some embodiments, the foci comprise the fusion polypeptide or polymerizable polypeptide. The fusion polypeptide or polymerizable polypeptide can be imaged using a label. In some embodiments, the fusion polypeptide comprises a fluorescent polypeptide as a label. In some embodiments, the label is an immunofluorescent dye that specifically binds the fusion polypeptide or polymerizable polypeptide. The label can be imaged by techniques in fluorescence microscopy.

In some embodiments, the method can be used to track efficacy of an agent during treatment of a subject. The treatment of the subject can comprise administering to the subject the agent. The treatment can be a cancer treatment.

The label can be detected or imaged using fluorescence-activated cell sorting, or imaging flow cytometry. The label can comprise a fluorophore. The label can be an immunofluorescent molecule.

### Compositions

In an aspect, the invention provides for compositions comprising a recombinant nucleotide molecule encoding a fusion polypeptide, as described above. In some aspects, the invention provides for compositions comprising the fusion polypeptide, the recombinant polypeptide, the cell, the expression vector, or the recombinant nucleotide molecule described above.

Useful solvents, additives, and diluents for the preparation of the compositions hereof, can be solids, liquids, or gases. These include any and all solvents, dispersion media, coatings, antibacterial and antifungal agents, isotonic and absorption delaying agents and the like. The solvent, additive, or diluent does not destroy biological functionality of the disclosed compound. Thus, the compositions can take the form of tablets, powders, solutions, or suspensions. Water, saline, aqueous dextrose, and glycols are examples of liquids suitable for use in the compositions. Suitable additives include sodium chloride, glycerol, propylene glycol, water, and ethanol. The compositions may include additives such as preservatives, stabilizing agents, wetting or emulsifying agents, salts for adjusting osmotic pressure, and buffers. Such compositions will, in any event, contain an effective amount of the polypeptide or nucleotide molecule for use in a target application.

In some embodiments, the pH of the composition is from 4.0 to 8. In other embodiments, the pH of the composition is from 6.0 to 8. In another embodiment the composition has a pH of about 7. These pH ranges may be achieved through the incorporation of one or more pH modifying agents, buffers, and the like.

### Biomaterials

In some aspects, the present invention provides for biomaterials manufactured by polymerizing the fusion polypeptides described above. The fusion polypeptides can be polymerized by any of the methods described herein. The fusion polypeptides can be polymerized in vitro outside of a cell. The fusion polypeptides can be polymerized in a cell.

Biomaterials can be defined as fusion protein polymers and/or material matrices comprising the fusion protein polymers of the present invention, wherein the fusion protein polymers are optionally formed according to the methods described herein. The biomaterials can be used for a therapeutic or diagnostic purpose. The biomaterials can be used to coat and/or functionalize a surface.

The biomaterial can be made through the polymerization of one or a plurality of fusion proteins, wherein the plurality of fusion proteins may be unique fusion proteins each comprising a unique heterologous peptide. The biomaterial can be a mosaic protein polymer. In certain applications, it can be useful to coat a surface with biomaterials of the present invention to functionalize the surface (e.g., with an enzymatic or antigen-binding activity). The biomaterials can be engineered to have different functionalities based upon the heterologous proteins to which the fusion proteins are linked. In some embodiments, a fusion proteins of the present invention may comprise a plurality of BTB domains to facilitate biomaterial formation.

### Kits

In an aspect, the invention provides kits for use in the above described methods for predicting whether a subject or cell will be sensitive to treatment with an agent that can enhance degradation of a polypeptide. In some aspects, the invention provides kits for use in the above described methods for screening for agents capable of inducing polymerization of a polypeptide. If desired a kit includes any one or more of the following: a nucleotide construct encoding a fusion polypeptide and/or a Broad complex/Tramtrack/Bric-a-brac (BTB) domain, nucleotide molecules encoding fluorescent polypeptides, cells, DNA or RNA polymerase, an agent(s) to be screened or used in evaluation of sensitivity of a cell or subject to treatment therewith, primers for use in gene sequencing, a list of gene mutations indicative of cell or subject resistance to treatment with the agent, labels (e.g., labeled antibodies), or various combinations thereof.

The kits may include instructions for assays or screens, reagents, testing equipment (test tubes, reaction vessels, needles, syringes, multi-well plates, fluorescence-activated cell sorting equipment, image flow cytometry equipment, etc.), standards for calibrating the assays or screens, and/or equipment provided or used to conduct the assays or screens. The instructions provided in a kit according to the invention may be directed to suitable operational parameters in the form of a label or a separate insert. Optionally, the kit may further comprise a standard or control information so that the test sample can be compared with the control information standard to determine whether a consistent result is achieved.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: BI-3802 Induces Specific BCL6 Degradation

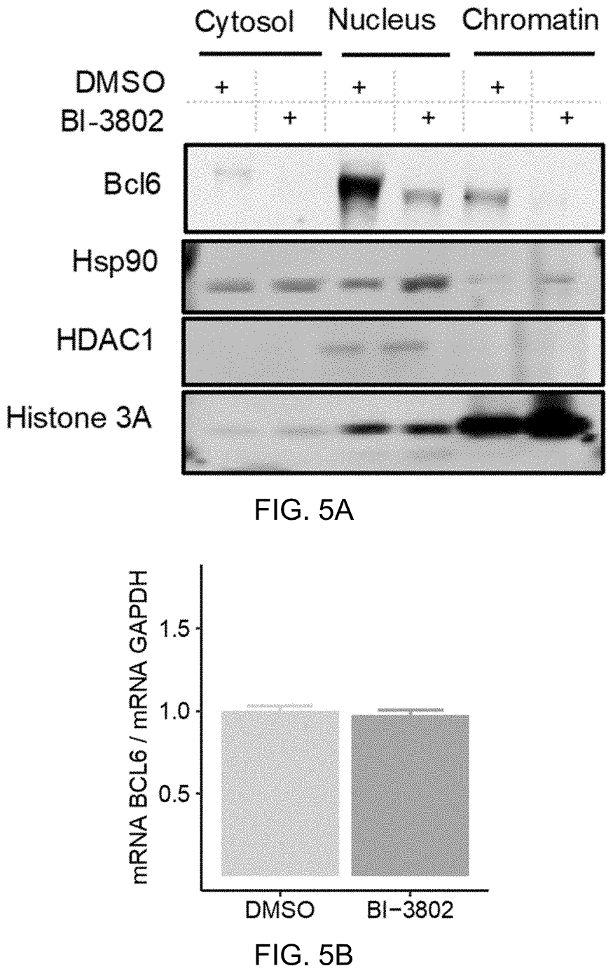

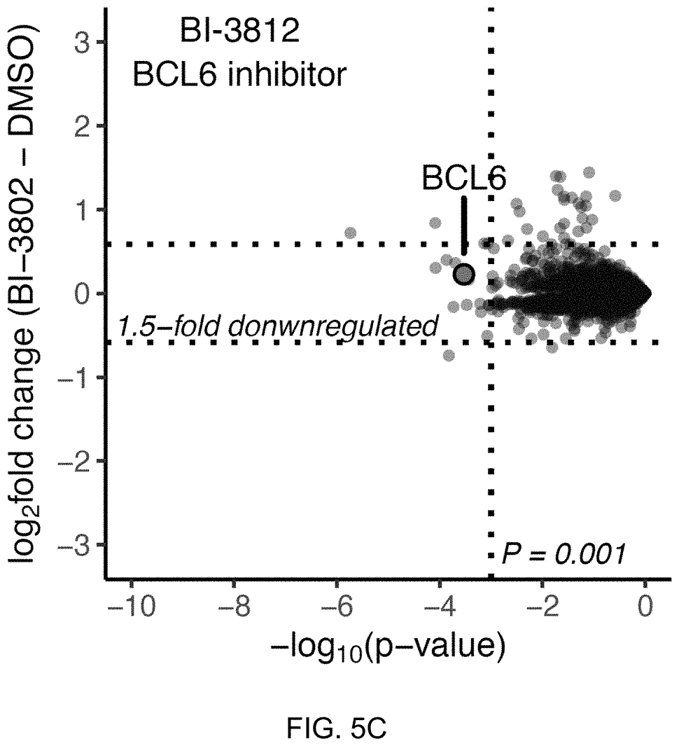

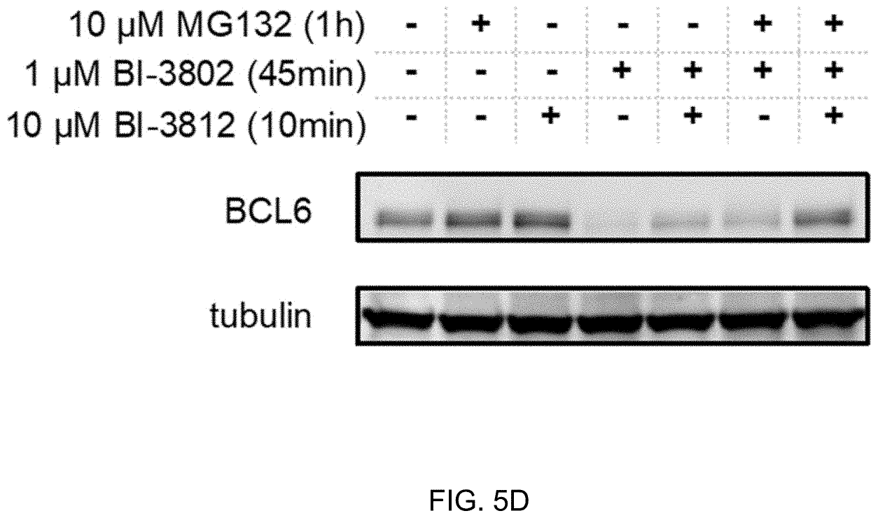

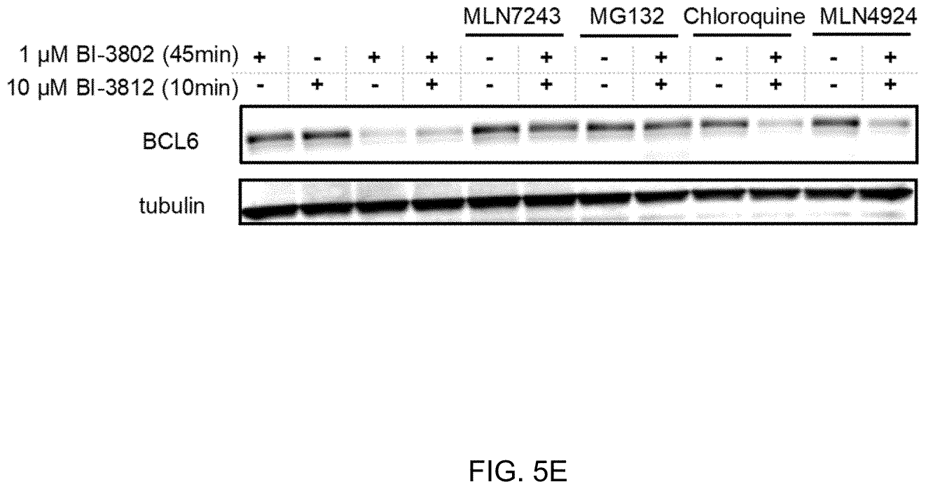

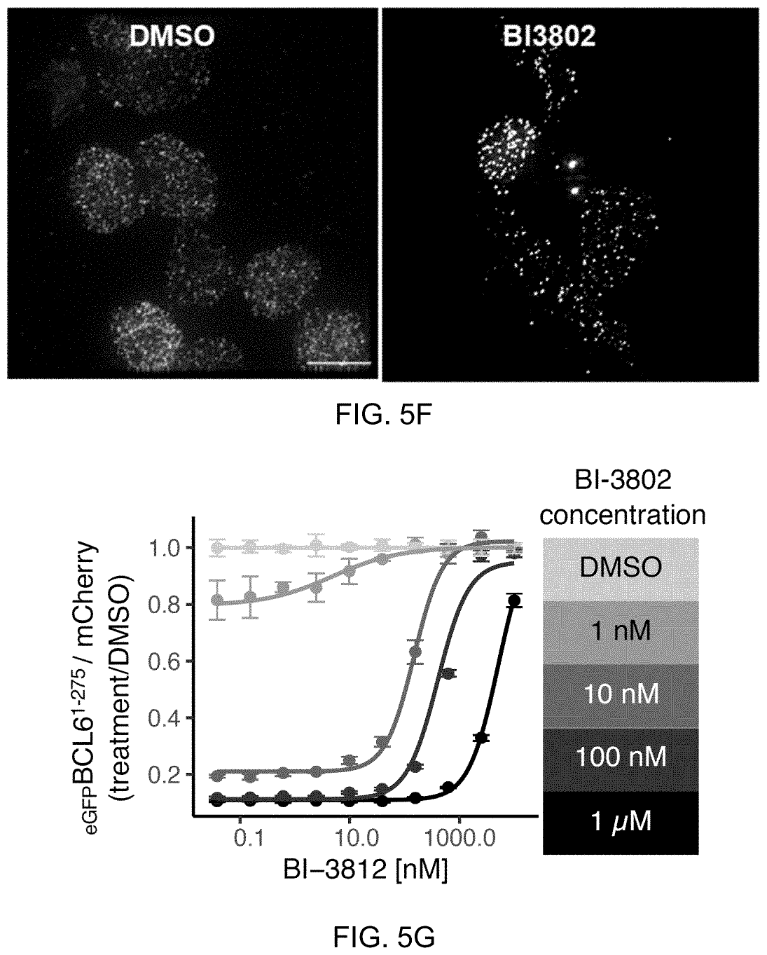

To determine the specificity of BI-3802 as a compound inducing enhanced degradation of BCL6 (FIG. 1A), quantitative mass spectrometry (MS) based proteomics was performed in SuDHL4 cells, a DLBCL-derived cell line, following compound treatment for 4 hours. BCL6 was the only polypeptide with significantly decreased abundance (FIG. 1B). BI-3802 efficiently depleted chromatin-bound BCL6 and did not alter BCL6 mRNA expression (FIGS. 5A and 5B). Treatment with the structurally similar BCL6 inhibitor BI-3812 (FIG. 1A) did not alter the abundance of any polypeptide (FIG. 5C).

To identify the critical region of BCL6 that mediates drug-induced degradation, a fluorescent reporter system was generated in HEK293T cells, in which the full length BCL6 (BCL6^{FL}) was fused in-frame with eGFP followed by an internal ribosome entry site (IRES) and mCherry (FIG. 1C). BI-3802 induced degradation of the full-length BCL6 reporter, while the inhibitor, BI-3812, did not alter stability of the reporter. BI-3802-induced degradation of _{eGFP}BCL6^{FL}, was attenuated by chemical inhibition of the 26S proteasome with MG132 or inhibition of the ubiquitin activating enzyme UBA1 by MLN7243, but not by inhibition of the neddylation pathway with MLN4924, which is required for activity of the Cullin-RING family of E3 ubiquitin ligases (FIGS. 1D, 5D, and 5E).

Analysis of stepwise C-terminal truncations of the BCL6 polypeptide in our reporter demonstrated that the first 275 amino acids, which include the drug-binding BTB domain, are sufficient for BI-3802 degradation (FIG. 1E). These studies demonstrated that BI-3802 induced selective degradation of BCL6, that degradation was mediated by a non-Cullin E3 ubiquitin ligase, and that a 275 amino acid region was sufficient for drug-dependent degradation.

### Example 2: BI-3802 Induces Cellular BCL6 Foci

Cellular localization of the BCL6-eGFP fusion construct upon exposure to BI-3802 was examined by live cell fluorescence microscopy. Strikingly, the appearance of distinct eGFP-containing foci was observed within minutes of BI-3802 treatment for both the full length BCL6 construct and the minimal degradable construct _{eGFP}BCL6¹⁻²⁷⁵ (FIG. 1F). The eGFP signal and foci subsequently disappeared, consistent with BCL6 degradation. Immunofluorescence studies in SuDHL4 cells confirmed that endogenous BCL6 also formed foci upon treatment with BI-3802 (FIG. 5F). Addition of an excess of the inhibitor BI-3812, which competes for the same site on the BCL6-BTB domain, efficiently blocked BI-3802-induced BCL6 degradation (FIG. 5G). To interrogate the dynamic of drug induced foci formation, a BTB containing, non-degradable _{eGFP}BCL6¹⁻²⁵⁰ construct was utilized that similar to wild type BCL6 formed BI-3802-induced foci that, however, persisted even after prolonged drug treatment (FIG. 1G). These drug-induced foci were fully reversible by addition of excess BI-3812 (FIG. 1G).

### Example 3: BI-3802 Induces BCL6 Polymerization

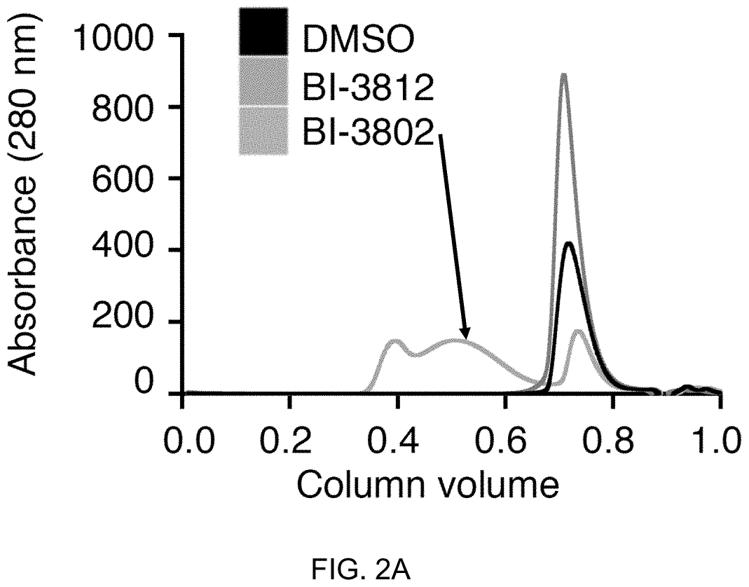

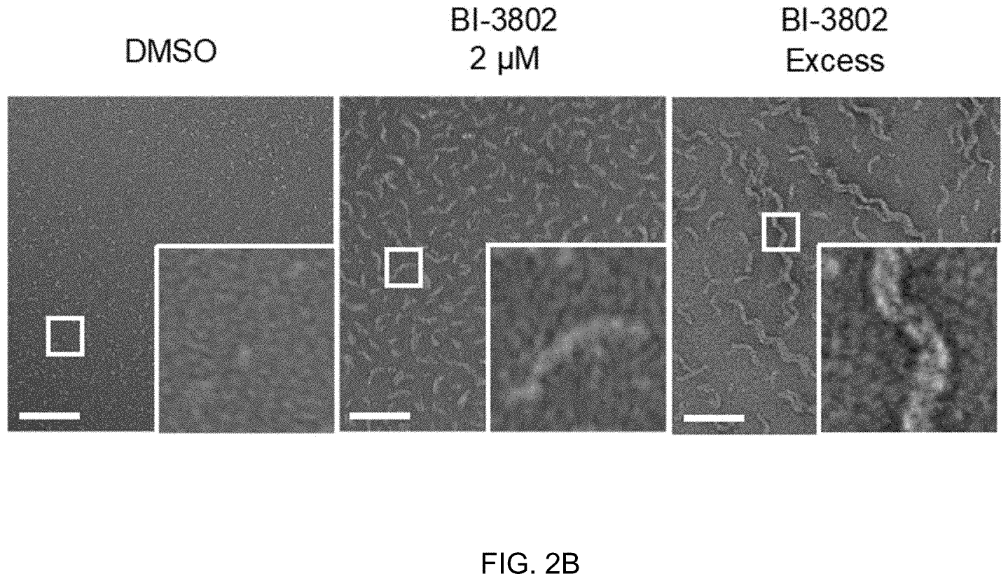

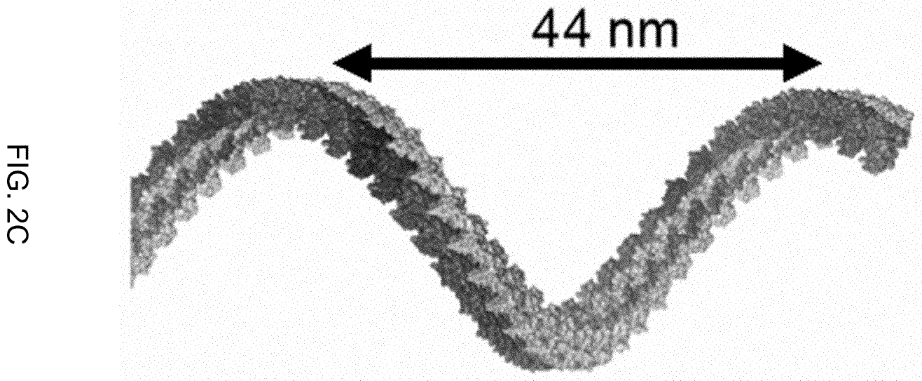

To explore the molecular basis of BCL6 foci formation, the behavior of recombinant BCL6 was examined in vitro. During purification of BCL6 recombinant polypeptide, presence of BI-3802, but not BI-3812, led to higher molecular weight species of BCL6 (FIG. 2A). Given the formation of reversible cellular foci upon BI-3802 treatment, a hypothesis was developed that BCL6 might form regular higher-order structures upon binding to BI-3802, which was examined by negative stain electron microscopy (EM). In the absence of BI-3802, BCL6 was present as monodisperse particles. However, upon incubation of BCL6 with BI-3802, the formation of regular structures with a sinusoidal shape was observed, increasing in length with higher concentration of BI-3802 (FIG. 2B).

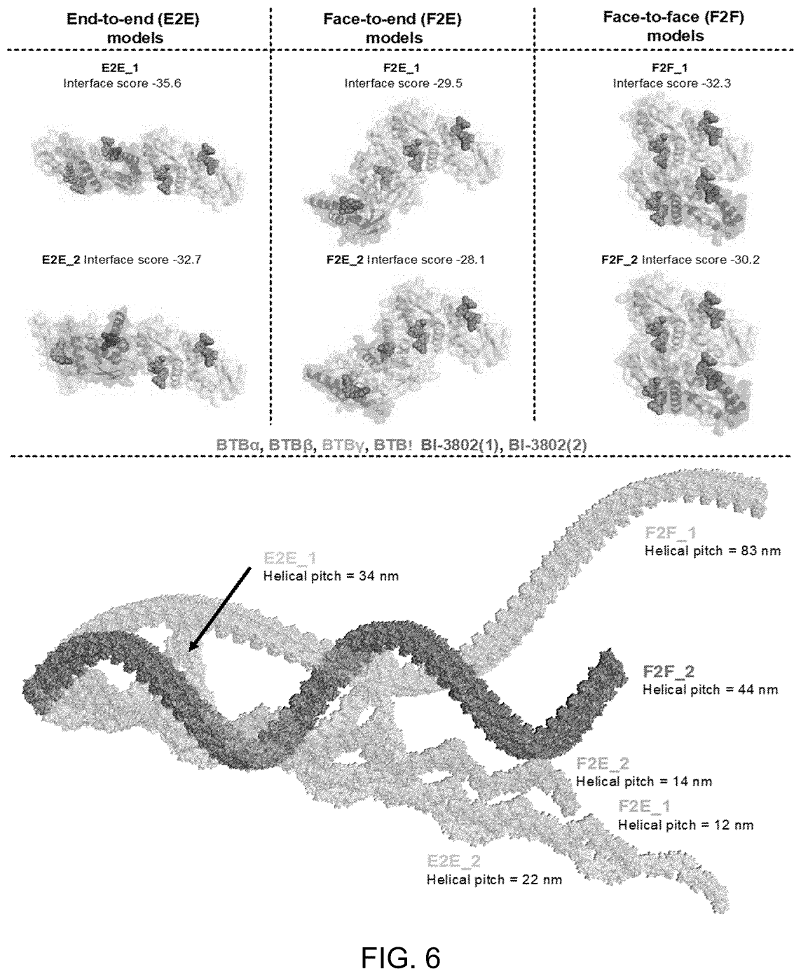

To model the filaments, two BCL6-BTB domain dimers (PDB: 5MW2) were computationally docked in the presence of BI-3802 to determine energetically favorable binding modes, and the structure was extended by sequentially aligning dimers in the same binding mode to obtain polymer models (FIG. 6). Only a symmetric association of two dimers with two molecules of BI-3802 at the interface (model number: F2F_2) gave rise to a helical superstructure approximating the pitch and the shape observed by negative stain EM (FIG. 2C).

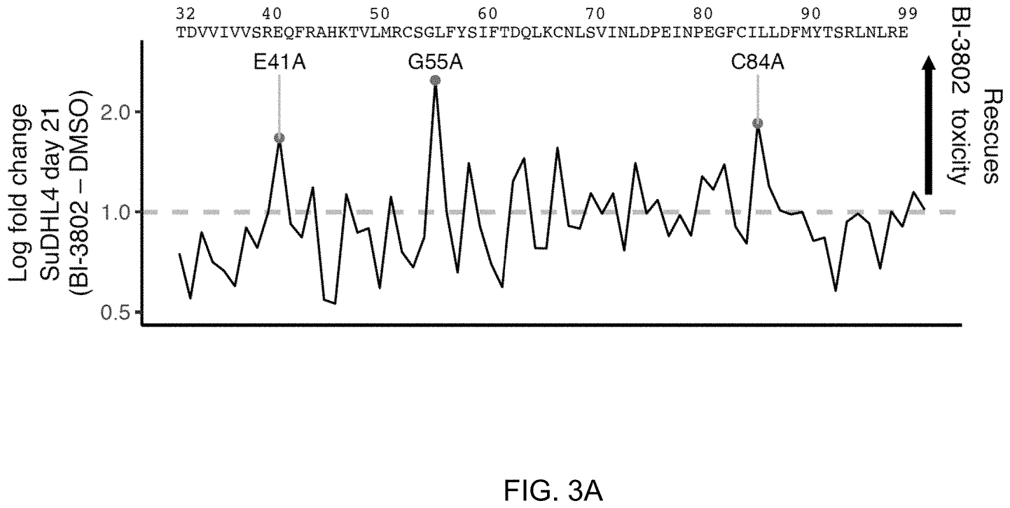

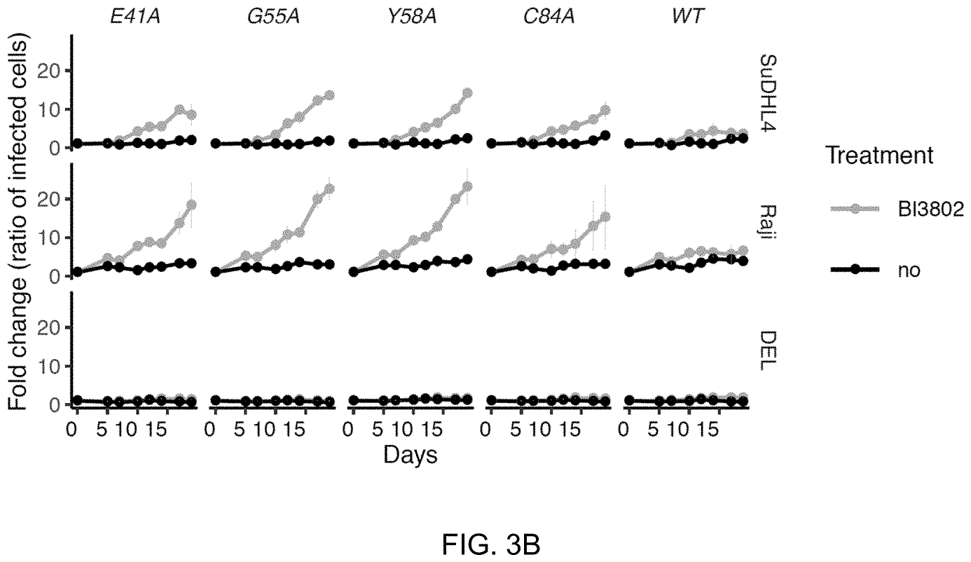

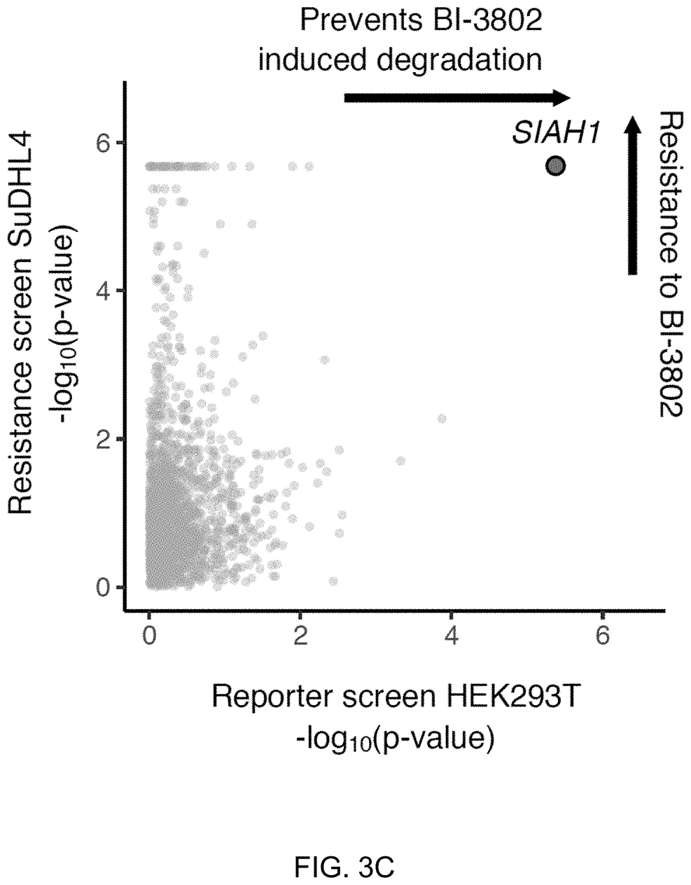

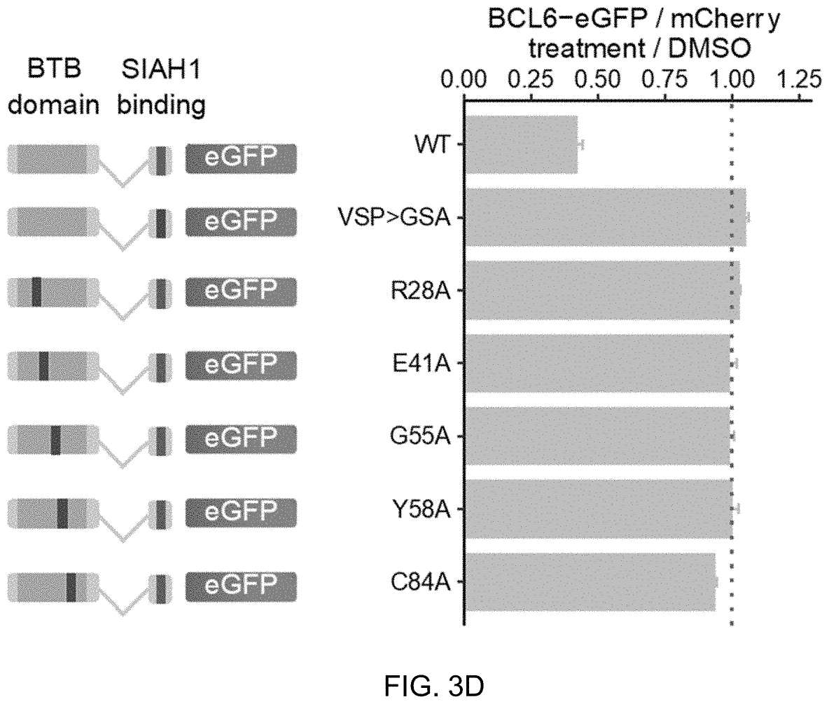

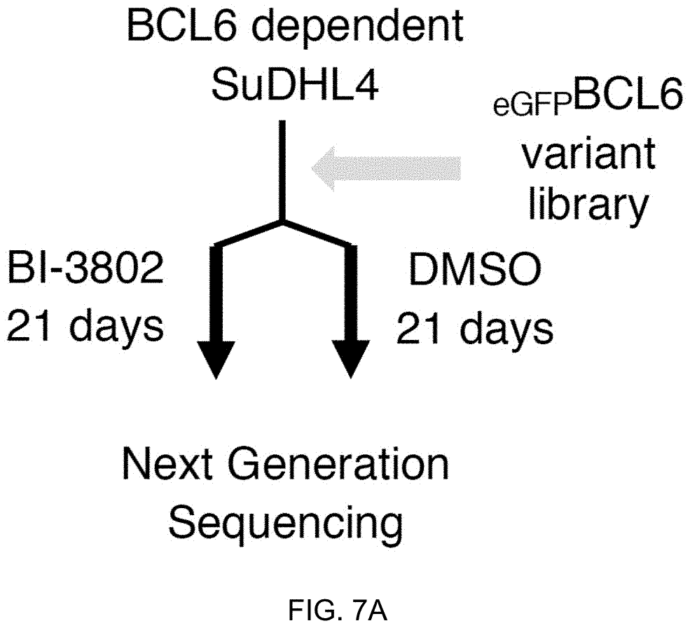

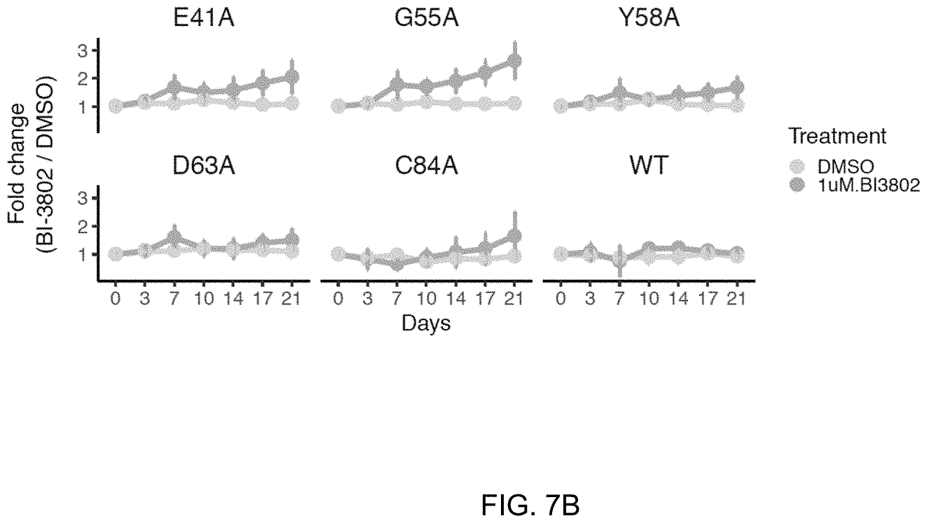

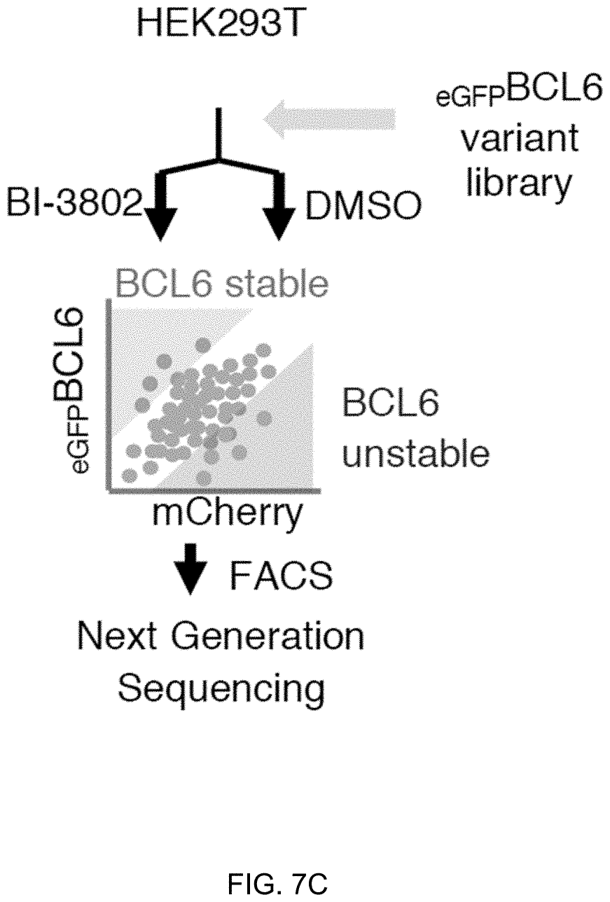

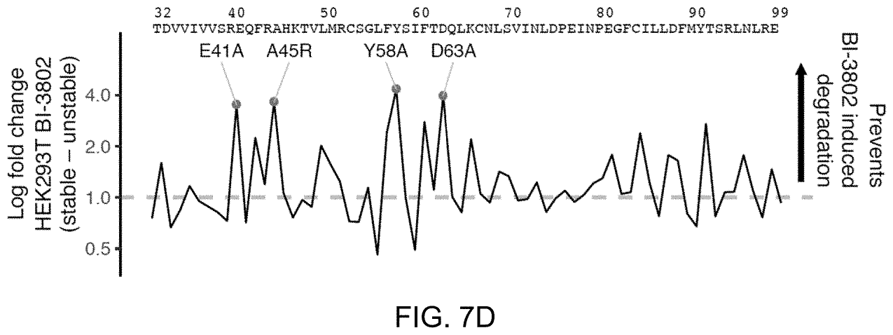

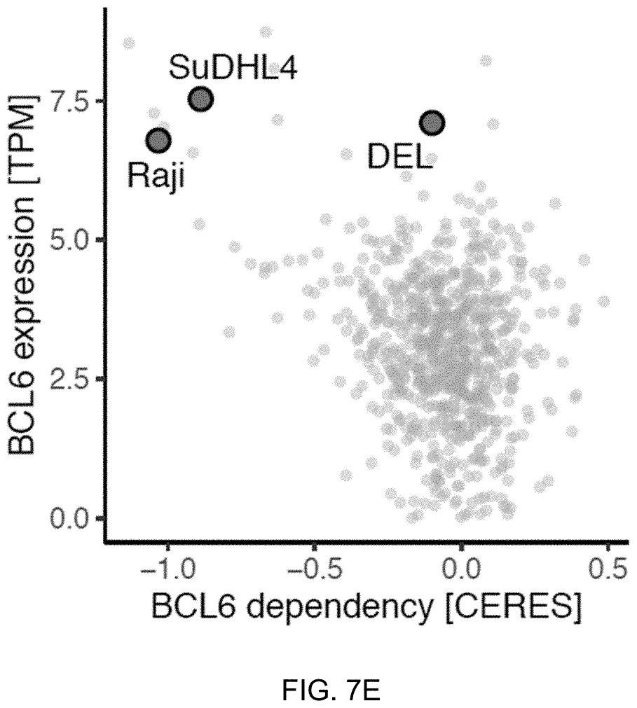

To identify key amino acids that were critical for BI-3802 activity in an unbiased fashion, a systematic alanine scan of the BTB domain (residues 32-99) was performed. The effect of each mutation on BI-3802 cellular toxicity was evaluated in SuDHL4 lymphoma cells (FIGS. 3A, 7A, and 7B) and on BI-3802-induced degradation of the BCL6 reporter in HEK293T cells (FIGS. 7C and 7D), from which we selected the top 4 residues for detailed validation (E41A, G55A, Y58A, and C84A). Overexpression of these variants in the BCL6-dependent SuDHL4 and Raji cell lines conferred resistance to BI-3802 but had no effect in the BCL6-independent DEL cell line (FIGS. 3B and 7E). BI-3802 induced polymerization of BCL6, and blocking BCL6 polymerization impaired foci formation, BCL6 degradation, and BI-3802 cellular toxicity in lymphoma cells.

### Example 4: SIAH1 is Involved in Degradation of Polymerized BCL6

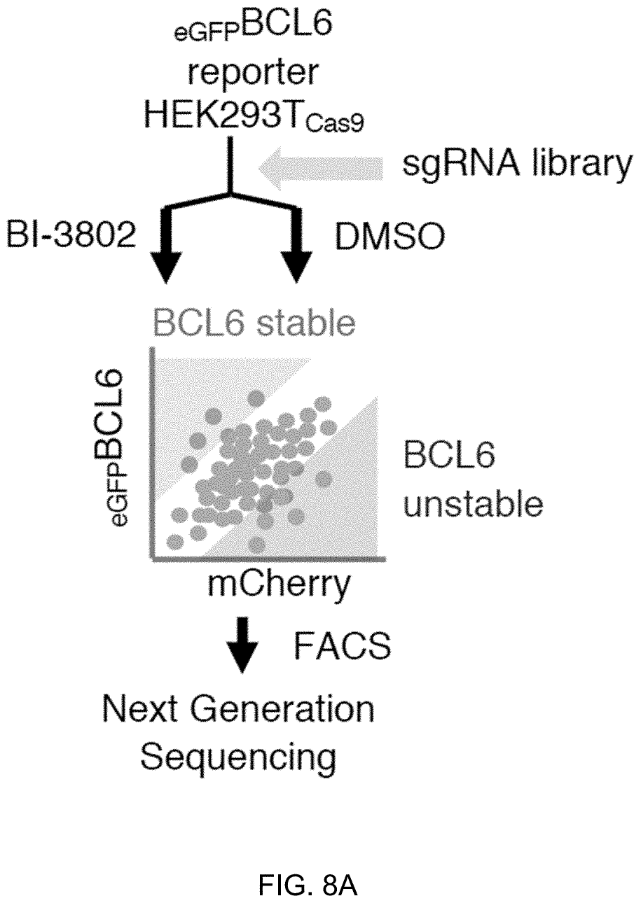

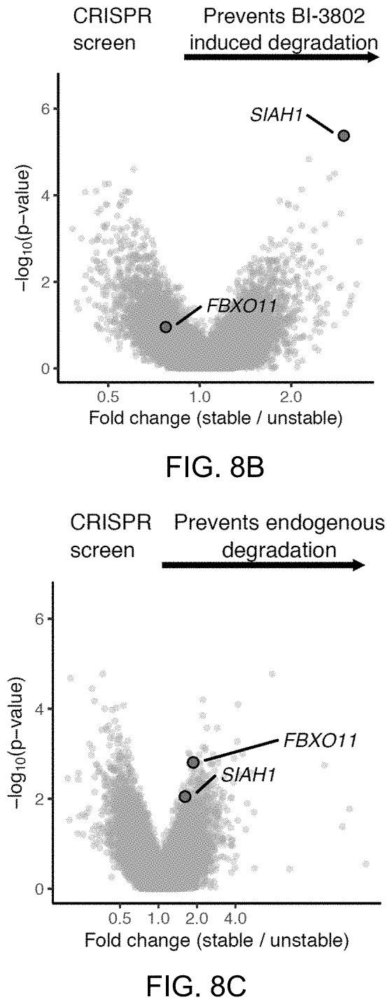

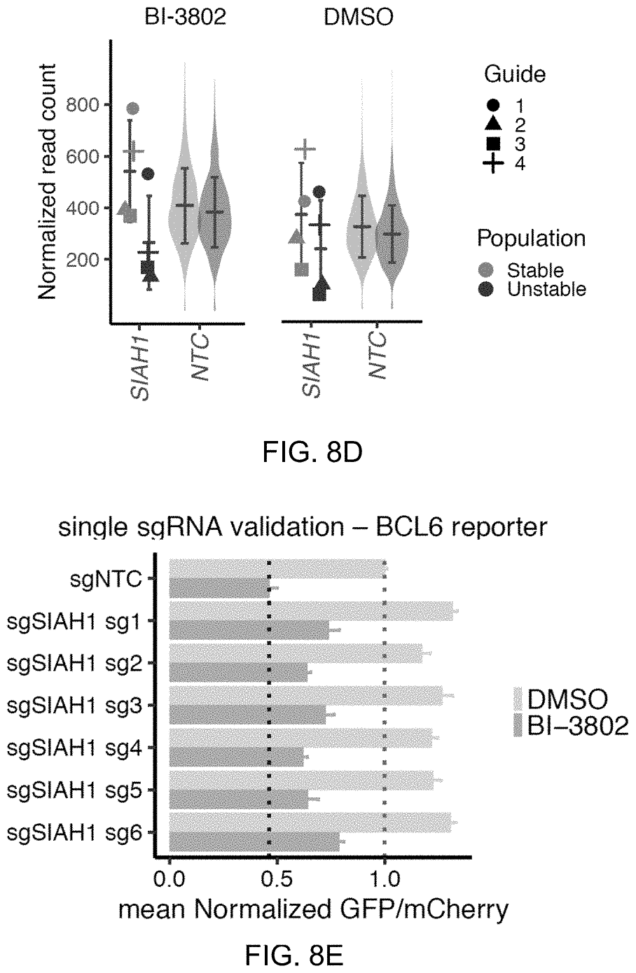

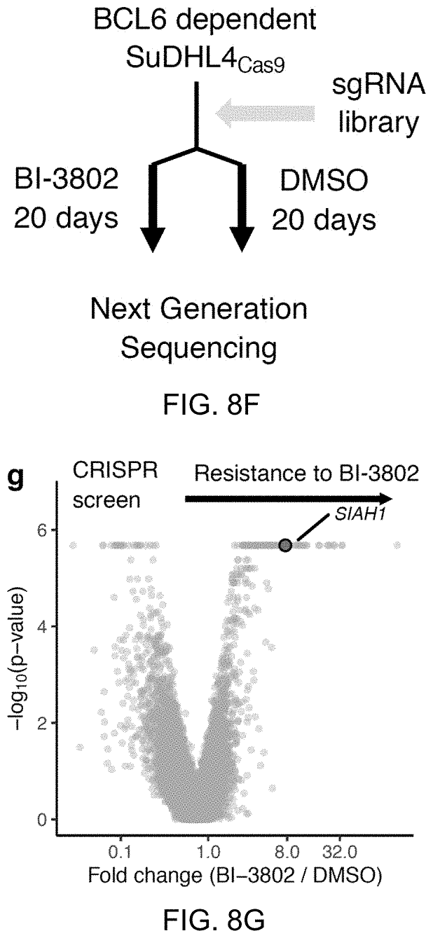

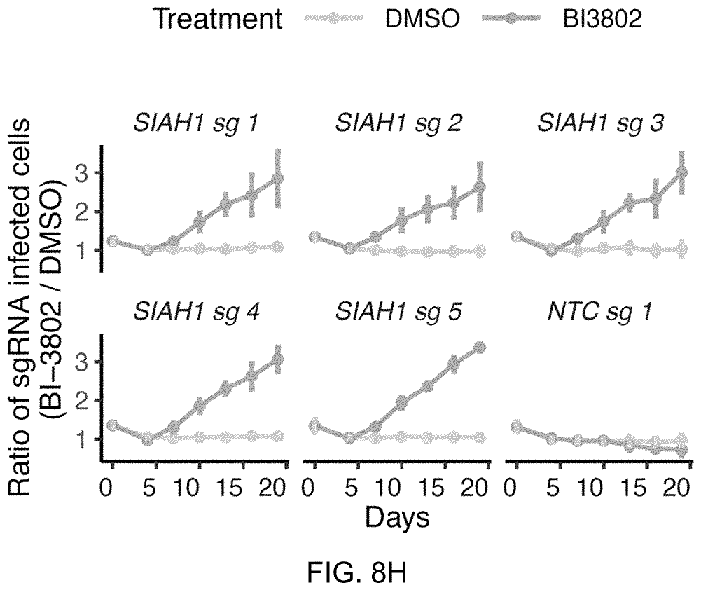

Efforts were made to discover cellular machinery necessary for BI-3802-induced BCL6 degradation. Two complementary, genome-scale CRISPR-Cas9 genetic screens were employed to interrogate the mechanism of drug-induced BCL6 degradation. First, a flow cytometry-based BCL6 reporter screen was performed in HEK293T cells, where cells infected with the sgRNA library were treated with BI-3802 or DMSO, and cell populations with increased (highest 5% eGFP/mCherry ratio) or decreased (lowest 5% eGFP/mCherry ratio) levels of _{eGFP}BCL6^{FL} were sorted from the bulk population (FIGS. 8A-8E). Second, a BI-3802 resistance screen in SuDHL4 cells (FIGS. 8F-8H) was completed. The only gene that scored significantly in both screens was the non-cullin E3 ubiquitin ligase SIAH1 (FIG. 3C).

To validate the role of SIAH1 in drug-induced BCL6 degradation and resistance to BI-3802, SIAH1 was targeted with multiple independent sgRNAs. Each sgRNA attenuated _{eGFP}BCL6^{FL} degradation upon BI-3802 treatment and induced resistance to BI-3802 treatment (FIGS. 8E and 8H). Overexpression of wild-type SIAH1 not only enhanced BI-3802-dependent BCL6 degradation, but also reduced BCL6 abundance in the absence of drug (FIG. 9A), implicating a role of SIAH1 in both drug-dependent and independent BCL6 degradation. The SIAH1 E3 ligase recognizes a V×P motif on substrate polypeptides, and this motif is present in BCL6 residues 249-251 (FIG. 9B). Not wishing to be bound by theory, deletion of the V×P motif provides an explanation for the C-terminal truncation analysis, in which BCL6¹⁻²⁷⁵ was effectively degraded in the presence of BI-3802 but BCL6¹⁻²⁵⁰ was not, despite the ability of this shorter construct to form foci in the presence of drug (FIG. 1E). Direct C-terminal fusion of the V×P-containing polypeptide (BCL6²⁴¹⁻²⁶⁰) to the BTB domain (BCL6¹⁻¹²⁹) was sufficient for BI-3802-induced degradation mediated by SIAH1 (FIG. 9C), and degradation was fully attenuated by mutation of the BCL6 V×P motif (VSP>GSA) (FIG. 3D). In this BTB-SIAH1 degron construct, mutations R28A, E41A, C84A, G55A, Y58A completely abolished BI-3802-induced degradation. Together these data demonstrated that SIAH1 may be an E3 ligase involved in BI-3802-induced BCL6 degradation.

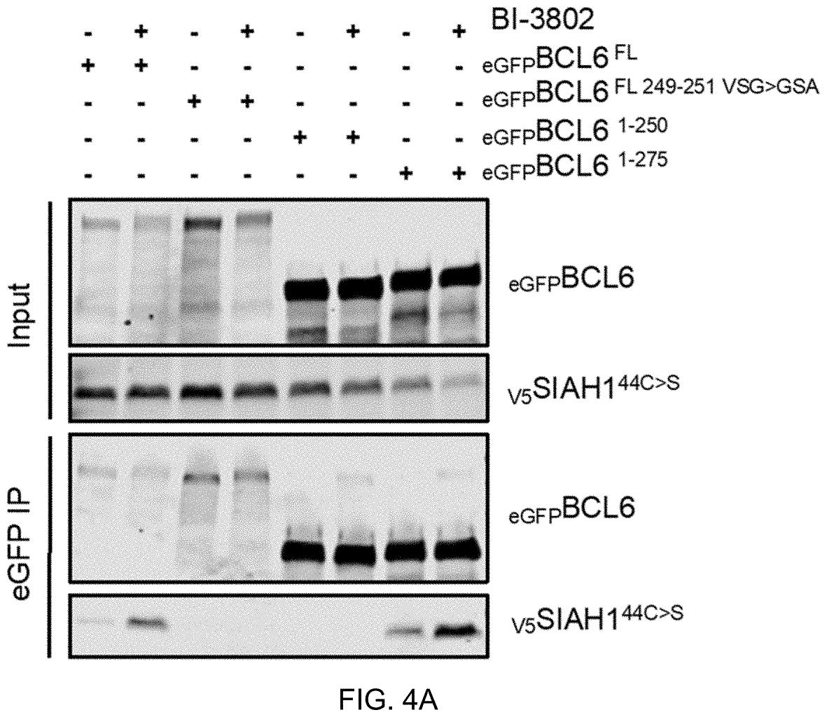

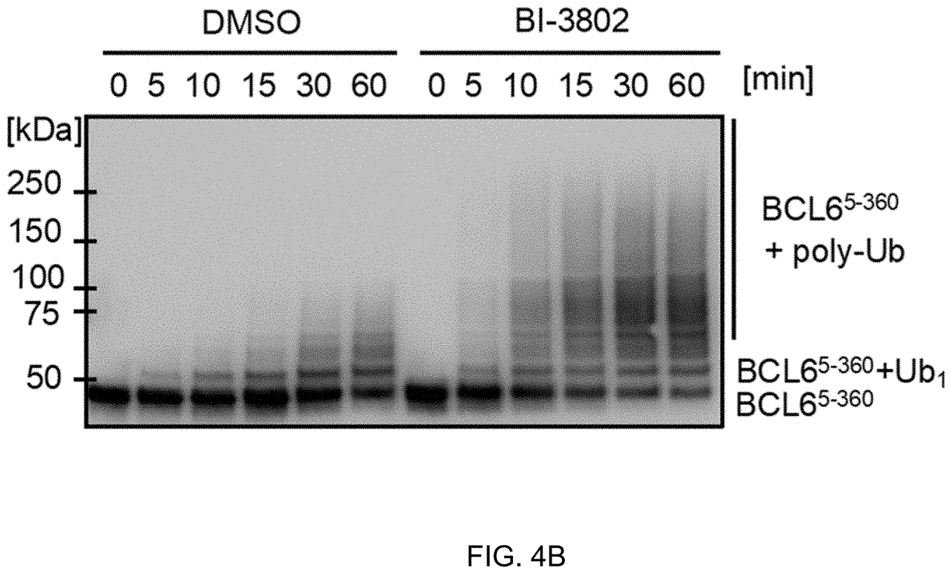

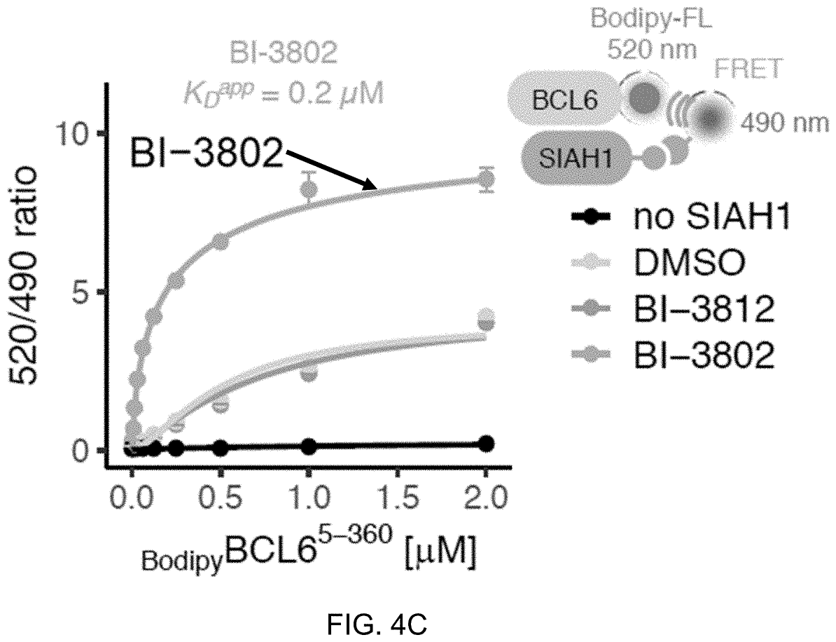

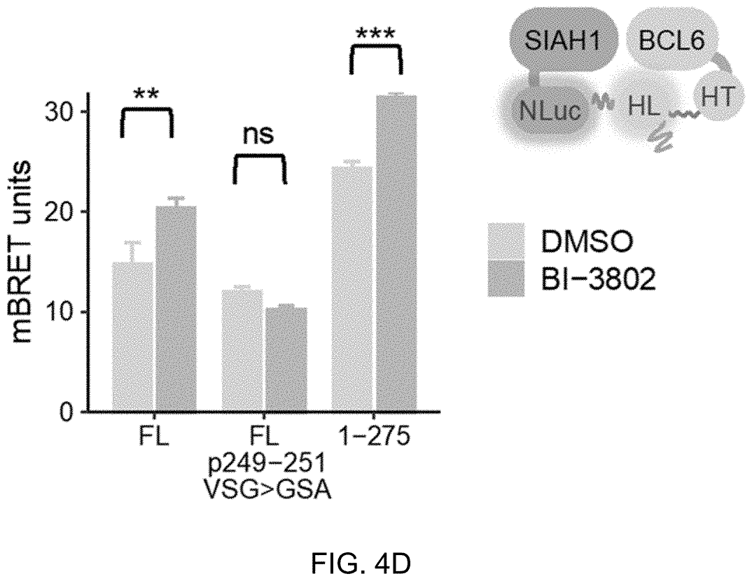

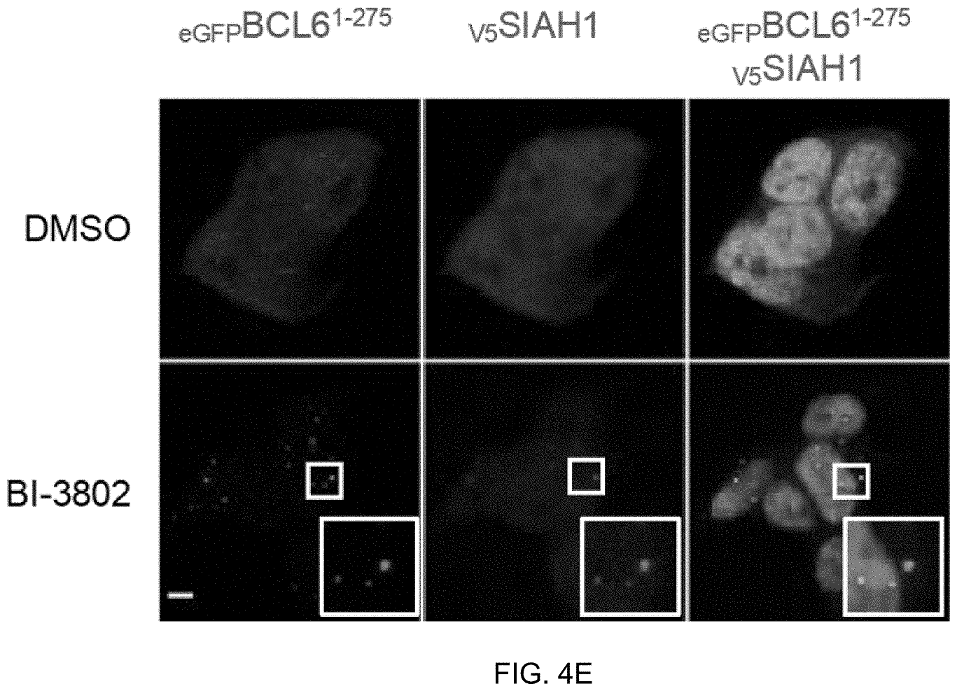

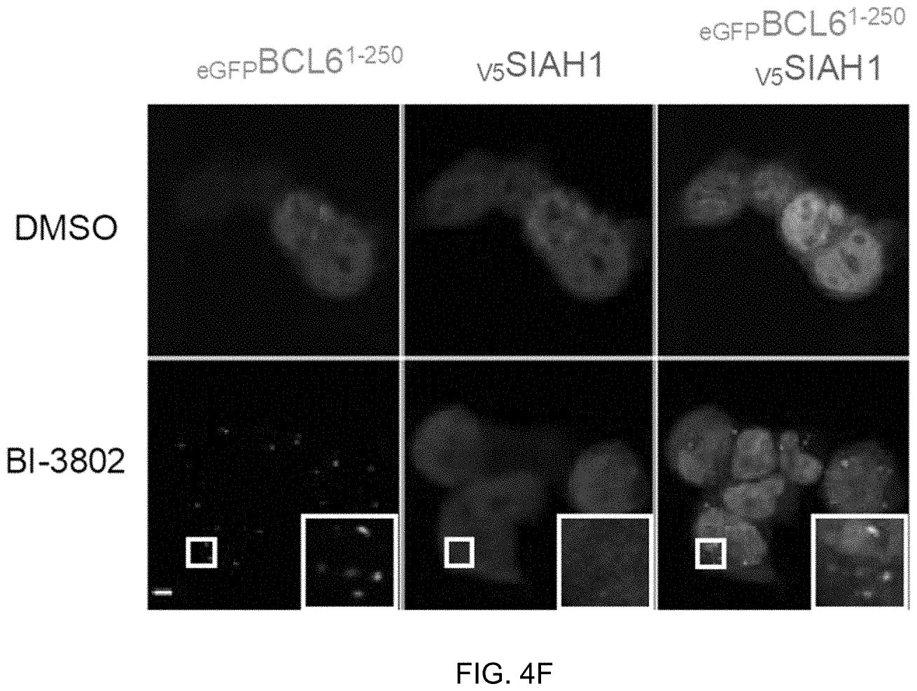

To examine whether the BCL6 V×P motif mediates the interaction with SIAH1, co-immunoprecipitation studies were performed with catalytically inactive SIAH1^{44C>S}. It was found that BCL6 and SIAH1 co-immunoprecipitate in cells (FIG. 4A) and in vitro using recombinant polypeptides (FIG. 10A), and that mutation or deletion of the V×P motif prevented the co-immunoprecipitation. The V×P-containing polypeptide alone (BCL6²⁴¹⁻²⁶⁰) was sufficient for SIAH1 interaction (FIGS. 10B and 10C). In vitro ubiquitination assays with recombinant polypeptides demonstrated that BCL6 is a substrate for SIAH1 (FIG. 10D), and that the rate and magnitude of ubiquitination is accelerated by BI-3802 (FIG. 4B). Together, these data established SIAH1 as a bona-fide E3 ligase for BCL6.

To investigate how BI-3802-induced BCL6 polymerization affects SIAH1-mediated degradation, SIAH1 recruitment and BCL6 ubiquitination were both examined in vitro and in cells. Using a time-resolved fluorescence energy transfer (TR-FRET) assay, moderate baseline affinity was observed between BCL6 and SIAH1, which was strongly enhanced for BI-3802-polymerized BCL6 (K_{D}^{app}=0.2 μM) (FIGS. 4C and 10E). It was found that BI-3802 increased the interaction between BCL6 and SIAH1 (EC₅₀=64 nM) both in vitro (FIG. 10F) and in cells (FIG. 4D), while BI-3812 did not influence the BCL6-SIAH1 interaction, despite comparable affinity of both BI-3802 and BI-3812 to BCL6 (FIG. 10G). Finally, in the presence of BI-3802, SIAH1 colocalized to BCL6 foci in a V×P motif dependent manner (FIGS. 4E and 4F). Together, in vitro and cellular assays indicated that BI-3802-induced polymerization enhanced the interaction between BCL6 and SIAH1, leading to accelerated ubiquitination and degradation of BCL6.

### Example 5: Hybrid Polypeptides

Defined linear degrons were fused to the BCL6-BTB domain (FIGS. 11A and 11C). The ability of BI-3802 to cause degradation of each fusion was evaluated (FIGS. 11B and 11D).

Ten BTB containing polypeptides were chosen and their BTB domain was substituted with the BCL6-BTB domain (FIGS. 12A and 12B). Four fusions did not form foci with and without BI-3802 (BCL6BTB_ZBT12, BCL6BTB_KEAP1, BCL6BTB_KLH22, BCL6BTB_BTBDH). One fusion formed foci without BI-3802 and was not degraded (BCL6BTB_KLHL6). Two fusions formed foci and were not degraded (BCL6BTB_ZBT37, BCL6BTB_KLH35). Three fusions formed foci and were degraded after BI-3802 treatment.

A BISON screen was completed for hybrids. Top hits for BI-3802 E3 ligases+SIAH1 are marked on each plot (FIGS. 13A-13E).

### METHODS OF THE EXAMPLES

The following methods were employed in the above examples.

### Mammalian Cell Culture

The human HEK293T, SuDHL4_{Cas9}, Raji_{Cas9}, and DEL_{Cas9} cell lines were provided by the Genetic Perturbation Platform, Broad Institute. HEK293T_{Cas9} cells were cultured in DMEM (Gibco) and SuDHL4_{Cas9} Raji_{Cas9}, and DEL_{Cas9} cells in RPMI (Gibco), with 10% FBS (Invitrogen), glutamine (Invitrogen) and penicillin-streptomycin (Invitrogen) at 37° C. and 5% CO₂.

### Compounds

BI-3802 and BI-3812 were obtained from opnMe. Boehringer ingelheim, MLN7243 (CT-M7243) from ChemieTek, MLN4924 (HY-70062) from MedChem Express, MG132 (S2619) from Selleck Chemicals, Chloroquine (C6628) from Sigma-Aldrich.

### Antibodies

The following antibodies were used in the above examples: anti-BCL6 (Santa Cruz Biotechnology, sc-7388), anti-beta-tubulin (Cell Signaling, 2146S), anti-Hsp90 (Cell Signaling, 4874S), anti-HDAC1 (Cell Signaling, 2062S), anti-Histone H3 (Cell Signaling, 126485), anti-eGFP (Cell Signaling, 2956), anti-V5-tag (ThermoFisher Scientific, MA5-15253), anti-Streptavidin (Sigma, 71591-3), anti-Mouse 800CW (LI-COR Biosciences, 926-32211), anti-Rabbit 680LT (LI-COR Biosciences, 925-68021), anti-mouse Alexa Fluor 633 (ThermoFisher Scientific, A-21052), and Alexa anti-mouse 488 (Biolegend, 405319).

### Whole Proteome Quantification Using Tandem Mass Tag Mass Spectrometry

10×10⁶ SuDHL4_{Cas9} cells were treated with DMSO, 1 μM BI-3802 or 1 μM BI-3812 for 1 h or 4 h in triplicates and cells were harvested by centrifugation. Samples were processed, measured and analyzed as previously described (Donovan, K. A. et al. Thalidomide promotes degradation of SALL4, a transcription factor implicated in Duane Radial Ray syndrome. Ehfe 7, doi:10.7554/eLife.38430 (2018)). Data are available in the PRIDE repository (PXD016185).

### Cellular Fractionation

1×10⁶ SuDHL4_{Cas9} cells were treated with DMSO or 1 μM BI-3802 for 24 h and fractionated using the CelLytic™NuCLEAR™Extraction Kit (Sigma-Aldrich) according to the manufacturer's protocol, resolved on a polyacrylamide gel, and immunoblotted for the indicated targets.

### Quantitative PCR

1×10⁶ SuDHL4_{Cas9}9 cells were treated with DMSO or 1 μM BI-3802 for 1 h, collected by centrifugation, washed with PBS, and flash frozen in dry ice. mRNA was isolated using the QIAGEN RNA kit (Qiagen, 74106). For cDNA synthesis, total RNA was reverse transcribed with SuperScript™ VILO™ Master Mix (Invitrogen, 11755050) before qPCR analysis with TaqMan Fast Advanced Master Mix (ThermoFisher Scientific, 4444557) for BCL6 (TaqMan, Hs02758991_g1, Life Technologies) and GAPDH (TaqMan, Hs02758991_g1). Reactions were run and analyzed on QStudio 6 FLX real-Time PCR System (ThermoFisher Scientific).

### Immunoblots

SuDHL4_{Cas9}, cells were treated as indicated in figure legends. 2×10⁶ cells were collected (1000 rpm, 5 min) and flash frozen in dry ice. Cells were lysed in 150 μL of lysis buffer (PBS+0.25% NP-40+125 U/ml Benzonase (Invitrogen), 1:100 Halt Protease and Phosphatase Inhibitor Cocktail (Thermo Scientific)) for 2 min at room temperature. The soluble fraction was separated by centrifugation (5000 rpm, 5 min). Protein lysates were mixed with Laemmli (SDS-Sample Buffer, Reducing, 6×, Boston BioProducts), resolved on a polyacrylamide gel, and immunoblotted for the indicated targets.

### Degradation of BCL6 Reporter Constructs in HEK293T Cells

The _{eGFP}BCL6^{FL} BCL6 stability vector was constructed by shuffling BCL6 from pDONR223-BCL6 (Broad Institute human ORFeome library) into a gateway compatible version of “Artichoke” by a LR gateway reaction. _{eGFP}BCL6^{1-250/1-275/1-360/1-500/1-129+Linker+241-260/1-129+Linker+241-260 VSP-GSA} inserts were synthesized or PCR amplified with BsmBI sites and ligated into “Cilantro2” (Addgene #74450) by golden gate assembly. _{eGFP}BCL6^{E41A/G55A/Y58A/C84A/R28A} mutations were designed on the minimal construct containing the BTB domain linked (alternatively, “fused”) to a linker and the SIAH1 binding site (BCL6 1-129+Linker+241-260 VSP>GSA), synthesized through IDT and ligated into “Cilantro2” by golden-gate assembly. Lentivirus was packaged in HEK293T cells using TransIT (Mirus) and subsequently used for spin infection.

HEK293T_{Cas9} cells expressing indicated constructs in “Artichoke” or “Cilantro2” stability reporter vectors (PGK or SFFV Target-eGFP-IRES-mCherry, puromycin resistance) were plated in 96 well plates and treated for indicated times. BCL6-eGFP and mCherry expression were quantified by flow cytometry (CytoFLEX, Beckman or LSR Fortessa flow cytometer BD Biosciences). All degradation assays were done in at least triplicates. Geometric means of eGFP and mCherry fluorescent signals for live and mCherry positive cells were exported using flow cytometry analysis software (FlowJo, BD). Ratios of eGFP to mCherry were normalized to the average of DMSO-treated controls.

### Live Cell Imaging

1×10³ HEK293T cells per cm² were seeded in a μ-Slide 8 Well chamber (ibidi) and cultured for 18-24 h under standard growth conditions. Cell culture medium was exchanged to CO₂ independent media (Gibco) and imaged with the DeltaVision Ultra High-Resolution Microscope (GE Healthcare, 100×lens, oil refraction index=1.520). The following acquisition parameters were used: Image size 896×8% pixels, binning 1×1, GFP exposure time 0.08 sec. and the Neutral Density (% T) filter 32%. To capture foci within all cell volume, around 26μm per cell was imaged every 0.4-0.5 μm. Images were deconvolved (10 cycles, conservative conditions) and projected using maximal intensity by softWoRx® 7.0.0. Images for movies were taken every 10 minutes and combined to a movie by QuickTime.

### Immunofluorescence

HEK293T_{Cas9} cells expressing _{eGFP}BCL6²⁵⁰ and _{eGFP}BCL6²⁷⁵ constructs were transduced with _{V5}SIAH1^{C44S} (infection rate >70%). 0.1×10⁶ cells were plated per chamber of a four-well chamber slide, cultured overnight, pre-treated with 0.5 μM MLN7243 for 2 h, followed by treatment with either DMSO or 2 μM BI-3802 for 1 h. The cells were fixed with 4% formaldehyde for 15 min and permeabilized with 0.1% Triton X100 for 30 min. Epitopes were blocked with 10% BSA for 10 min. Anti-V5 antibodies were added and incubated on slides overnight at 4° C. After removal of the primary antibodies and washes, Alexa Fluor 633-conjugated anti-mouse antibodies were added and incubated at room temperature for 45 min. Finally, the slides were stained with DAPI (BD Biosciences, #564907, 1:5,000 in H2O) and mounted with SlowFader™ Diamond Antifade Mountant (Thermo Fisher Scientific, S36963). Cells were imaged with the Leica TCS SP5 confocal microscope.

### Protein Expression and Purification

The human wild-type and mutant versions of BCL6 (residue 5-129 or 5-360) and SIAH1 variants (residue 90-282 (substrate binding domain, SBD) or full length), were cloned in pAC-derived vectors. Baculovirus for protein expression (Invitrogen) was generated by transfection into Spodoptera frugiperda (Sf9) cells at a density of 0.9×10⁶ cells/mL grown in ESF 921 media (Expression Systems), followed by three rounds of infection in Sf9 cells to increase viral titer. Recombinant proteins were expressed and purified as N-terminal His₆ C-terminal Spy (wild-type and mutant versions of BCL6⁵⁻³⁶⁰), N-terminal Strep II-Avi (BCL6⁵⁻¹²⁹, BCL6⁵⁻³⁶⁰, and SIAH1^{SBD}), and N-terminal Flag (SIAH1^{FL}) fusions in Trichoplusiani High Five insect cells using the baculovirus expression system (Invitrogen).

### Negative Stain Electron Microscopy (EM) Analysis

To prepare grids for negative stain EM analysis of BCL6, Strep II-Avi BCL6⁵⁻³⁶⁰ (0.6 mg/mL, 13.4 μM) in buffer (25 mM HEPES pH 7.4, 200 mM NaCl, 1 mM TCEP) was incubated with DMSO or 20 μM BI-3802 for 1 h at room temperature. The incubated protein samples were rapidly diluted to 10 μg/mL (sample treated with DMSO) or 50 μg/ml (samples treated with BI-3802). A 5 μL aliquot was applied to glow-discharged 400-mesh carbon-coated nickel grids (CF400-NI-UL, Electron Microscopy Sciences). After incubating for 1 min, protein was wicked off with a filter paper and the grid was washed twice with distilled water, followed by two rounds of staining with 2% uranyl acetate for 5 s and 20 s, respectively. Grids were imaged at a nominal magnification of 40,000× on a JEOL JEM-1400Plus operated at 80 kV.

### Docking Simulations and Fiber Visualization

The starting models of the BCL6-BTB domain dimer and BI-3802 were obtained from PDB ID 5MW2. Using RosettaDock4.0, independent local docking simulations (3 Å and 8° moves) were performed by placing two BI-3802-bound BTB domain dimers in three separate starting orientations where BI-3802 was at the interface (viz. end-to-end, end-to-face, and dimers facing each other). No constraints were imposed. The command line used was: $ROSETTA3_BIN/docking_protocol.macosrelease

-nstruct 10000 -partners AB_CD -dock_pert 3 8 -spin
-docking_low_res_score motif_dock_score
-mh:path:scores_BB_BB
$ROSETTA/main/database/additional_protocol_data/motif_dock/xh_16_
-mh:score:use_ss1 false -mh:score:use_ss2 false -mh:score:use_aa1 true
-mh:score:use_aa2 true -ex1 -ex2aro

For each starting orientation, from the 10,000 models generated, 25 top-scoring models (by interface score) were selected. Using PyMOL, more dimers were added to the tetramer model by aligning them in the same binding mode as observed in the model. Many tetramer models could not be extended to produce polymers. For the ones that did, the pitch and the radius of the helix were calculated using HELFIT and compared to those observed in negative stain EM images.

### Construction of the BCL6-BTB Alanine Scan Library

Two 176 bp long oligo libraries were synthesized (Twist Bioscience) in one oligo pool, encoding for BCL6-BTB variants were each amino acid was individually substituted to all four codons of alanine; native alanine codons were substituted to arginine instead. The first library covered BCL6 AA 32-65 (5′-TCCGGAGTCGAGACATCTTGAAATGCAACCTTAGTGTGATCAATC-3′) and the second library covered BCL6 AA 66-99 (5′-CTATAGCATCTTTACAGACCAGTTGGGCAACATCATGGCTGTGAT). The two libraries were amplified from the oligo pool by PCR with the NEBNext polymerase (NEB M0541, 98° C. for 30 sec, 26 cycles of [98° C. for 10 sec, 64° C. for 10 sec. 72° C. for 6 min], 72° C. for 2 min).

The pDONR-BCL6 plasmid backbone was amplified with NEBNext polymerase (98° C. for 30 sec. 6 cycles of [98° C. for 10 sec. 59° C. for 10 sec. 72° C. for 150 sec], 24 cycles of [98° C. for 10 sec, 64° C. for 10 sec, 72° C. for 150 sec], 72° C. for 2 min), dephosphorylated with Dpn1 (NEB), and purified by gel purification using the QIAquick Gel Extraction Kit (Qiagen). Two separate libraries were constructed by Gibson assembly (NEB) (50 ng of the backbone plus 100 ng of the insert, 1 h at 50° C.) and salts were removed by dialysis (membrane filter. 0.025 μm pore size, Millipore). Libraries were transformed into Stbl3 chemical competent bacteria (Invitrogen) and plated on LB plates with carbenicillin for chemical selection. Resulting colonies were scraped, pooled, and purified using the QIAprep Spin Miniprep Kit (Qiagen). To shuffle the Alanine Scan Library into the Artichoke expression backbone, 150 μg of the pDONR BCL6-BTB alanine scan library and 150 μg of the gateway-pArtichoke vector were incubated over night with LR Clonase (ThermoFisher) at room temperature. After Proteinase K treatment, salts were removed by dialysis (membrane filter, 0.025 μm pore size, Millipore). Libraries were transformed into Stbl3 chemical competent bacteria (Invitrogen) and plated on LB plates with carbenicillin for chemical selection. Resulting colonies were scraped, pooled, and purified using the QIAprep Spin Miniprep Kit (Qiagen). Lentivirus for the BCL6-BTB alanine scan library was packaged using HEK293T cells.

### BI-3802 Resistance Screen—Alanine Scan Screen

6×10⁶ SuDhl4_{Cas9} cells were transduced with 5% (v/v) Alanine—Scan 1 or Scan 2 libraries, and selected with 2μg/mL of puromycin 24 h later. 48 h post infections, cells were treated with either DMSO or 1 μM BI-3802. Cells were split every 3-4 days for 21 days and 1×10⁶ cells were harvested for each time point and subjected to direct lysis were flash frozen in dry ice direct lysis buffer adding 1×10⁶ cells/100 μL (1 mM CaCl₂), 3 mM MgCl₂, 1 mM EDTA, 1% Triton X-100, Tris pH 7.5) with freshly supplemented 0.2 mg/mL proteinase. 20 μL of this mix was used for library amplifications in each sorted sample, resulting in 48 first PCR amplification with eight staggered primers in a 50 μL reaction volume (0.04U Titanium Taq (Takara Bio 639210), 0.5× Titanium Taq buffer, 800 μM dNTP mix, 200 nM P5-SBS3 forward primer, 200 nM SBS 12-pXPR003 reverse primer), 94° C. for 5 min, 15 cycles of (94° C. for 30 sec, 58° C. for 15 sec, 72° C. for 30 sec), 72° C. for 2 min. 2 μL of the first PCR reaction was used as the template for 15 cycles of the second PCR, where Illumina adapters and barcodes were added (0.04U Titanium Taq (Takara Bio 639210), 1× Titanium Taq buffer, 800 μM dNTP mix, 200 nM SBS3-Stagger-pXPR003 forward primer, 200 nM P7-barcode-SBS12 reverse primer). An equal amount of all samples was pooled and subjected to preparative agarose electrophoresis followed by gel purification (Qiagen). Eluted DNA was further purified by NaOAc and isopropanol precipitation. Amplified alanine scan libraries were quantified by Illumina's novaseq_sp_100 platform with 123 cycles from SBS3 and 6 barcodes from SBS12. Forward and reverse reads number were combined and analyzed as described below.

### BCL6 Stability—Alanine Scan Screen

6×10⁶ HEK293T_{Cas9} cells were transduced with 5% (v/v) Alanine—Scan 1 or Scan 2 libraries and 24 h later selected with 2 μg/mL of puromycin. Six days post infection cells were treated either with DMSO or 1 μM BI-3802 for 18 h and sorted using FACS. Four populations were collected (top 5%, top 5-15%, low 5-15% and low 5%) based on the eGFP-BCL6/mCherry ratio. For each condition, at least 100×10⁶ cells were subjected for sorting. BCL6 stable (5% highest GFP/mCherry) and BCL6 unstable (5% lowest GFP/mCherry) cells were harvested by centrifugation and cell pellets were flash frozen in dry ice. Sorted cell pellets were resuspended in direct lysis buffer as specified above. Amplified sgRNAs were quantified using Illumina's NextSeq platform.

### Genome-Scale BCL6 Reporter Screen in HEK293T Cells

The puromycin resistance cassette of the _{eGFP}BCL6^{FL} construct was swapped to a neomycin resistance cassette (_{eGFP}BCL6^{FL}-Neo). 5% (v/v) of the human genome-scale CRISPR-KO Brunello library with 0.4 μL polybrene/mL was added to 440×10⁶ HEK293T_{Cas9}) cells expressing _{eGFP}BCL6^{FL}-Neo in 220 mL of RPMI medium. The culture was divided into three replicated and transduced (2400 rpm, 2 h, 37° C.). 24 h post infection sgRNA cells were selected with 2 μg/mL of Puromycin for two days. On the seventh day, cells were treated with either DMSO or 1 μM BI-3802 and then sorted on day eight. Sorted cells were harvested by centrifugation and subjected to direct lysis, library preparation, and sequencing as specified above.

### Targeted BCL6 Reporter Screen in HEK293T Cells

The BISON CRISPR library targets 713 E1, E2, and E3 ubiquitin ligases, deubiquitinases, and control genes and contains 2,852 guide RNAs. The BISON CRISPR library was cloned into the pXPR003 as previously described (Abdulrahman, W. et al. A set of baculovirus transfer vectors for screening of affinity tags and parallel expression strategies. Anal Biochem 385, 383-385, doi:10.1016/j.ab.2008.10.044 (2009)). The virus for the library was produced in a T-175 flask format, as described above with the following adjustments: 1.8×10⁷ HEK293T cells in 25 mL complete DMEM medium, 244 μL of TransIT-LT1, 5 mL of OPTI-MEM, 32 μg of library, 40 μg psPAX2, and 4 μg pVSV-G in 1 mL OPTI-MEM. 10% (v/v) of BISON CRISPR library was added to 6×10⁶ HEK293T_{Cas9} cells in triplicates and transduced. Samples (n=1) were processed as describe above for the genome wide resistance screen.

### Genome-Scale BI-3802 Resistance Screen in SuDHL4 Cells

The resistance screen was performed similarly to the genome-scale BCL6 reporter screen in HEK293T_{Cas9} cells with the following modifications. For three replicates, 500×10⁶ SuDHL4_{Cas9}9 cells in 200 mL of RPMI medium were transduced with 3.5 mL of the human genome-scale CRISPR KO Brunello library with 0.4 μL/mL polybrene. 24 h post infection, cells were selected with 1 μg puromycin/mL for four days. Eight days post infection, cells were exposed to either 1 μM BI-3802 or DMSO. The cells were then cultured for 20 more days until harvesting, with one split every 3-4 days, where fresh drug was added. Genomic DNA was purified with QIAamp DNA Maxi kit (Qiagen) and up to 3 μg of DNA was submitted for multiple reaction 94° C. for 2 min, 18 cycles of (94° C. for 30 sec, 58° C. for 15 sec, 72° C. for 30 sec), 72° C. for 2 min.

### Data Analysis of CRISPR-Cas9 Knockout Screens and Alanine Scans

The data analysis pipeline comprised the following steps: (1) Reads per guide or alanine variant codon for each sample were normalized to the total number of reads across all samples for comparison. (2) For each guide or alanine variant codon, the ratio of reads in the stable vs. unstable sorted gate was calculated, which subsequently was used to rank guide RNAs or alanine variant codons. (3) The replicates were combined by summing up the ranks across replicates for each individual guide or alanine variant codon. (3) The gene or alanine variant rank was then determined as the median rank of the four guides targeting the gene or the four alanine codons encoding the variant. (4) p-values were calculated by simulating a distribution with guide RNAs or alanine variant codons that had randomly assigned ranks over 100 iterations.

### Individual Validation of Alanine Scan Variants

The _{eGFP}BCL6^{FL E41A}, _{eGFP}BCL6^{FL G55A}, _{eGFP}BCL6^{FL Y58A}, and _{eGFP}BCL6^{FL C84A} mutations were introduced by Q5 Site-Directed Mutagenesis (NEB) in pDONR223-BCL6 and then shuffled into the “Artichoke” stability reporter. After the lentivirus production, SuDHL4. Raji and DEL cells were infected with the indicated BCL6 variants and treated with 1 μM BI-3802 or DMSO over 21 days. The percentage of mCherry-positive cells was monitored over time by flow cytometry.

### Single Gene Knockouts

gRNAs targeting genes of interest were cloned into the sgRNA.EFS.tBFP vector using BsmBI digestion. Briefly, vectors were linearized with BsmBI (New England Biolabs) and gel purified (Qiagen spin miniprep). Annealed oligos were phosphorylated with T4 polynucleotide kinase (New England Biolabs), ligated into linearized vector backbone. Constructs were transformed into XL10-Gold ultracompetent Escherichia coli (Stratagene/Agilent Technologies, La Jolla, Calif., USA), plasmids were purified using the MiniPrep Kit (Qiagen) and validated by Sanger sequencing. Lentivirus was produced as described above. HEK293T_{Cas9} or SuDHL4_{Cas9} cells were transduced with sgRNAs. For BCL6 reporter assays, the effect of the knockdown was determined by quantifying the GFP/mCherry ratios in BFP/RFP657 positive and negative populations by flow cytometry seven days post infection. For competition assays, the percentage of BFP positive cells was monitored over time by flow cytometry.

### Overexpression of SIAH1/SIAH2 in HEK293T Cells

HEK293T_{Cas9} cells expressing _{eGFP}BCL6^{FL} were transduced with _{V5}SIAH1, _{V5}SIAH1^{44C>S}, or _{V5}SIAH2. Cells were trypsinized 72 h after infection and eGFP and mCherry expression quantified by flow cytometry. For construction of _{V5}SIAH1. _{V5}SIAH2 expression vectors, inserts were PCR amplified with attP sites and cloned into pDONR221 by a BP clonase reaction and then transferred into the pLEX_307 (Addgene #41392) expression vector by a LR clonase reaction. To construct _{V5}SIAH1^{44C>S}, mutations were introduced by site-directed mutagenesis in pDONR221-SIAH1 and then transferred into pLEX_307 (Addgene #41392).

### Co-Immunoprecipitation

HEK293T_{Cas9} cells expressing _{eGFP}BCL6^{FL}, _{eGFP}BCL6^{FL 249-251 VSP>GSA}, _{eGFP}BCL6¹⁻²⁵⁰, and _{eGFP}BCL6¹⁻²⁷⁵ constructs were transduced with _{V5}SIAH1^{C44S}. 1×10⁶ cells were plated into 10 cm dishes, cultured for one day, treated with 0.5 μM MLN7249 for 2 h, and then with either 2 μM BI-3802 or DMSO for 1 h. The cells were harvested and lysed in RIPA lysis buffer (ThermoFisher Scientific, #89900) infused with protease inhibitor (ThermoFisher Scientific, Halt™ Protease Inhibitor Cocktail #78438) for 30 min at 4° C. 5 μM BI-3802 was infused to all buffers used for the BI-3802 treated arm. Lysates were cleared by centrifugation (17,000 g, 20 min, 4° C.). 20 μL of pre-cleaned GFP-trap magnetic agarose beads (Chromotek, gmta-20) was added to the lysates. The beads-lysate mix was incubated at 4° C. for 30 min. Proteins were eluted in 2×sample buffer at 98° C. Eluates and whole-cell lysates were run on a polyacrylamide gel, transferred to a nitrocellulose membrane and immunoblotted for eGFP and V5.

### In Vitro Pull Down

For the pull-downs of BCL6 (Strep II-Avi BCL6⁵⁻¹²⁹ or BCL6⁵⁻³⁶⁰) with SIAH1 (tag-cleaved SIAH1^{SBD}), 20 μM BCL6 variants and 30 μM SIAH1^{SBD} were incubated in 300 μL binding buffer (25 mM HEPES pH 7.4, 200 mM NaCl, 2 mM TCEP) with 2 μM BI-3802 (0.5% DMSO) for 1 h. 50 μL of Strep-Tactin XT Superflow (IBA) beads were added and incubated for another 1 h. Beads were washed quickly three times with 100 μL of washing buffer, and samples were eluted with 100 μL of elution buffer (binding buffer with 50 mM Biotin). All samples were analyzed by SDS-PAGE.

### Isothermal Titration Calorimetry (ITC)

All calorimetric experiments were carried out using an Affinity ITC from TA Instruments (New Castle, Del.) equipped with auto sampler in a buffer containing 20 mM HEPES, pH 7.5, 150 mM NaCl, and 0.5 mM TCEP at 25° C. For the BCL6-SIAH1 interaction, 25 μM BCL6⁵⁻³⁶⁰ protein solution in the calorimetric cell was titrated by injecting 2 μL of 250 μM SIAH1^{SBD} protein solution in 200 sec intervals with stirring speed at 125 rpm. For the isolated BCL6 peptide (residues 241-260) and SIAH1 interaction, 25 μM SIAH1^{SBD} protein solution in the calorimetric cell was titrated by injecting 2 μL of 250 μM BCL6²⁶¹⁻²⁶⁰ polypeptide solution in a same setup. Resulting isotherm was fitted with a single site model to yield thermodynamic parameters of DH, DS, stoichiometry, and K_{d} using NanoAnalyze software (TA instruments).

### BCL6-SIAH1 Time-Resolved Fluorescence Resonance Energy Transfer (TR-FRET)

Titrations of compounds to induce BCL6⁵⁻²⁶⁰-SIAH1 complex were carried out by mixing 200 nM biotinylated Strep II Avi-tagged SIAH1^{SBD}, 200 nM BodipyFL-labeled BCL6⁵⁻³⁶⁰ variants, and 2 nM terbium-coupled streptavidin (Invitrogen) in an assay buffer containing 50 mM Tris pH 8.0, 200 mM NaCl, 0.1% Pluronic F-68 solution (Sigma), 0.5% BSA (w/v), 1 mM TCEP. After dispensing the assay mixture (15 μL volume), increasing concentrations of compounds were dispensed in a 384-well microplate (Corning, 4514) using a D300e Digital Dispenser (HP) normalized to 1% DMSO. After excitation of terbium fluorescence at 337 nm, emission at 490 nm (terbium) and 520 nm (BodipyFL) were recorded with a 70 us delay over 600 μs to reduce background fluorescence, and the reaction was followed over 60 cycles of each data point using a PHERAstar FS microplate reader (BMG Labtech). The TR-FRET signal of each data point was extracted by calculating the 520/490 nm ratio. The half-maximal effective concentration (EC₅₀) values were estimated using dose-response analysis standard four parameter log-logistic curves, fitted to the experimental data using the dr4pl R package.

Titrations of BodipyFL-BCL6⁵⁻³⁶⁰ were carried out by mixing 400 nM biotinylated Strep II Avi-tagged SIAH1^{SBD}, 2 μM compounds or equivalent volume of DMSO, and 4 nM terbium-coupled streptavidin in the same assay buffer. After dispensing the assay mixture, increasing concentration of BodipyFL-BCL6⁵⁻³⁶⁰ was added to the SIAH1 mixture in a 1.1 volume ratio (7.5 μL each, total 15 μL assay volume). The 520/490 nm ratios were measured in as described above and plotted to calculate the K_{D}^{APP} values using dose-response analysis standard four parameter log-logistic curves using the dr4pl R package.

### BCL6-BCoR TR-FRET (Compound Binding Assay)

Competitive titration of BI-3802 or BI-3812 were carried out by mixing 100 nM biotinylated BCL6⁵⁻¹²⁹, 100 nM N-terminal FITC-labeled BCoR peptide (sequence: RSEIISTAPSSWVVPGP), and 2 nM terbium-coupled streptavidin in the same assay buffer. After dispensing the assay mixture (15 μL volume), increasing concentrations of compounds were dispensed in a 384-well microplate (Corning, 4514) using a D300e Digital Dispenser (HP) normalized to 1% DMSO. The 520/490 nm ratios were measured as described above and plotted to calculate the K_{D}^{APP} values using dose-response analysis standard four parameter log-logistic curves using the dr4pl R package.

### BRET Analysis

Bioluminescence resonance energy transfer (BRET) experiments were performed using a NanoBRET PPI starter kit (Promega N1821) according to the manufacturer's instructions and as previously described (Sperling, A. S. et al. Patterns of substrate affinity, competition, and degradation kinetics underlie biological activity of thalidomide analogs. Blood 134, 160-170, doi:10.1182/blood.2019000789 (2019)).

### In Vitro Ubiquitination

In vitro ubiquitination for identification of compatible E2 conjugating enzymes was performed by following the manufacturer's instructions (K-982, Boston Biochem), using Strep II-Avi-BCL6⁵⁻³⁶⁰ and Flag-SIAH1^{FL}. Time-course in vitro ubiquitination was performed by mixing the substrate (BCL6, 2 μM), E3 (SIAH, 0.5 μM), E1 (UBA1, Boston Biochem, 0.2 μM, E2 (UBE2D1, Boston Biochem, 0.5 μM), and ubiquitin (Boston Biochem, 50 μM), with a reaction buffer (B-71, Boston Biochem) containing BI-3802 or DMSO (normalized to 1% DMSO) in 15 μL volume each. Reactions were initiated by adding 5 μL of Mg-ATP solution (B-20, Boston Biochem), incubated for up to 60 min at 37° C., and analyzed by western blot using Strep tag II Antibody HRP conjugate (71591-3, Sigma) at 1:4,000. 0.8×10⁶ cells/mL SuDHL4 cells were treated with DMSO or 0.5 μM E1 Inhibitor (3 h)+1 μM BI-3802 (I h) and 200 μL of the cell suspension was immobilized on a slide using the Cytospin™ 4 Cytocentrifuge (CytoSpin 4, A78300003; 6000 rpm, 6 min). Medium was aspirated and cells were fixed with 4% formaldehyde diluted in warm PBS for 15 min at room temperature. Slides were washed three times for 5 min with PBS, blocked and permeabilized with blocking solution (5% Normal Goat Serum (Cell Signaling), 0.3% Triton X-100 in PBS) for 60 min and stained with anti-BCL6 antibody in blocking solution overnight at 4° C. Cells were washed three times with PBS for 5 min each, incubated with Alexa Fluor 488-conjugated anti-mouse antibodies, washed three times with PBS for 5 min each and covered with coverslip slides using Prolong®Gold Antifade Reagent (ThermoFisher, P36934). Cells were imaged using the DeltaVision microscope as described above.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A recombinant polypeptide comprising a fragment of a BCL6 polypeptide, wherein the fragment comprises a Broad complex/Tamtrack/Brick-a-brack (BTB) domain of the BCL6 polypeptide.

2. A fusion polypeptide comprising the BTB domain of claim 1 linked to a heterologous amino acid sequence.

3. The polypeptide of claim 1, wherein the polypeptide further comprises a degron.

4. The polypeptide of claim 1, wherein the polypeptide comprises an amino acid linker at an N-terminus or a C-terminus of the BTB domain.

5. The polypeptide of claim 3, wherein the degron comprises at least about 20 amino acid residues of a BCL6 polypeptide.

6. The polypeptide of claim 3, wherein the degron comprises a V×P binding motif.

7. The polypeptide of claim 1, wherein the polypeptide comprises an alteration at amino acid R28, E41, C84, G55, or Y58.

8. The polypeptide of claim 1, wherein the polypeptide is polymerizable.

9. The polypeptide of claim 8, wherein polymerization of the polypeptide enhances degradation of the polypeptide.

10. The polypeptide of claim 8, wherein polymerization of the polypeptide is induced by a compound binding to the BTB domain.

11. The polypeptide of claim 10 wherein the BTB comprises a hydrophobic residue that mediates polymerization of the polypeptide upon binding of the compound.

12. The polypeptide of claim 10, wherein the compound is a quinolinone or a benzimidazolone.

13. The polypeptide of claim 10, wherein the compound is selected from the group consisting of BI-3802 (2-((6-((5-chloro-2-((3S,5R)-3,5-dimethylpiperidin-1-yl)pyrimidin-4-yl)amino)-1-methyl-2-oxo-1,2-dihydroquinolin-3-yl)oxy)-N-methylacetamide); 5-((5-Chloro-2-(3-methylpiperidin-1-yl)pyrimidin-4-yl)-amino)-3-(3-hydroxy-3-methylbutyl)-1-methyl-1,3-dihydro-2H-benzo[d]imidazol-2-one; 5-((5-Chloro-2-((3S,5R)-3,5-dimethylpiperidin-1-yl)-pyrimidin-4-yl)amino)-3-(3-hydroxy-3-methylbutyl)-1-methyl-1,3-dihydro-2H-benzo[d]imidazol-2-one; 5-((5-Chloro-2-((3S,5R)-3,5-dimethylpiperidin-1-yl)pyridin-4-yl)amino)-3-(3-hydroxy-3-methylbutyl)-1-methyl-1,3-dihydro-2H-benzo[d]imidazol-2-one; 5-((5-Chloro-2-(3-(trifluoromethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(4,4-difluoropiperidin-1-yl)pyrimidin-4-yl)-amino)-3-(3-hydroxy-3-methylbutyl)-1-methyl-1,3-dihydro-2H-benzo[d]imidazol-2-one; 5-((5-Chloro-2-(4,4-difluoro-3-methylpiperidin-1-yl)-pyrimidin-4-yl)amino)-3-(3-hydroxy-3-methylbutyl)-1-methyl-1,3-dihydro-2H-benzo[d]imidazol-2-one; 5-((5-Chloro-2-((3R,5S)-4,4-difluoro-3,5-dimethylpiperidin-1-yl)pyrimidin-4-yl)amino)-3-(3-hydroxy-3-methylbutyl)-1-methyl-1,3-dihydro-2H-benzo-[d]imidazol-2-one, 5-((5-Chloro-2-(3-(hydroxymethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(4,4-difluoro-3-(hydroxymethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(4,4-difluoro-3-(methoxymethyl)piperidin-1-yl)pyrimidin-4-yl)amino)-3-(3-hydroxy-3-methylbutyl)-1-methyl-1,3-dihydro-2H-benzo[d]imidazol-2-one; 5-((5-Chloro-2-(dimethylamino)pyrimidin-4-yl)amino)-3-(3-hydroxy-3-methylbutyl)-1-methyl-1,3-dihydro-2H-benzo[d]-imidazol-2-one; 5-((5-Chloro-2-morpholinopyrimidin-4-yl)amino)-3-(3-hydroxy-3-methylbutyl)-1-methyl-1,3-dihydro-2H-benzo[d]-imidazol-2-one; 5-((5-Chloro-2-(piperidin-1-yl)pyrimidin-4-yl)amino)-3-(3-hydroxy-3-methylbutyl)-1-methyl-1,3-dihydro-2H-benzo[d]-imidazol-2-one; 5-((5-Chloro-2-((2R,6S)-2,6-dimethylmorpholino)pyridin-4-yl)amino)-3-(3-hydroxy-3-methylbutyl)-1-methyl-1,3-dihydro-2H-benzo[d]imidazol-2-one; 2-Chloro-4-((3-(3-hydroxy-3-methylbutyl)-1-methyl-2-oxo-2,3-dihydro-1H-benzo[d]imidazol-5-yl)amino)nicotinonitrile; 5-((2,3-Dichloropyridin-4-yl)amino)-3-(3-hydroxy-3-methylbutyl)-1-methyl-1,3-dihydro-2H-benzo[d]imidazol-2-one; 5-((3-Chloropyridin-4-yl)amino)-3-(3-hydroxy-3-methylbutyl)-1-methyl-1,3-dihydro-2H-benzo[d]imidazol-2-one; 4-Chloro-6-((3-(3-hydroxy-3-methylbutyl)-1-methyl-2-oxo-2,3-dihydro-1H-benzo[d]imidazol-5-yl)amino)pyrimidine-5-Carbonitrile; 5-((5,6-Dichloropyrimidin-4-yl)amino)-3-(3-hydroxy-3-methylbutyl)-1-methyl-1,3-dihydro-2H-benzo[d]imidazol-2-one; 5-((3,5-Dichloropyridin-4-yl)amino)-3-(3-hydroxy-3-methylbutyl)-1-methyl-1,3-dihydro-2H-benzo[d]imidazol-2-one; 5-((5-Chloro-2-(methylthio)pyrimidin-4-yl)amino)-3-(3-hydroxy-3-methylbutyl)-1-methyl-1,3-dihydro-2H-benzo[d]-imidazol-2-one; 5-((2,5-Dichloropyrimidin-4-yl)amino)-3-(3-hydroxy-3-methylbutyl)-1-methyl-1,3-dihydro-2H-benzo[d]imidazol-2-one; 2-Chloro-4-((cyclopropylmethyl)amino)nicotinonitrile; 3,4,2-Chloro-4-((1,3-dimethyl-2-oxo-2,3-dihydro-1H-benzo[d]-imidazol-5-yl)amino)nicotinonitrile; 2-Chloro-4-((3-(2-hydroxybutyl)-1-methyl-2-oxo-2,3-dihydro-1H-benzo[d]imidazol-5-yl)amino)nicotinonitrile; 2-Chloro-4-((3-(2-cyanobutyl)-1-methyl-2-oxo-2,3-dihydro-1H-benzo[d]imidazol-5-yl)amino)nicotinonitrile; (S)-2-Chloro-4-((3-(2-hydroxybutyl)-1-methyl-2-oxo-2,3-dihydro-1H-benzo[d]imidazol-5-yl)amino)nicotinonitrile; (R)-2-Chloro-4-((3-(2-hydroxybutyl)-1-methyl-2-oxo-2,3-dihydro-1H-benzo[d]imidazol-5-yl)amino)nicotinonitrile; 4-((3-Butyl-1-methyl-2-oxo-2,3-dihydro-1H-benzo[d]-imidazol-5-yl)amino)-2-chloronicotinonitrile; (R)-2-Chloro-4-((3-(3-hydroxybutyl)-1-methyl-2-oxo-2,3-dihydro-1H-benzo[d]imidazol-5-yl)amino)nicotinonitrile; (S)-2-Chloro-4-((3-(3-hydroxybutyl)-1-methyl-2-oxo-2,3-dihydro-1H-benzo[d]imidazol-5-yl)amino)nicotinonitrile; 1-Methyl-5-nitro-1,3-dihydro-2H-benzo[d]imidazol-2-one; 2-Chloro-4-((1-methyl-2-oxo-2,3-dihydro-1H-benzo[d]-imidazol-5-yl)amino)nicotinonitrile; 3-(2-Hydroxybutyl)-1-methyl-5-nitro-1,3-dihydro-2Hbenzo[d]imidazol-2-one; 3-Hydroxy-3-methylbutyl 4-methylbenzenesulfonate; [(3R)-3-Hydroxybutyl] 4-methylbenzenesulfonate; 3-(3-Hydroxy-3-methylbutyl)-1-methyl-5-nitro-1,3-dihydro-2H-benzo[d]imidazol-2-one; 5-Amino-3-(3-hydroxy-3-methylbutyl)-1-methyl-1,3-dihydro-2H-benzo[d]imidazol-2-one; 5-((2-Bromo-5-chloropyridin-4-yl)amino)-3-(3-hydroxy-3-methylbutyl)-1-methyl-1,3-dihydro-2H-benzo[d]imidazol-2-one; 5-Chloro-2-((3S,5R)-3,5-dimethylpiperidin-1-yl)-4-iodopyridine; (2S,6R)-4-(5-Chloro-4-iodopyridin-2-yl)-2,6-dimethylmorpholine; and various combinations thereof.

14. A nucleotide molecule encoding the recombinant polypeptide of claim 1.

15. An expression vector comprising the nucleotide molecule of claim 14.

16. A cell comprising the nucleotide molecule of claim 14.

17. A method for inducing polymerization of a polypeptide of interest, the method comprising operably linking the polypeptide of interest to a BTB domain of claim 1 to generate a fusion polypeptide, and contacting the fusion polypeptide with a compound that induces polymerization of the BTB domain, thereby inducing polymerization of the fusion polypeptide.

18. The method of claim 17, wherein polymerization of the fusion polypeptide enhances degradation of the fusion polypeptide.

19. A method for reversing polymerization of a polypeptide, the method comprising contacting a fusion polypeptide with a BCL6 inhibitor after the fusion polypeptide has been polymerized by the method of claim 17, wherein contacting the fusion polypeptide with the BCL6 inhibitor causes the BTB domain to depolymerize, thereby reversing polymerization of the polypeptide.

20. A method for measuring efficacy of an agent in inducing degradation of a fusion polypeptide comprising a BTB domain and expressed by a cell, the method comprising:
(a) contacting a cell expressing a detectable fusion polypeptide comprising the BTB domain with an agent to be evaluated for inducing degradation of the fusion polypeptide; and
(b) detecting the level of fusion polypeptide present in the cell after being contacted with the agent relative to the level of fusion polypeptide present in a corresponding control cell, wherein a reduction in the level of fusion polypeptide identifies the agent as effective in inducing degradation of the polypeptide comprising the BTB domain.

21. A method for selecting a subject for treatment with a compound that induces degradation of a BCL6 polypeptide, the method comprising detecting in a biological sample of the subject the presence or absence of an alteration at amino acid R28, E41, C84, G55, Y58 or BCL6, wherein the absence of the alteration selects the subject for treatment with a compound that induces degradation of a BCL6 polypeptide.

22. A biomaterial prepared by a method comprising operably linking a polypeptide of interest to the BTB domain of claim 1 to generate a fusion polypeptide, and contacting the fusion polypeptide with a compound that induces polymerization of the BTB domain, thereby inducing polymerization of the fusion polypeptide and formation of the biomaterial.
